(19) United States
(12) Patent Application Publication
(10) Pub. No.: US 2021/0270817 A1
(43) Pub. Date: Sep. 2, 2021

(54) Methods for Assaying Binding Affinity

(71) Applicant: Berkeley Lights, Inc., Emeryville, CA (US)

(72) Inventors: Paul M. Lebel, Redwood City, CA (US); Troy A. Lionberger, San Francisco, CA (US); Kevin T. Chapman, Emeryville, CA (US)

(73) Assignee: Berkeley Lights, Inc., Emeryville, CA (US)

(21) Appl. No.: 17/198,833

(22) Filed: Mar. 11, 2021

Related U.S. Application Data
(63) Continuation of application No. PCT/US2019/051129, filed on Sep. 13, 2019.
(60) Provisional application No. 62/731,123, filed on Sep. 14, 2018.

(51) Int. Cl. G01N 33/53; B01L 3/00

(52) U.S. Cl. CPC G01N 33/53; B01L 3/502707; B01L 3/502761; B01L 2400/0427; B01L 2300/161; B01L 2400/0424; B01L 2300/0645

(57) ABSTRACT

Disclosed herein is a method for assaying binding affinity between a first molecule and a second molecule in a micro-fluidic device.

This application is a continuation of International Patent Application No. PCT/US2019/051129, filed Sep. 13, 2019, which claims the benefit of priority of U.S. Provisional Patent Application No. 62/731,123, filed Sep. 14, 2018, the contents of each of which are incorporated herein by reference for its entirety.

### INTRODUCTION AND SUMMARY

Scientists have long been interested in measuring the binding affinity between molecules that specifically interact with one another. For protein therapeutics, surface plasmon resonance (SPR) has become the most widely accepted technique for determining binding affinities. However, SPR requires costly equipment that is exclusively dedicated to the measurement of binding affinity and requires a large amount of highly purified material. Given these drawbacks to SPR, which limits its use to only a limited number of candidate molecules, there is a need for new approaches to the measurement of binding affinity that require less preparatory work and can be performed at larger scale.

The present disclosure provides methods for assaying a binding affinity between a first molecule and a second molecule. The micro-fluidic device comprises a flow region and a chamber that opens off of the flow region.

In some embodiments, the methods comprise: providing the second molecule into the chamber, wherein the second molecule is labeled with a signal-emitting moiety and a first capture micro-object comprising the first molecule is present in the chamber; removing unbound second molecule from the microfluidic device; providing a second capture micro-object into the chamber, wherein the second capture micro-object comprises a third molecule which specifically binds to the second molecule; detecting over a period of time a decrease in an amount of second molecule bound to the first capture micro-object; and determining a relative binding affinity between the first molecule and the second molecule.

In some embodiments, providing the second molecule into the chamber further comprises allowing the second molecule to bind to the first molecule of the first capture micro-object. In some embodiments, the binding of the second molecule to the first molecule is allowed to proceed to saturation. In some embodiments, the methods further comprise detecting over the period of time an increase in the amount of second molecule bound to the second capture micro-object.

In some embodiments, the binding affinity between the first molecule and the second molecule is determined based on one of the following: the decrease in the amount of second molecule bound to the first capture micro-object over the period of time, or a ratio of (i) the increase in the amount of second molecule bound to the second capture micro-object over the period of time to (ii) the decrease in the amount of second molecule bound to the first capture micro-object over the period of time.

In certain embodiments, the methods comprise: providing a second molecule labeled with a signal-emitting moiety into the chamber, wherein a first capture micro-object comprising the first molecule is present in the chamber; removing unbound second molecule from the microfluidic device; detecting over a period of time a decrease in the amount of the second molecule bound to the first capture micro-object; and determining a relative binding affinity between the first molecule and the second molecule.

In some embodiments, providing a second molecule labeled with a signal-emitting moiety into the chamber further comprises allowing the second molecule to bind to the first molecule of the first capture micro-object. In some embodiments, the binding of the second molecule to the first molecule is allowed to proceed to saturation. In some embodiments, the binding affinity between the first molecule and the second molecule is determined based on the decrease in amount of second molecule bound to the first capture micro-object over the period of time.

In certain embodiments, methods for assaying binding affinities of a target molecule and each of a plurality of distinct binding partners in a micro-fluidic device are provided. The micro-fluidic device comprises a flow region and a plurality of chambers that open off of the flow region. In certain embodiments, the methods comprise: providing the target molecule into the plurality of chambers, wherein the target molecule is labeled with a signal-emitting moiety and wherein a first plurality of capture micro-objects, each comprising a distinct binding partner, are present in the plurality of chambers; removing unbound target molecule from the microfluidic device; providing a second plurality of capture micro-objects into the plurality of chambers, wherein each of the capture micro-objects of the second plurality comprises a binding partner for the target molecule; detecting over a period of time a decrease in the amount of target molecule bound to the capture micro-objects of the first plurality; determining relative binding affinities of the target molecule and each of the plurality of distinct binding partners.

In some embodiments, providing the target molecule into the plurality of chambers further comprises allowing the target molecule to bind to the binding partners of the capture micro-objects of the first plurality. In some embodiments, the binding of the target molecule to the binding partners is allowed to proceed to saturation. In some embodiments, the methods further comprise detecting over the period of time an increase in the amount of target molecule bound to the capture micro-objects of the second plurality.

In some embodiments, the relative binding affinities between the target molecule and each of the plurality of distinct binding partners are determined based on (1) decreases in the amount of target molecule bound to the capture micro-objects of the first plurality over the period of time, or (2) ratios of (i) increases in the amount of target molecule bound to the capture micro-objects of the second plurality over the period of time to (ii) decreases in the amount of target molecule bound to the capture micro-objects of the first plurality over the period of time.

In certain embodiments, methods for assaying binding affinities of a target molecule and one or more binding partners for the target molecule in a micro-fluidic device are provided. The micro-fluidic device comprises a flow region and a chamber that opens off of the flow region. In certain embodiments, the methods comprise: providing the target molecule into the chamber, wherein the target molecule is labeled with a signal-emitting moiety and wherein a first capture micro-object comprising a first binding partner is present in the chamber; removing unbound target molecule from the microfluidic device; providing a second capture micro-object into the chamber, wherein the second capture micro-object comprises a second binding partner different from the first binding partner; detecting over a period of time a decrease in the amount of target molecule bound to the first capture micro-object; determining a relative binding affinity of the target molecule and the first binding partner.

In some embodiments, providing the target molecule into the chamber further comprises allowing the target molecule to bind to the first binding partner of the first capture micro-object, wherein the binding of the target molecule to the first binding partner is allowed to proceed to saturation. In some embodiments, the methods further comprise detecting over the period of time an increase in the amount of target molecule bound to the second capture micro-object.

In some embodiments, the relative binding affinity of the target molecule and the first binding partner is determined based on (1) the decrease in the amount of target molecule bound to the first capture micro-object over the period of time, or (2) a ratio of (i) the increase in the amount of target molecule bound to the second capture micro-object over the period of time to (ii) the decrease in the amount of target molecule bound to the first capture micro-object over the period of time.

These and other features and advantages of the disclosed methods will be set forth or will become more fully apparent in the description that follows and in the appended claims. The features and advantages may be realized and obtained by means of the objects and combinations particularly pointed out in the appended examples, partial listing of embodiments, and claims. Furthermore, the features and advantages of the described methods may be learned by the practice or will be obvious from the description, as set forth hereinafter.

### BRIEF DESCRIPTION OF THE DRAWINGS

FIG. 1A illustrates an example of a system for use with a microfluidic device and associated control equipment according to some embodiments of the disclosure.

FIGS. 1B and 1C illustrate a microfluidic device according to some embodiments of the disclosure.

FIGS. 2A and 2B illustrate isolation pens according to some embodiments of the disclosure.

FIG. 2C illustrates a detailed sequestration pen according to some embodiments of the disclosure.

FIGS. 2D-2G illustrate sequestration pens according to some other embodiments of the disclosure.

FIG. 2H illustrates a microfluidic device according to an embodiment of the disclosure.

FIG. 3 illustrates a coated surface of the microfluidic device according to an embodiment of the disclosure.

FIG. 4A illustrates a specific example of a system for use with a microfluidic device and associated control equipment according to some embodiments of the disclosure.

FIG. 4B illustrates an imaging device according to some embodiments of the disclosure.

FIGS. 5A-5B illustrate exemplary structural parameters of an exemplary microfluidic device as well as adjustable parameters that can be configured for the assay, according to some embodiments of the disclosure, according to some embodiments of the disclosure. FIG. 5A shows an embodiment with a single first capture micro-object and a single second capture micro-object; FIG. 5B shows an embodiment having a single first capture micro-object and a plurality of second capture micro-objects.

FIGS. 5C-5D illustrate exemplary structural parameters of an exemplary microfluidic device as well as adjustable parameters that can be configured for the assay, according to some embodiments of the disclosure.

FIG. 6A illustrates a kinetic rate model illustrating transition rates in the source-capture assay. One capture bead and one source bead are shown, but the model can be generalized to multiple capture beads and one source, multiple source and one capture, or multiple capture and multiple source beads.

FIG. 6B illustrates an expected change in fluorescence intensity of first and second capture micro-objects according to some embodiments of the disclosure.

FIG. 7A illustrates steps of a method for performing a binding affinity assay between a first micro-object and a second micro-object, according to some embodiments of the disclosure.

FIG. 7B illustrates a method for performing the assay on an array of molecules with differing binding affinities, according to some embodiments of the disclosure.

FIG. 8A illustrates the use of first and second capture micro-objects to assay binding affinity between a first molecule and a second molecule according to some embodiments of the disclosure.

FIGS. 8B-8C provide images of a microfluidic device in which first and second capture micro-objects are being used to assay binding affinity between a first molecule and a second molecule according to some embodiments of the disclosure.

FIG. 8D provides graphical displays of the change in the ratio of fluorescent intensity of the second capture micro-object to the first capture micro-object over time according to some embodiments of the disclosure.

FIGS. 8E-8G show relative assessments of binding affinity which relies upon the use of first and second capture micro-objects to assay binding affinity between a first molecule and a second molecule according to some embodiments of the disclosure.

FIGS. 9A-9C illustrate a method for performing the assay on an array of molecules with differing binding affinities, according to some embodiments of the disclosure.

### DETAILED DESCRIPTION

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on”, “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

### I. Definitions

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. In some embodiments, when used with respect to numerical values or parameters or characteristics that can be expressed as numerical values, “substantially” means within ten percent.

Numeric ranges are inclusive of the numbers defining the range.

“Or” is used in the inclusive sense, i.e., equivalent to “and/or,” unless the context requires otherwise.

The term “ones” means more than one.

As used herein, the term “plurality” can be 2, 3, 4, 5, 6, 7, 8, 9, 10, or more.

As used herein: μm means micrometer, μm³ means cubic micrometer, pL means picoliter, nL means nanoliter, and μL (or uL) means microliter.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “obstruction” refers generally to a bump or similar type of structure that is sufficiently large so as to partially (but not completely) impede movement of target micro-objects between two different regions or circuit elements in a microfluidic device. The two different regions/circuit elements can be, for example, the connection region and the isolation region of a microfluidic sequestration pen.

As used herein, the term “constriction” refers generally to a narrowing of a width of a circuit element (or an interface between two circuit elements) in a microfluidic device. The constriction can be located, for example, at the interface between the isolation region and the connection region of a microfluidic sequestration pen of the instant disclosure.

As used herein, the term “transparent” refers to a material which allows visible light to pass through without substantially altering the light as is passes through.

As used herein, the term “saturation” refers to the state where target molecules bind to substantially all of the target-specific binding partners available on a capture micro-object(s) in a same chamber or sequestration pen.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts (as described, for example, in Ritchie et al. (2009) “Reconstitution of Membrane Proteins in Phospholipid Bilayer Nanodiscs,” Methods Enzymol., 464:211-231), and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, nucleic acids (e.g., oligonucleotides), proteins, carbohydrates, antigens, small molecule signaling moieties, or other chemical/biological species capable of use in an assay.

As used herein, a “distance” between the micro-objects is measured between the center of the micro-objects.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein, “capture moiety” is a chemical or biological species, functionality, or motif that provides a recognition site for a micro-object. A selected class of micro-objects may recognize the in situ-generated capture moiety and may bind or have an affinity for the in situ-generated capture moiety. Non-limiting examples include antigens, antibodies, and cell surface binding motifs.

As used herein, the term “signal-emitting moiety” (also known as a label) assists the user by enabling detection of a molecule (e.g., target or target-specific binding partner) to which it binds directly or indirectly. When the disclosure refers to detecting a molecule (or an amount or change in amount thereof), the disclosure references detecting the signal produced by the signal-emitting moiety bound to the molecule. Various optical or non-optical signal-emitting moieties may be employed for signaling purposes. In some embodiments, the signal-emitting moiety is optically observable. In some embodiments, the signal-emitting moiety is a signal emitting molecule that fluoresce may be used, such as organic small molecules, including, but not limited to fluorophores, such as, but not limited to, fluorescein, Texas Red, Rhodamine, cyanine dyes, Alexa dyes, DyLight dyes, Atto dyes, etc. In some embodiments, organic polymers, such as p-dots may be employed. In some embodiments, the signal-emitting moiety may be a biological molecule, including but not limited to a fluorescent protein or fluorescent nucleic acid. In some embodiments, the signal-emitting moiety may be an inorganic moiety including Q-dots. In some embodiments, the signal-emitting moiety may be a moiety that operates through scattering, either elastic or inelastic scattering, such as nanoparticles and Surface Enhanced Raman Spectroscopy (SERS) reporters (e.g., 4-Mercaptobenzoic acid, 2,7-mercapto-4-methylcoumarin). In some embodiments, the signal-emitting moiety may be chemiluminescence/electrochemiluminescence emitters such as ruthenium complexes and luciferases. In some embodiments, the signal-emitting moiety generates an optical signal or an electromagnetic signal (across the entire electromagnetic spectrum).

As used herein, “flowable polymer” is a polymer monomer or macromer that is soluble or dispersible within a fluidic medium (e.g., a pre-polymer solution). The flowable polymer may be input into a microfluidic flow region and flow with other components of a fluidic medium therein.

As used herein, “photoinitiated polymer” refers to a polymer (or a monomeric molecule that can be used to generate the polymer) that upon exposure to light, is capable of crosslinking covalently, forming specific covalent bonds, changing regiochemistry around a rigidified chemical motif, or forming ion pairs which cause a change in physical state, and thereby forming a polymer network. In some instances, a photoinitiated polymer may include a polymer segment bound to one or more chemical moieties capable of crosslinking covalently, forming specific covalent bonds, changing regiochemistry around a rigidified chemical motif, or forming ion pairs which cause a change in physical state. In some instances, a photoinitiated polymer may require a photoactivatable radical initiator to initiate formation of the polymer network (e.g., via polymerization of the polymer).

As used herein, “antibody” refers to an immunoglobulin (Ig) and includes both polyclonal and monoclonal antibodies; primatized (e.g., humanized); murine; mouse-human; mouse-primate; and chimeric; and may be an intact molecule, a fragment thereof (such as scFv, Fv, Fd, Fab, Fab′ and F(ab)′2 fragments), or multimers or aggregates of intact molecules and/or fragments; and may occur in nature or be produced, e.g., by immunization, synthesis or genetic engineering. An “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′2, scFv, light chain variable region (VL), heavy chain variable region (VH), and combinations thereof.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result.

The phrase “substantially no flow” refers to a rate of flow of a fluidic medium that, averaged over time, is less than the rate of diffusion of components of a material (e.g., an analyte of interest) into or within the fluidic medium. The rate of diffusion of components of such a material can depend on, for example, temperature, the size of the components, and the strength of interactions between the components and the fluidic medium.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the microfluidic device.

As used herein, a “flow path” refers to one or more fluidically connected circuit elements (e.g. channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

As used herein, an “isolation region” refers to a region within a microfluidic device that is configured to hold a micro-object such that the micro-object is not drawn away from the region as a result of fluid flowing through the microfluidic device. Depending upon context, the term “isolation region” can further refer to the structures that define the region, which can include a base/substrate, walls (e.g., made from microfluidic circuit material), and a cover.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

The capability of biological micro-objects (e.g., biological cells) to produce specific biological materials (e.g., proteins, such as antibodies) can be assayed in such a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

As used herein, the term “transparent” refers to a material which allows visible light to pass through without substantially altering the light as is passes through.

As used herein, “brightfield” illumination and/or image refers to white light illumination of the microfluidic field of view from a broad-spectrum light source, where contrast is formed by absorbance of light by objects in the field of view.

As used herein, “structured light” is projected light which illuminates a portion of a surface of a device without illuminating an adjacent portion of the surface. Structured light is typically generated by a structured light modulator, such as a digital mirror device (DMD), a microshutter array system (MSA), a liquid crystal display (LCD), or the like. Structured light may be corrected for surface irregularities and for irregularities associated with the light projection itself, e.g., image fall-off at the edge of an illuminated field.

As used herein, the “clear aperture” of a lens (or lens assembly) is the diameter or size of the portion of the lens (or lens assembly) that can be used for its intended purpose. In some instances, the clear aperture can be substantially equal to the physical diameter of the lens (or lens assembly). However, owing to manufacturing constraints, it can be difficult to produce a clear aperture equal to the actual physical diameter of the lens (or lens assembly).

As used herein, the term “active area” refers to the portion of an image sensor or structured light modulator that can be used, respectively, to image or provide structured light to a field of view in a particular optical apparatus. The active area is subject to constraints of the optical apparatus, such as the aperture stop of the light path within the optical apparatus. Although the active area corresponds to a two-dimensional surface, the measurement of active area typically corresponds to the length of a diagonal line through opposing corners of a square having the same area.

As used herein, an “image light beam” is an electromagnetic wave that is reflected or emitted from a device surface, a micro-object, or a fluidic medium that is being viewed by an optical apparatus. The device can be any microfluidic device as described herein. The micro-object and the fluidic medium can be located within such a microfluidic device.

As used herein, the term “cell” is used interchangeably with the term “biological cell.” Non-limiting examples of biological cells include: eukaryotic cells, plant cells, animal cells, such as mammalian cells, reptilian cells, avian cells, fish cells, or the like; prokaryotic cells, bacterial cells, fungal cells, protozoan cells, or the like; cells dissociated from a tissue, such as muscle, cartilage, fat, skin, liver, or lung cells, neurons, glial cells, and the like; immunological cells, such as T cells, B cells, plasma cells, natural killer cells, macrophages, and the like; embryos (e.g., zygotes), germ cells, such as oocytes, ova, and sperm cells, and the like; fusion cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a pig, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the terms “maintaining a cell” and “maintaining cells” refer to providing an environment comprising both fluidic and gaseous components and, optionally a surface, that provides the conditions necessary to keep the cells viable and/or expanding.

As used herein, the term “expanding” when referring to cells, refers to increasing in cell number.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein, “capture moiety” is a chemical or biological species, functionality, or motif that provides a recognition site for a micro-object. A selected class of micro-objects may recognize the in situ-generated capture moiety and may bind or have an affinity for the in situ-generated capture moiety. Non-limiting examples include antigens, antibodies, and cell surface binding motifs.

As used herein, “antibody” refers to an immunoglobulin (Ig) and includes both polyclonal and monoclonal antibodies; multichain antibodies, such as IgG, IgM, IgA, IgE, and IgD antibodies; single chain antibodies, such as camelid antibodies; mammalian antibodies, including primate antibodies (e.g., human), rodent antibodies (e.g., mouse, rat, guinea pig, hamster, and the like), lagomorph antibodies (e.g., rabbit), ungulate antibodies (e.g., cow, pig, horse, donkey, camel, and the like), and canidae antibodies (e.g., dog); primatized (e.g., humanized) antibodies; chimeric antibodies, such as mouse-human, mouse-primate antibodies, or the like; and may be an intact molecule or a fragment thereof (such as a light chain variable region (VL), heavy chain variable region (VH), scFv, Fv, Fd, Fab, Fab′ and F(ab)′2 fragments), or multimers or aggregates of intact molecules and/or fragments; and may occur in nature or be produced, e.g., by immunization, synthesis or genetic engineering. An “antibody fragment,” as used herein, refers to fragments, derived from or related to an antibody, which bind antigen. In some embodiments, antibody fragments may be derivatized to exhibit structural features that facilitate clearance and uptake, e.g., by the incorporation of galactose residues. The capability of biological micro-objects (e.g., biological cells) to produce specific biological materials (e.g., proteins, such as antibodies) can be assayed in such a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., mammalian cells, such as human cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

An antigen, as referred to herein, is a molecule or portion thereof that can bind with specificity to another molecule, such as an Ag-specific receptor. An antigen may be any portion of a molecule, such as a conformational epitope or a linear molecular fragment, and often can be recognized by highly variable antigen receptors (B-cell receptor or T-cell receptor) of the adaptive immune system. An antigen may include a peptide, polysaccharide, or lipid. An antigen may be characterized by its ability to bind to an antibody's variable Fab region. Different antibodies have the potential to discriminate among different epitopes present on the antigen surface, the structure of which may be modulated by the presence of a hapten, which may be a small molecule.

The capability of biological micro-objects (e.g., biological cells) to produce specific biological materials (e.g., proteins, such as antibodies) can be assayed in a microfluidic device. In a specific embodiment of an assay, sample material comprising biological micro-objects (e.g., cells) to be assayed for production of an analyte of interest can be loaded into a swept region of the microfluidic device. Ones of the biological micro-objects (e.g., biological cells) can be selected for particular characteristics and disposed in unswept regions. The remaining sample material can then be flowed out of the swept region and an assay material flowed into the swept region. Because the selected biological micro-objects are in unswept regions, the selected biological micro-objects are not substantially affected by the flowing out of the remaining sample material or the flowing in of the assay material. The selected biological micro-objects can be allowed to produce the analyte of interest, which can diffuse from the unswept regions into the swept region, where the analyte of interest can react with the assay material to produce localized detectable reactions, each of which can be correlated to a particular unswept region. Any unswept region associated with a detected reaction can be analyzed to determine which, if any, of the biological micro-objects in the unswept region are sufficient producers of the analyte of interest.

### II. Microfluidic Devices and Systems for Operating and Observing Such Devices

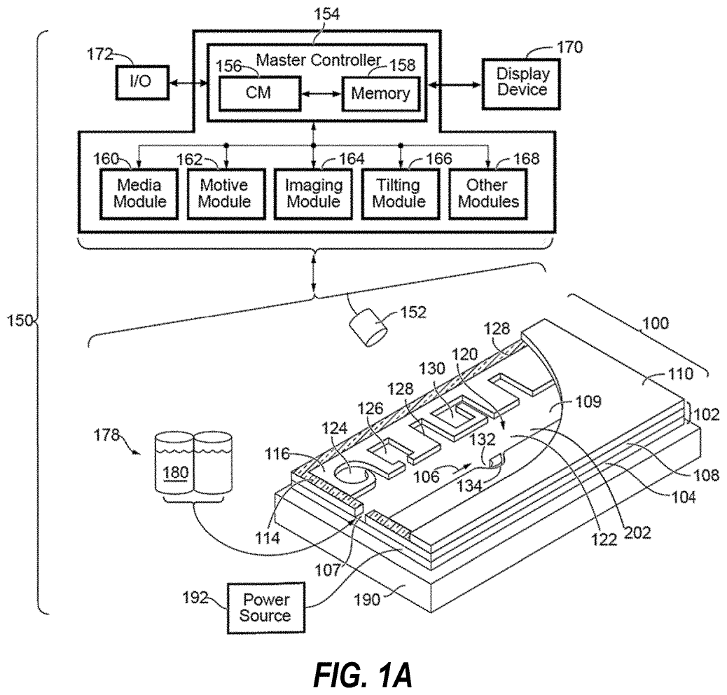

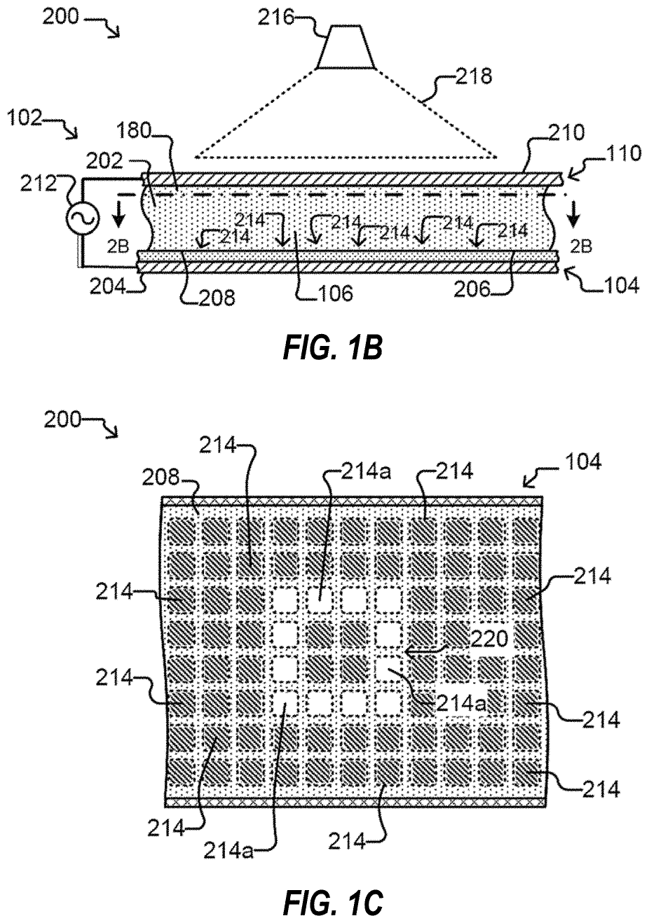

FIG. 1A illustrates an example of a microfluidic device 100 and a system 150 which can be used to assay binding affinity between a first molecule and a second molecule. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120. Although a single microfluidic circuit 120 is illustrated in FIG. 1A, suitable microfluidic devices can include a plurality (e.g., 2 or 3) of such microfluidic circuits. Regardless, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings in fluidic communication with flow path 106. In some embodiments of the device of FIG. 1A, the sequestration pens may have only a single opening in fluidic communication with the flow path 106. As discussed further below, the microfluidic sequestration pens comprise various features and structures that have been optimized for retaining micro-objects in the microfluidic device, such as microfluidic device 100, even when a medium 180 is flowing through the flow path 106. Before turning to the foregoing, however, a brief description of microfluidic device 100 and system 150 is provided.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers, pens, traps, and the like. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material.

The microfluidic circuit material 116 can be patterned with cavities or the like to define circuit elements and interconnections of the microfluidic circuit 120. The microfluidic circuit material 116 can comprise a flexible material, such as a flexible polymer (e.g. rubber, plastic, elastomer, silicone, polydimethylsiloxane (“PDMS”), or the like), which can be gas permeable. Other examples of materials that can compose microfluidic circuit material 116 include molded glass, an etchable material such as silicone (e.g. photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. 2012/0325665 (Chiou et al.), the contents of which are incorporated herein by reference. In some embodiments, the cover 110 can be modified (e.g., by conditioning all or part of a surface that faces inward toward the microfluidic circuit 120) to support cell adhesion, viability and/or growth. The modification may include a coating of a synthetic or natural polymer. In some embodiments, the cover 110 and/or the support structure 104 can be transparent to light. The cover 110 may also include at least one material that is gas permeable (e.g., PDMS or PPS).

FIG. 1A also shows a system 150 for operating and controlling microfluidic devices, such as microfluidic device 100. System 150 includes an electrical power source 192, an imaging device (which may be incorporated within imaging module 164, where the imaging device is not illustrated in FIG. 1A, per se), and a tilting device 190 (part of tilting module 166, where device 190 is not illustrated in FIG. 1A).

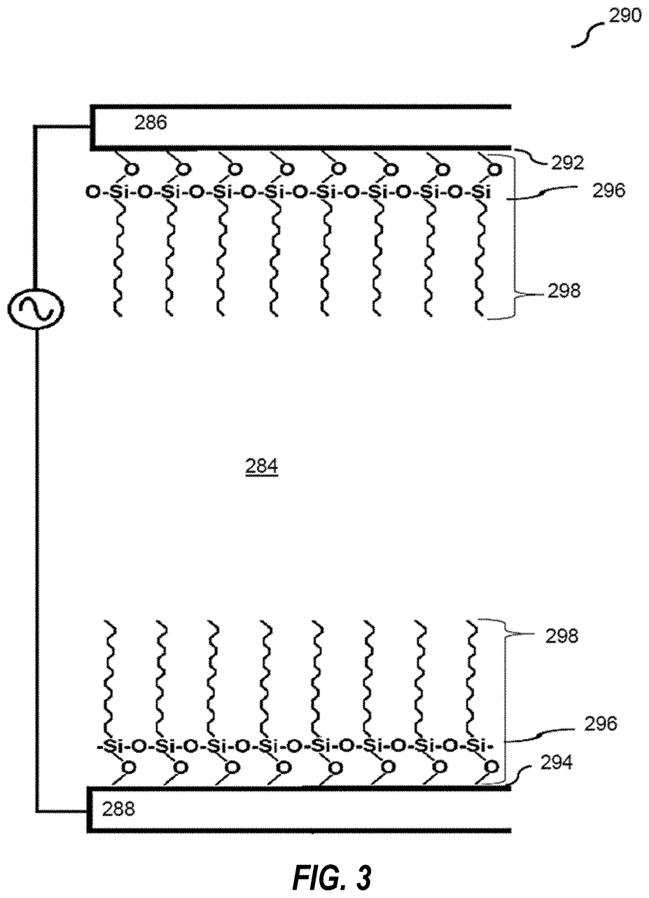

The electrical power source 192 can provide electric power to the microfluidic device 100 and/or tilting device 190, providing biasing voltages or currents as needed. The electrical power source 192 can, for example, comprise one or more alternating current (AC) and/or direct current (DC) voltage or current sources. The imaging device 194 (part of imaging module 164, discussed below) can comprise a device, such as a digital camera, for capturing images inside microfluidic circuit 120. In some instances, the imaging device 194 further comprises a detector having a fast frame rate and/or high sensitivity (e.g. for low light applications). The imaging device 194 can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g. a high pressure mercury lamp) or a Xenon arc lamp. As discussed with respect to FIG. 3B, the imaging device 194 may further include a microscope (or an optical train), which may or may not include an eyepiece.

System 150 further comprises a tilting device 190 (part of tilting module 166, discussed below) configured to rotate a microfluidic device 100 about one or more axes of rotation. In some embodiments, the tilting device 190 is configured to support and/or hold the enclosure 102 comprising the microfluidic circuit 120 about at least one axis such that the microfluidic device 100 (and thus the microfluidic circuit 120) can be held in a level orientation (i.e. at 0° relative to x- and y-axes), a vertical orientation (i.e. at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, the tilting device 190 can tilt the microfluidic device 100 at 0.1°, 02°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. The level orientation (and thus the x- and y-axes) is defined as normal to a vertical axis defined by the force of gravity. The tilting device can also tilt the microfluidic device 100 (and the microfluidic circuit 120) to any degree greater than 90° relative to the x-axis and/or y-axis, or tilt the microfluidic device 100 (and the microfluidic circuit 120) 180° relative to the x-axis or the y-axis in order to fully invert the microfluidic device 100 (and the microfluidic circuit 120). Similarly, in some embodiments, the tilting device 190 tilts the microfluidic device 100 (and the microfluidic circuit 120) about an axis of rotation defined by flow path 106 or some other portion of microfluidic circuit 120.

In some instances, the microfluidic device 100 is tilted into a vertical orientation such that the flow path 106 is positioned above or below one or more sequestration pens. The term “above” as used herein denotes that the flow path 106 is positioned higher than the one or more sequestration pens on a vertical axis defined by the force of gravity (i.e. an object in a sequestration pen above a flow path 106 would have a higher gravitational potential energy than an object in the flow path). The term “below” as used herein denotes that the flow path 106 is positioned lower than the one or more sequestration pens on a vertical axis defined by the force of gravity (i.e. an object in a sequestration pen below a flow path 106 would have a lower gravitational potential energy than an object in the flow path).

In some instances, the tilting device 190 tilts the microfluidic device 100 about an axis that is parallel to the flow path 106. Moreover, the microfluidic device 100 can be tilted to an angle of less than 90° such that the flow path 106 is located above or below one or more sequestration pens without being located directly above or below the sequestration pens. In other instances, the tilting device 190 tilts the microfluidic device 100 about an axis perpendicular to the flow path 106. In still other instances, the tilting device 190 tilts the microfluidic device 100 about an axis that is neither parallel nor perpendicular to the flow path 106.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device 194 (e.g., a camera, microscope, light source or any combination thereof) for capturing images (e.g., digital images), and a tilting module 166 for controlling a tilting device 190. The control equipment 152 can also include other modules 168 for controlling, monitoring, or performing other functions with respect to the microfluidic device 100. As shown, the equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, tilting module 166, and/or other modules 168 configured as discussed above. Similarly, the master controller 154, media module 160, motive module 162, imaging module 164, tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. For example, in some embodiments media module 160 stops the flow of media 180 in the flow path 106 and through the enclosure 102 prior to the tilting module 166 causing the tilting device 190 to tilt the microfluidic device 100 to a desired angle of incline.

The motive module 162 can be configured to control selection, trapping, and movement of micro-objects (not shown) in the microfluidic circuit 120. As discussed below with respect to FIGS. 1B and 1C, the enclosure 102 can comprise a dielectrophoresis (DEP), optoelectronic tweezers (OET) and/or opto-electrowetting (OEW) configuration (not shown in FIG. 1A), and the motive module 162 can control the activation of electrodes and/or transistors (e.g., phototransistors) to select and move micro-objects (not shown) and/or droplets of medium (not shown) in the flow path 106 and/or sequestration pens 124, 126, 128, 130.

The imaging module 164 can control the imaging device 194. For example, the imaging module 164 can receive and process image data from the imaging device 194. Image data from the imaging device 194 can comprise any type of information captured by the imaging device 194 (e.g., the presence or absence of micro-objects, droplets of medium, accumulation of label, such as fluorescent label, etc.). Using the information captured by the imaging device 194, the imaging module 164 can further calculate the position of objects (e.g., micro-objects, droplets of medium) and/or the rate of motion of such objects within the microfluidic device 100.

The tilting module 166 can control the tilting motions of tilting device 190. Alternatively, or in addition, the tilting module 166 can control the tilting rate and timing to optimize transfer of micro-objects to the one or more sequestration pens via gravitational forces. The tilting module 166 is communicatively coupled with the imaging module 164 to receive data describing the motion of micro-objects and/or droplets of medium in the microfluidic circuit 120. Using this data, the tilting module 166 may adjust the tilt of the microfluidic circuit 120 in order to adjust the rate at which micro-objects and/or droplets of medium move in the microfluidic circuit 120. The tilting module 166 may also use this data to iteratively adjust the position of a micro-object and/or droplet of medium in the microfluidic circuit 120.

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the pen to the microfluidic channel 122 is oriented at an angle to the flow 106 of fluidic medium 180 such that flow 106 is not directed into the pens. The flow may be tangential or orthogonal to the plane of the opening of the pen. In some instances, pens 124, 126, 128, 130 are configured to physically corral one or more micro-objects within the microfluidic circuit 120. Sequestration pens in accordance with the present disclosure can comprise various shapes, surfaces and features that are optimized for use with DEP, OET, OEW, fluid flow, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing micro-objects, including biological cells and other micro-objects such as beads. In some embodiments, the microfluidic circuit 120 comprises a plurality of identical microfluidic sequestration pens.

In the embodiment illustrated in FIG. 1A, a single channel 122 and flow path 106 is shown. However, other embodiments may contain multiple channels 122, each configured to comprise a flow path 106. The microfluidic circuit 120 further comprises an inlet valve or port 107 in fluid communication with the flow path 106 and fluidic medium 180, whereby fluidic medium 180 can access channel 122 via the inlet port 107. In some instances, the flow path 106 comprises a single path. In some instances, the single path is arranged in a zigzag pattern whereby the flow path 106 travels across the microfluidic device 100 two or more times in alternating directions.

In some instances, microfluidic circuit 120 comprises a plurality of parallel channels 122 and flow paths 106, wherein the fluidic medium 180 within each flow path 106 flows in the same direction. In some instances, the fluidic medium within each flow path 106 flows in at least one of a forward or reverse direction. In some instances, a plurality of sequestration pens is configured (e.g., relative to a channel 122) such that the sequestration pens can be loaded with target micro-objects in parallel.

In some embodiments, microfluidic circuit 120 further comprises one or more micro-object traps 132. The traps 132 are generally formed in a wall forming the boundary of a channel 122, and may be positioned opposite an opening of one or more of the microfluidic sequestration pens 124, 126, 128, 130. In some embodiments, the traps 132 are configured to receive or capture a single micro-object from the flow path 106. In some embodiments, the traps 132 are configured to receive or capture a plurality of micro-objects from the flow path 106. In some instances, the traps 132 comprise a volume approximately equal to the volume of a single target micro-object.

The traps 132 may further comprise an opening which is configured to assist the flow of targeted micro-objects into the traps 132. In some instances, the traps 132 comprise an opening having a height and width that is approximately equal to the dimensions of a single target micro-object, whereby larger micro-objects are prevented from entering into the micro-object trap. The traps 132 may further comprise other features configured to assist in retention of targeted micro-objects within the trap 132. In some instances, the trap 132 is aligned with and situated on the opposite side of a channel 122 relative to the opening of a microfluidic sequestration pen, such that upon tilting the microfluidic device 100 about an axis parallel to the microfluidic channel 122, the trapped micro-object exits the trap 132 at a trajectory that causes the micro-object to fall into the opening of the sequestration pen. In some instances, the trap 132 comprises a side passage 134 that is smaller than the target micro-object in order to facilitate flow through the trap 132 and thereby increase the likelihood of capturing a micro-object in the trap 132.

In some embodiments, dielectrophoretic (DEP) forces are applied across the fluidic medium 180 (e.g., in the flow path and/or in the sequestration pens) via one or more electrodes (not shown) to manipulate, transport, separate and sort micro-objects located therein. For example, in some embodiments, DEP forces are applied to one or more portions of microfluidic circuit 120 in order to transfer a single micro-object from the flow path 106 into a desired microfluidic sequestration pen. In some embodiments, DEP forces are used to prevent a micro-object within a sequestration pen (e.g., sequestration pen 124, 126, 128, or 130) from being displaced therefrom. Further, in some embodiments, DEP forces are used to selectively remove a micro-object from a sequestration pen that was previously collected in accordance with the embodiments of the current disclosure. In some embodiments, the DEP forces comprise optoelectronic tweezer (OET) forces.

In other embodiments, optoelectrowetting (OEW) forces are applied to one or more positions in the support structure 104 (and/or the cover 110) of the microfluidic device 100 (e.g., positions helping to define the flow path and/or the sequestration pens) via one or more electrodes (not shown) to manipulate, transport, separate and sort droplets located in the microfluidic circuit 120. For example, in some embodiments, OEW forces are applied to one or more positions in the support structure 104 (and/or the cover 110) in order to transfer a single droplet from the flow path 106 into a desired microfluidic sequestration pen. In some embodiments, OEW forces are used to prevent a droplet within a sequestration pen (e.g., sequestration pen 124, 126, 128, or 130) from being displaced therefrom. Further, in some embodiments, OEW forces are used to selectively remove a droplet from a sequestration pen that was previously collected in accordance with the embodiments of the current disclosure.

In some embodiments, DEP and/or OEW forces are combined with other forces, such as flow and/or gravitational force, so as to manipulate, transport, separate and sort micro-objects and/or droplets within the microfluidic circuit 120. For example, the enclosure 102 can be tilted (e.g., by tilting device 190) to position the flow path 106 and micro-objects located therein above the microfluidic sequestration pens, and the force of gravity can transport the micro-objects and/or droplets into the pens. In some embodiments, the DEP and/or OEW forces can be applied prior to the other forces. In other embodiments, the DEP and/or OEW forces can be applied after the other forces. In still other instances, the DEP and/or OEW forces can be applied at the same time as the other forces or in an alternating manner with the other forces.

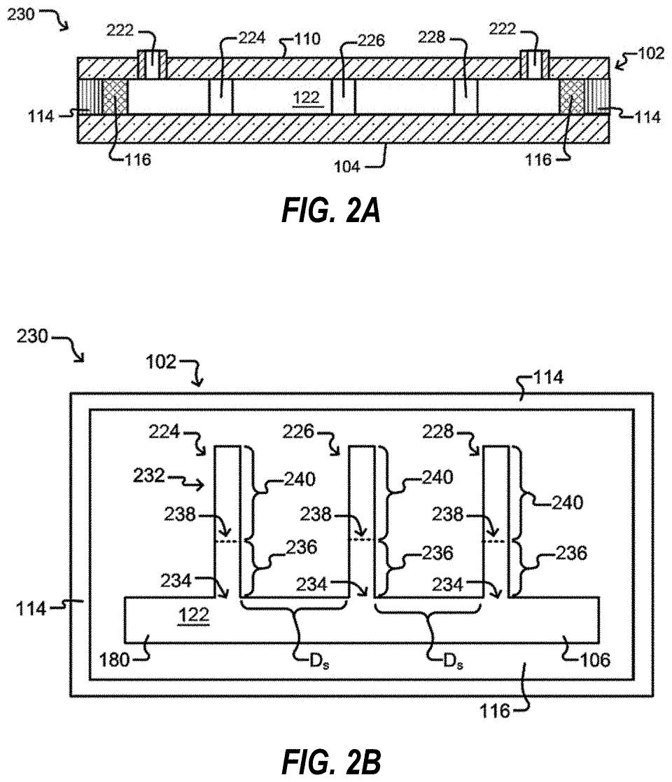

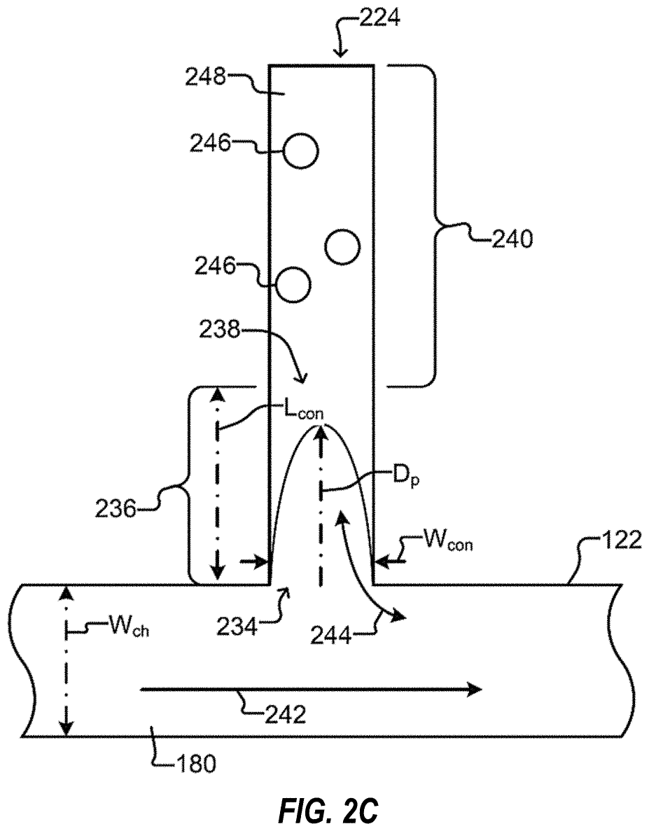

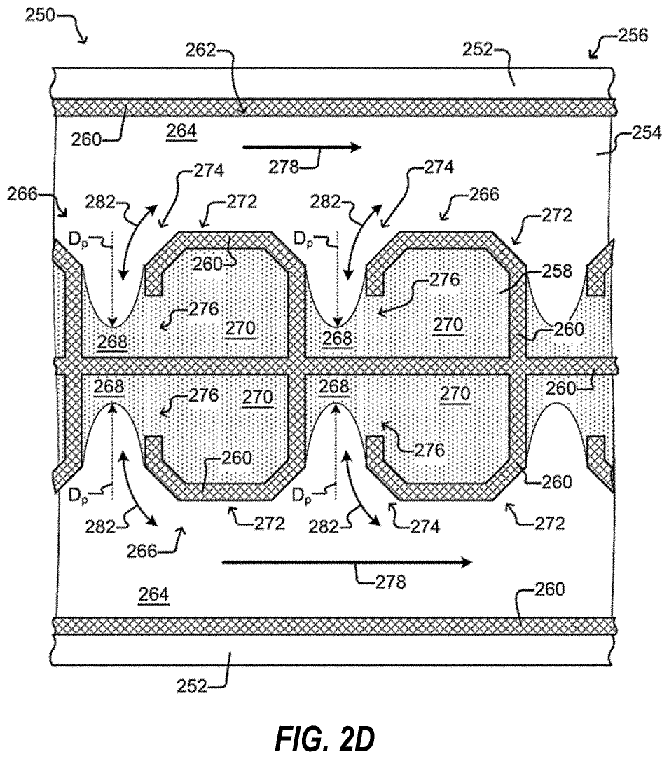

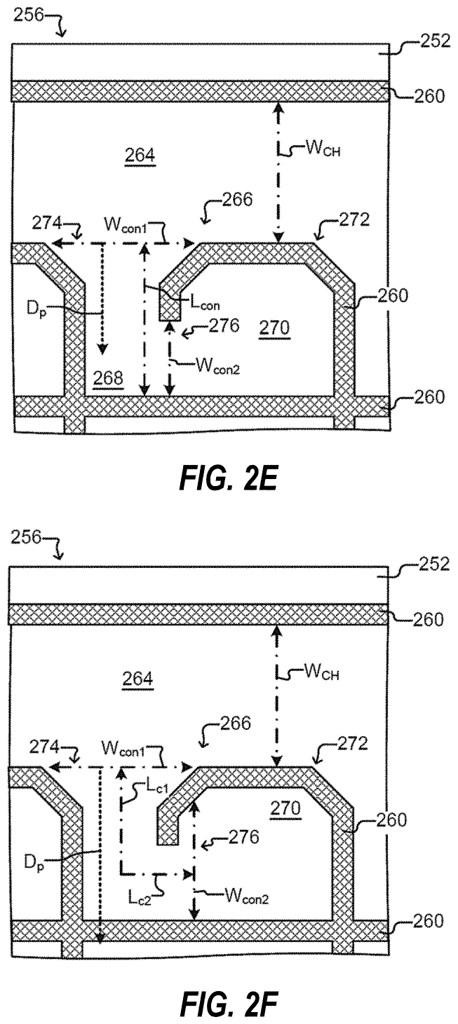

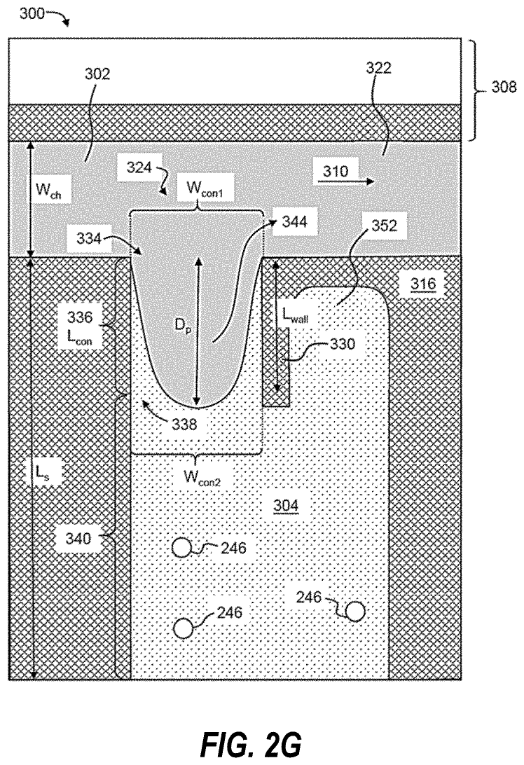

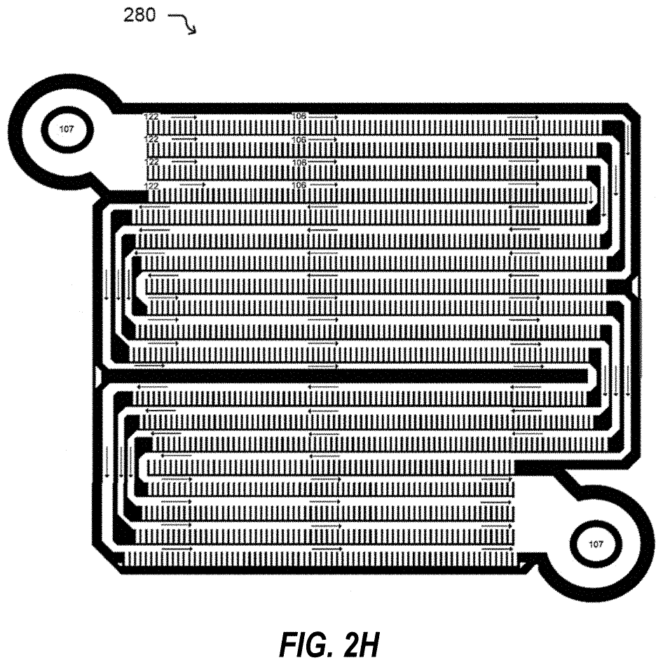

FIGS. 1B, 1C, and 2A-2H illustrates various embodiments of microfluidic devices that can be used in the practice of the embodiments of the present disclosure. FIG. 1B depicts an embodiment in which the microfluidic device 200 is configured as an optically-actuated electrokinetic device. A variety of optically-actuated electrokinetic devices are known in the art, including devices having an optoelectronic tweezer (OET) configuration and devices having an opto-electrowetting (OEW) configuration. Examples of suitable OET configurations are illustrated in the following U.S. patent documents, each of which is incorporated herein by reference in its entirety: U.S. Pat. No. RE 44,711 (Wu et al.) (originally issued as U.S. Pat. No. 7,612,355); and U.S. Pat. No. 7,956,339 (Ohta et al.). Examples of OEW configurations are illustrated in U.S. Pat. No. 6,958,132 (Chiou et al.) and U.S. Patent Application Publication No. 2012/0024708 (Chiou et al.), both of which are incorporated by reference herein in their entirety. Yet another example of an optically-actuated electrokinetic device includes a combined OET/OEW configuration, examples of which are shown in U.S. Patent Publication Nos. 20150306598 (Khandros et al.) and 20150306599 (Khandros et al.) and their corresponding PCT Publications WO2015/164846 and WO2015/164847, all of which are incorporated herein by reference in their entirety.

Examples of microfluidic devices having pens in which micro-objects can be placed, cultured, and/or monitored have been described, for example, in US 2014/0116881 (application Ser. No. 14/060,117, filed Oct. 22, 2013), US 2015/0151298 (application Ser. No. 14/520,568, filed Oct. 22, 2014), and US 2015/0165436 (application Ser. No. 14/521,447, filed Oct. 22, 2014), each of which is incorporated herein by reference in its entirety. U.S. application Ser. Nos. 14/520,568 and 14/521,447 also describe exemplary methods of analyzing secretions of cells cultured in a microfluidic device. Each of the foregoing applications further describes microfluidic devices configured to produce dielectrophoretic (DEP) forces, such as optoelectronic tweezers (OET) or configured to provide opto-electro wetting (OEW). For example, the optoelectronic tweezers device illustrated in FIG. 2 of US 2014/0116881 is an example of a device that can be utilized in embodiments of the present disclosure to select and move an individual biological micro-object or a group of biological micro-objects.

### III. Microfluidic Device Motive Configurations

As described above, the control and monitoring equipment of the system can comprise a motive module for selecting and moving objects, such as micro-objects or droplets, in the microfluidic circuit of a microfluidic device. The microfluidic device can have a variety of motive configurations, depending upon the type of object being moved and other considerations. For example, a dielectrophoresis (DEP) configuration can be utilized to select and move micro-objects in the microfluidic circuit. Thus, the support structure 104 and/or cover 110 of the microfluidic device 100 can comprise a DEP configuration for selectively inducing DEP forces on micro-objects in a fluidic medium 180 in the microfluidic circuit 120 and thereby select, capture, and/or move individual micro-objects or groups of micro-objects. Alternatively, the support structure 104 and/or cover 110 of the microfluidic device 100 can comprise an electrowetting (EW) configuration for selectively inducing EW forces on droplets in a fluidic medium 180 in the microfluidic circuit 120 and thereby select, capture, and/or move individual droplets or groups of droplets.

One example of a microfluidic device 200 comprising a DEP configuration is illustrated in FIGS. 1B and 1C. While for purposes of simplicity FIGS. 1B and 1C show a side cross-sectional view and a top cross-sectional view, respectively, of a portion of an enclosure 102 of the microfluidic device 200 having a region/chamber 202, it should be understood that the region/chamber 202 may be part of a fluidic circuit element having a more detailed structure, such as a growth chamber, a sequestration pen, a flow region, or a flow channel. Furthermore, the microfluidic device 200 may include other fluidic circuit elements. For example, the microfluidic device 200 can include a plurality of growth chambers or sequestration pens and/or one or more flow regions or flow channels, such as those described herein with respect to microfluidic device 100. A DEP configuration may be incorporated into any such fluidic circuit elements of the microfluidic device 200, or select portions thereof. It should be further appreciated that any of the above or below described microfluidic device components and system components may be incorporated in and/or used in combination with the microfluidic device 200. For example, system 150 including control and monitoring equipment 152, described above, may be used with microfluidic device 200, including one or more of the media module 160, motive module 162, imaging module 164, tilting module 166, and other modules 168.

As seen in FIG. 1B, the microfluidic device 200 includes a support structure 104 having a bottom electrode 204 and an electrode activation substrate 206 overlying the bottom electrode 204, and a cover 110 having a top electrode 210, with the top electrode 210 spaced apart from the bottom electrode 204. The top electrode 210 and the electrode activation substrate 206 define opposing surfaces of the region/chamber 202. A medium 180 contained in the region/chamber 202 thus provides a resistive connection between the top electrode 210 and the electrode activation substrate 206. A power source 212 configured to be connected to the bottom electrode 204 and the top electrode 210 and create a biasing voltage between the electrodes, as required for the generation of DEP forces in the region/chamber 202, is also shown. The power source 212 can be, for example, an alternating current (AC) power source.

In certain embodiments, the microfluidic device 200 illustrated in FIGS. 1B and 1C can have an optically-actuated DEP configuration. Accordingly, changing patterns of light 218 from the light source 216, which may be controlled by the motive module 162, can selectively activate and deactivate changing patterns of DEP electrodes at regions 214 of the inner surface 208 of the electrode activation substrate 206. (Hereinafter the regions 214 of a microfluidic device having a DEP configuration are referred to as “DEP electrode regions.”) As illustrated in FIG. 1C, a light pattern 218 directed onto the inner surface 208 of the electrode activation substrate 206 can illuminate select DEP electrode regions 214a (shown in white) in a pattern, such as a square. The non-illuminated DEP electrode regions 214 (cross-hatched) are hereinafter referred to as “dark” DEP electrode regions 214. The relative electrical impedance through the DEP electrode activation substrate 206 (i.e., from the bottom electrode 204 up to the inner surface 208 of the electrode activation substrate 206 which interfaces with the medium 180 in the flow region 106) is greater than the relative electrical impedance through the medium 180 in the region/chamber 202 (i.e., from the inner surface 208 of the electrode activation substrate 206 to the top electrode 210 of the cover 110) at each dark DEP electrode region 214. An illuminated DEP electrode region 214a, however, exhibits a reduced relative impedance through the electrode activation substrate 206 that is less than the relative impedance through the medium 180 in the region/chamber 202 at each illuminated DEP electrode region 214a.

With the power source 212 activated, the foregoing DEP configuration creates an electric field gradient in the fluidic medium 180 between illuminated DEP electrode regions 214a and adjacent dark DEP electrode regions 214, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 214 at the inner surface 208 of the region/chamber 202 by changing light patterns 218 projected from a light source 216 into the microfluidic device 200. Whether the DEP forces attract or repel nearby micro-objects can depend on such parameters as the frequency of the power source 212 and the dielectric properties of the medium 180 and/or micro-objects (not shown).

The square pattern 220 of illuminated DEP electrode regions 214a illustrated in FIG. 1C is an example only. Any pattern of the DEP electrode regions 214 can be illuminated (and thereby activated) by the pattern of light 218 projected into the microfluidic device 200, and the pattern of illuminated/activated DEP electrode regions 214 can be repeatedly changed by changing or moving the light pattern 218.

In some embodiments, the electrode activation substrate 206 can comprise or consist of a photoconductive material. In such embodiments, the inner surface 208 of the electrode activation substrate 206 can be featureless. For example, the electrode activation substrate 206 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 214 can be created anywhere and in any pattern on the inner surface 208 of the electrode activation substrate 206, in accordance with the light pattern 218. The number and pattern of the DEP electrode regions 214 thus need not be fixed, but can correspond to the light pattern 218. Examples of microfluidic devices having a DEP configuration comprising a photoconductive layer such as discussed above have been described, for example, in U.S. Pat. No. RE 44,711 (Wu et al.) (originally issued as U.S. Pat. No. 7,612,355), the entire contents of which are incorporated herein by reference.

In other embodiments, the electrode activation substrate 206 can comprise a substrate comprising a plurality of doped layers, electrically insulating layers (or regions), and electrically conductive layers that form semiconductor integrated circuits, such as is known in semiconductor fields. For example, the electrode activation substrate 206 can comprise a plurality of phototransistors, including, for example, lateral bipolar phototransistors, each phototransistor corresponding to a DEP electrode region 214. Alternatively, the electrode activation substrate 206 can comprise electrodes (e.g., conductive metal electrodes) controlled by phototransistor switches, with each such electrode corresponding to a DEP electrode region 214. The electrode activation substrate 206 can include a pattern of such phototransistors or phototransistor-controlled electrodes. The pattern, for example, can be an array of substantially square phototransistors or phototransistor-controlled electrodes arranged in rows and columns, such as shown in FIG. 2B. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 214 at the inner surface 208 of the electrode activation substrate 206 and the bottom electrode 210, and those electrical connections (i.e., phototransistors or electrodes) can be selectively activated and deactivated by the light pattern 218. When not activated, each electrical connection can have high impedance such that the relative impedance through the electrode activation substrate 206 (i.e., from the bottom electrode 204 to the inner surface 208 of the electrode activation substrate 206 which interfaces with the medium 180 in the region/chamber 202) is greater than the relative impedance through the medium 180 (i.e., from the inner surface 208 of the electrode activation substrate 206 to the top electrode 210 of the cover 110) at the corresponding DEP electrode region 214. When activated by light in the light pattern 218, however, the relative impedance through the electrode activation substrate 206 is less than the relative impedance through the medium 180 at each illuminated DEP electrode region 214, thereby activating the DEP electrode at the corresponding DEP electrode region 214 as discussed above. DEP electrodes that attract or repel micro-objects (not shown) in the medium 180 can thus be selectively activated and deactivated at many different DEP electrode regions 214 at the inner surface 208 of the electrode activation substrate 206 in the region/chamber 202 in a manner determined by the light pattern 218.

Examples of microfluidic devices having electrode activation substrates that comprise phototransistors have been described, for example, in U.S. Pat. No. 7,956,339 (Ohta et al.) (see, e.g., device 300 illustrated in FIGS. 21 and 22, and descriptions thereof), the entire contents of which are incorporated herein by reference. Examples of microfluidic devices having electrode activation substrates that comprise electrodes controlled by phototransistor switches have been described, for example, in U.S. Patent Publication No. 2014/0124370 (Short et al.) (see, e.g., devices 200, 400, 500, 600, and 900 illustrated throughout the drawings, and descriptions thereof), the entire contents of which are incorporated herein by reference.

In some embodiments of a DEP configured microfluidic device, the top electrode 210 is part of a first wall (or cover 110) of the enclosure 102, and the electrode activation substrate 206 and bottom electrode 204 are part of a second wall (or support structure 104) of the enclosure 102. The region/chamber 202 can be between the first wall and the second wall. In other embodiments, the electrode 210 is part of the second wall (or support structure 104) and one or both of the electrode activation substrate 206 and/or the electrode 210 are part of the first wall (or cover 110). Moreover, the light source 216 can alternatively be used to illuminate the enclosure 102 from below.

With the microfluidic device 200 of FIGS. 1B-1C having a DEP configuration, the motive module 162 can select a micro-object (not shown) in the medium 180 in the region/chamber 202 by projecting a light pattern 218 into the microfluidic device 200 to activate a first set of one or more DEP electrodes at DEP electrode regions 214a of the inner surface 208 of the electrode activation substrate 206 in a pattern (e.g., square pattern 220) that surrounds and captures the micro-object. The motive module 162 can then move the in situ-generated captured micro-object by moving the light pattern 218 relative to the microfluidic device 200 to activate a second set of one or more DEP electrodes at DEP electrode regions 214. Alternatively, the microfluidic device 200 can be moved relative to the light pattern 218.

In other embodiments, the microfluidic device 200 can have a DEP configuration that does not rely upon light activation of DEP electrodes at the inner surface 208 of the electrode activation substrate 206. For example, the electrode activation substrate 206 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 214, thereby creating a net DEP force on a micro-object (not shown) in region/chamber 202 in the vicinity of the activated DEP electrodes. Depending on such characteristics as the frequency of the power source 212 and the dielectric properties of the medium (not shown) and/or micro-objects in the region/chamber 202, the DEP force can attract or repel a nearby micro-object. By selectively activating and deactivating a set of DEP electrodes (e.g., at a set of DEP electrodes regions 214 that forms a square pattern 220), one or more micro-objects in region/chamber 202 can be trapped and moved within the region/chamber 202. The motive module 162 in FIG. 1A can control such switches and thus activate and deactivate individual ones of the DEP electrodes to select, trap, and move particular micro-objects (not shown) around the region/chamber 202. Microfluidic devices having a DEP configuration that includes selectively addressable and energizable electrodes are known in the art and have been described, for example, in U.S. Pat. No. 6,294,063 (Becker et al.) and U.S. Pat. No. 6,942,776 (Medoro), the entire contents of which are incorporated herein by reference.

As yet another example, the microfluidic device 200 can have an electrowetting (EW) configuration, which can be in place of the DEP configuration or can be located in a portion of the microfluidic device 200 that is separate from the portion which has the DEP configuration. The EW configuration can be an opto-electrowetting configuration or an electrowetting on dielectric (EWOD) configuration, both of which are known in the art. In some EW configurations, the support structure 104 has an electrode activation substrate 206 sandwiched between a dielectric layer (not shown) and the bottom electrode 204. The dielectric layer can comprise a hydrophobic material and/or can be coated with a hydrophobic material, as described below. For microfluidic devices 200 that have an EW configuration, the inner surface 208 of the support structure 104 is the inner surface of the dielectric layer or its hydrophobic coating.

The dielectric layer (not shown) can comprise one or more oxide layers, and can have a thickness of about 50 nm to about 250 nm (e.g., about 125 nm to about 175 nm). In certain embodiments, the dielectric layer may comprise a layer of oxide, such as a metal oxide (e.g., aluminum oxide or hafnium oxide). In certain embodiments, the dielectric layer can comprise a dielectric material other than a metal oxide, such as silicon oxide or a nitride. Regardless of the exact composition and thickness, the dielectric layer can have an impedance of about 10 kOhms to about 50 kOhms.

In some embodiments, the surface of the dielectric layer that faces inward toward region/chamber 202 is coated with a hydrophobic material. The hydrophobic material can comprise, for example, fluorinated carbon molecules. Examples of fluorinated carbon molecules include perfluoro-polymers such as polytetrafluoroethylene (e.g., TEFLON®) or poly(2,3-difluoromethylenyl-perfluorotetrahydrofuran) (e.g., CYTOP™). Molecules that make up the hydrophobic material can be covalently bonded to the surface of the dielectric layer. For example, molecules of the hydrophobic material can be covalently bound to the surface of the dielectric layer by means of a linker such as a siloxane group, a phosphonic acid group, or a thiol group. Thus, in some embodiments, the hydrophobic material can comprise alkyl-terminated siloxane, alkyl-termination phosphonic acid, or alkyl-terminated thiol. The alkyl group can be long-chain hydrocarbons (e.g., having a chain of at least 10 carbons, or at least 16, 18, 20, 22, or more carbons). Alternatively, fluorinated (or perfluorinated) carbon chains can be used in place of the alkyl groups. Thus, for example, the hydrophobic material can comprise fluoroalkyl-terminated siloxane, fluoroalkyl-terminated phosphonic acid, or fluoroalkyl-terminated thiol. In some embodiments, the hydrophobic coating has a thickness of about 10 nm to about 50 nm. In other embodiments, the hydrophobic coating has a thickness of less than 10 nm (e.g., less than 5 nm, or about 1.5 to 3.0 nm).

In some embodiments, the cover 110 of a microfluidic device 200 having an electrowetting configuration is coated with a hydrophobic material (not shown) as well. The hydrophobic material can be the same hydrophobic material used to coat the dielectric layer of the support structure 104, and the hydrophobic coating can have a thickness that is substantially the same as the thickness of the hydrophobic coating on the dielectric layer of the support structure 104. Moreover, the cover 110 can comprise an electrode activation substrate 206 sandwiched between a dielectric layer and the top electrode 210, in the manner of the support structure 104. The electrode activation substrate 206 and the dielectric layer of the cover 110 can have the same composition and/or dimensions as the electrode activation substrate 206 and the dielectric layer of the support structure 104. Thus, the microfluidic device 200 can have two electrowetting surfaces.

In some embodiments, the electrode activation substrate 206 can comprise a photoconductive material, such as described above. Accordingly, in certain embodiments, the electrode activation substrate 206 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. Alternatively, the electrode activation substrate 206 can comprise electrodes (e.g., conductive metal electrodes) controlled by phototransistor switches, as described above. Microfluidic devices having an opto-electrowetting configuration are known in the art and/or can be constructed with electrode activation substrates known in the art. For example, U.S. Pat. No. 6,958,132 (Chiou et al.), the entire contents of which are incorporated herein by reference, discloses opto-electrowetting configurations having a photoconductive material such as a-Si:H, while U.S. Patent Publication No. 2014/0124370 (Short et al.), referenced above, discloses electrode activation substrates having electrodes controlled by phototransistor switches.

The microfluidic device 200 thus can have an opto-electrowetting configuration, and light patterns 218 can be used to activate photoconductive EW regions or photoresponsive EW electrodes in the electrode activation substrate 206. Such activated EW regions or EW electrodes of the electrode activation substrate 206 can generate an electrowetting force at the inner surface 208 of the support structure 104 (i.e., the inner surface of the overlaying dielectric layer or its hydrophobic coating). By changing the light patterns 218 (or moving microfluidic device 200 relative to the light source 216) incident on the electrode activation substrate 206, droplets (e.g., containing an aqueous medium, solution, or solvent) contacting the inner surface 208 of the support structure 104 can be moved through an immiscible fluid (e.g., an oil medium) present in the region/chamber 202.

In other embodiments, microfluidic devices 200 can have an EWOD configuration, and the electrode activation substrate 206 can comprise selectively addressable and energizable electrodes that do not rely upon light for activation. The electrode activation substrate 206 thus can include a pattern of such electrowetting (EW) electrodes. The pattern, for example, can be an array of substantially square EW electrodes arranged in rows and columns, such as shown in FIG. 2B. Alternatively, the pattern can be an array of substantially hexagonal EW electrodes that form a hexagonal lattice. Regardless of the pattern, the EW electrodes can be selectively activated (or deactivated) by electrical switches (e.g., transistor switches in a semiconductor substrate). By selectively activating and deactivating EW electrodes in the electrode activation substrate 206, droplets (not shown) contacting the inner surface 208 of the overlaying dielectric layer or its hydrophobic coating can be moved within the region/chamber 202. The motive module 162 in FIG. 1A can control such switches and thus activate and deactivate individual EW electrodes to select and move particular droplets around region/chamber 202. Microfluidic devices having a EWOD configuration with selectively addressable and energizable electrodes are known in the art and have been described, for example, in U.S. Pat. No. 8,685,344 (Sundarsan et al.), the entire contents of which are incorporated herein by reference.

Regardless of the configuration of the microfluidic device 200, a power source 212 can be used to provide a potential (e.g., an AC voltage potential) that powers the electrical circuits of the microfluidic device 200. The power source 212 can be the same as, or a component of, the power source 192 referenced in FIG. 1. Power source 212 can be configured to provide an AC voltage and/or current to the top electrode 210 and the bottom electrode 204. For an AC voltage, the power source 212 can provide a frequency range and an average or peak power (e.g., voltage or current) range sufficient to generate net DEP forces (or electrowetting forces) strong enough to trap and move individual micro-objects (not shown) in the region/chamber 202, as discussed above, and/or to change the wetting properties of the inner surface 208 of the support structure 104 (i.e., the dielectric layer and/or the hydrophobic coating on the dielectric layer) in the region/chamber 202, as also discussed above. Such frequency ranges and average or peak power ranges are known in the art. See, e.g., U.S. Pat. No. 6,958,132 (Chiou et al.), U.S. Pat. No. RE44,711 (Wu et al.) (originally issued as U.S. Pat. No. 7,612,355), and US Patent Application Publication Nos. US2014/0124370 (Short et al.), US2015/0306598 (Khandros et al.), and US2015/0306599 (Khandros et al.).

Sequestration pens. Non-limiting examples of generic sequestration pens 224, 226, and 228 are shown within the microfluidic device 230 depicted in FIGS. 2A-2C. Each sequestration pen 224, 226, and 228 can comprise an isolation structure 232 defining an isolation region 240 and a connection region 236 fluidically connecting the isolation region 240 to a channel 122. The connection region 236 can comprise a proximal opening 234 to the microfluidic channel 122 and a distal opening 238 to the isolation region 240. The connection region 236 can be configured so that the maximum penetration depth of a flow of a fluidic medium (not shown) flowing from the microfluidic channel 122 into the sequestration pen 224, 226, 228 does not extend into the isolation region 240. Thus, due to the connection region 236, a micro-object (not shown) or other material (not shown) disposed in an isolation region 240 of a sequestration pen 224, 226, 228 can thus be isolated from, and not substantially affected by, a flow of medium 180 in the microfluidic channel 122.

The sequestration pens 224, 226, and 228 of FIGS. 2A-2C each have a single opening which opens directly to the microfluidic channel 122. The opening of the sequestration pen opens laterally from the microfluidic channel 122. The electrode activation substrate 206 underlays both the microfluidic channel 122 and the sequestration pens 224, 226, and 228. The upper surface of the electrode activation substrate 206 within the enclosure of a sequestration pen, forming the floor of the sequestration pen, is disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 microns, 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be less than about 3%, 2%, 1%. 0.9%, 0.8%, 0.5%, 0.3% or 0.1% of the height of the walls of the sequestration pen or walls of the microfluidic device. While described in detail for the microfluidic device 200, this also applies to any of the microfluidic devices 100, 230, 250, 280, 290, 320, 400, 450, 500, 700 described herein.

The microfluidic channel 122 can thus be an example of a swept region, and the isolation regions 240 of the sequestration pens 224, 226, 228 can be examples of unswept regions. As noted, the microfluidic channel 122 and sequestration pens 224, 226, 228 can be configured to contain one or more fluidic media 180. In the example shown in FIGS. 2A-2B, the ports 222 are connected to the microfluidic channel 122 and allow a fluidic medium 180 to be introduced into or removed from the microfluidic device 230. Prior to introduction of the fluidic medium 180, the microfluidic device may be primed with a gas such as carbon dioxide gas. Once the microfluidic device 230 contains the fluidic medium 180, the flow 242 of fluidic medium 180 in the microfluidic channel 122 can be selectively generated and stopped. For example, as shown, the ports 222 can be disposed at different locations (e.g., opposite ends) of the microfluidic channel 122, and a flow 242 of medium can be created from one port 222 functioning as an inlet to another port 222 functioning as an outlet.

FIG. 2C illustrates a detailed view of an example of a sequestration pen 224 according to the present disclosure. Examples of micro-objects 246 are also shown.

As is known, a flow 242 of fluidic medium 180 in a microfluidic channel 122 past a proximal opening 234 of sequestration pen 224 can cause a secondary flow 244 of the medium 180 into and/or out of the sequestration pen 224. To isolate micro-objects 246 in the isolation region 240 of a sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ of the secondary flow 244 depends upon the velocity of the fluidic medium 180 flowing in the microfluidic channel 122 and various parameters relating to the configuration of the microfluidic channel 122 and the proximal opening 234 of the connection region 236 to the microfluidic channel 122. For a given microfluidic device, the configurations of the microfluidic channel 122 and the opening 234 will be fixed, whereas the rate of flow 242 of fluidic medium 180 in the microfluidic channel 122 will be variable. Accordingly, for each sequestration pen 224, a maximal velocity Vₘₐₓ for the flow 242 of fluidic medium 180 in channel 122 can be identified that ensures that the penetration depth Dₚ of the secondary flow 244 does not exceed the length L_{con} of the connection region 236. As long as the rate of the flow 242 of fluidic medium 180 in the microfluidic channel 122 does not exceed the maximum velocity Vₘₐₓ, the resulting secondary flow 244 can be limited to the microfluidic channel 122 and the connection region 236 and kept out of the isolation region 240. The flow 242 of medium 180 in the microfluidic channel 122 will thus not draw micro-objects 246 out of the isolation region 240. Rather, micro-objects 246 located in the isolation region 240 will stay in the isolation region 240 regardless of the flow 242 of fluidic medium 180 in the microfluidic channel 122.

Moreover, as long as the rate of flow 242 of medium 180 in the microfluidic channel 122 does not exceed Vₘₐₓ, the flow 242 of fluidic medium 180 in the microfluidic channel 122 will not move miscellaneous particles (e.g., microparticles and/or nanoparticles) from the microfluidic channel 122 into the isolation region 240 of a sequestration pen 224. Having the length L_{con} of the connection region 236 be greater than the maximum penetration depth Dₚ of the secondary flow 244 can thus prevent contamination of one sequestration pen 224 with miscellaneous particles from the microfluidic channel 122 or another sequestration pen (e.g., sequestration pens 226, 228 in FIG. 2D).

Because the microfluidic channel 122 and the connection regions 236 of the sequestration pens 224, 226, 228 can be affected by the flow 242 of medium 180 in the microfluidic channel 122, the microfluidic channel 122 and connection regions 236 can be deemed swept (or flow) regions of the microfluidic device 230. The isolation regions 240 of the sequestration pens 224, 226, 228, on the other hand, can be deemed unswept (or non-flow) regions. For example, components (not shown) in a first fluidic medium 180 in the microfluidic channel 122 can mix with a second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange. The first medium 180 can be the same medium or a different medium than the second medium 248. Moreover, the first medium 180 and the second medium 248 can start out being the same, then become different (e.g., through conditioning of the second medium 248 by one or more cells in the isolation region 240, or by changing the medium 180 flowing through the microfluidic channel 122).

The maximum penetration depth Dₚ of the secondary flow 244 caused by the flow 242 of fluidic medium 180 in the microfluidic channel 122 can depend on a number of parameters, as mentioned above. Examples of such parameters include: the shape of the microfluidic channel 122 (e.g., the microfluidic channel can direct medium into the connection region 236, divert medium away from the connection region 236, or direct medium in a direction substantially perpendicular to the proximal opening 234 of the connection region 236 to the microfluidic channel 122); a width W_{ch} (or cross-sectional area) of the microfluidic channel 122 at the proximal opening 234; and a width W_{con} (or cross-sectional area) of the connection region 236 at the proximal opening 234; the velocity V of the flow 242 of fluidic medium 180 in the microfluidic channel 122; the viscosity of the first medium 180 and/or the second medium 248, or the like.

In some embodiments, the dimensions of the microfluidic channel 122 and sequestration pens 224, 226, 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226, 228 can be in other orientations with respect to each other.

As illustrated in FIG. 2C, the width con of W_{con} the connection region 236 can be uniform from the proximal opening 234 to the distal opening 238. The width W_{con} of the connection region 236 at the distal opening 238 can thus be any of the values identified herein for the width W_{con} of the connection region 236 at the proximal opening 234. Alternatively, the width W_{con} of the connection region 236 at the distal opening 238 can be larger than the width W_{con} of the connection region 236 at the proximal opening 234.

As illustrated in FIG. 2C, the width of the isolation region 240 at the distal opening 238 can be substantially the same as the width W_{con} of the connection region 236 at the proximal opening 234. The width of the isolation region 240 at the distal opening 238 can thus be any of the values identified herein for the width W_{con} of the connection region 236 at the proximal opening 234. Alternatively, the width of the isolation region 240 at the distal opening 238 can be larger or smaller than the width W_{con} of the connection region 236 at the proximal opening 234. Moreover, the distal opening 238 may be smaller than the proximal opening 234 and the width W_{con} of the connection region 236 may be narrowed between the proximal opening 234 and distal opening 238. For example, the connection region 236 may be narrowed between the proximal opening and the distal opening, using a variety of different geometries (e.g. chamfering the connection region, beveling the connection region). Further, any part or subpart of the connection region 236 may be narrowed (e.g. a portion of the connection region adjacent to the proximal opening 234).

FIGS. 2D-2F depict another exemplary embodiment of a microfluidic device 250 containing a microfluidic circuit 262 and flow channels 264, which are variations of the respective microfluidic device 100, circuit 132 and channel 134 of FIG. 1A. The microfluidic device 250 also has a plurality of sequestration pens 266 that are additional variations of the above-described sequestration pens 124, 126, 128, 130, 224, 226 or 228. In particular, it should be appreciated that the sequestration pens 266 of device 250 shown in FIGS. 2D-2F can replace any of the above-described sequestration pens 124, 126, 128, 130, 224, 226 or 228 in devices 100, 200, 230, 280, 290, 300. Likewise, the microfluidic device 250 is another variant of the microfluidic device 100, and may also have the same or a different DEP configuration as the above-described microfluidic device 100, 200, 230, 280, 290, 300, as well as any of the other microfluidic system components described herein.

The microfluidic device 250 of FIGS. 2D-2F comprises a support structure (not visible in FIGS. 2D-2F, but can be the same or generally similar to the support structure 104 of device 100 depicted in FIG. 1A), a microfluidic circuit structure 256, and a cover (not visible in FIGS. 2D-2F, but can be the same or generally similar to the cover 122 of device 100 depicted in FIG. 1A). The microfluidic circuit structure 256 includes a frame 252 and microfluidic circuit material 260, which can be the same as or generally similar to the frame 114 and microfluidic circuit material 116 of device 100 shown in FIG. 1A. As shown in FIG. 2D, the microfluidic circuit 262 defined by the microfluidic circuit material 260 can comprise multiple channels 264 (two are shown but there can be more) to which multiple sequestration pens 266 are fluidically connected.

Each sequestration pen 266 can comprise an isolation structure 272, an isolation region 270 within the isolation structure 272, and a connection region 268. From a proximal opening 274 at the microfluidic channel 264 to a distal opening 276 at the isolation structure 272, the connection region 268 fluidically connects the microfluidic channel 264 to the isolation region 270. Generally, in accordance with the above discussion of FIGS. 2B and 2C, a flow 278 of a first fluidic medium 254 in a channel 264 can create secondary flows 282 of the first medium 254 from the microfluidic channel 264 into and/or out of the respective connection regions 268 of the sequestration pens 266.

As illustrated in FIG. 2E, the connection region 268 of each sequestration pen 266 generally includes the area extending between the proximal opening 274 to a channel 264 and the distal opening 276 to an isolation structure 272. The length L_{con} of the connection region 268 can be greater than the maximum penetration depth Dₚ of secondary flow 282, in which case the secondary flow 282 will extend into the connection region 268 without being redirected toward the isolation region 270 (as shown in FIG. 2D). Alternatively, at illustrated in FIG. 2F, the connection region 268 can have a length L_{con} that is less than the maximum penetration depth Dₚ, in which case the secondary flow 282 will extend through the connection region 268 and be redirected toward the isolation region 270. In this latter situation, the sum of lengths L_{c1} and L_{c2} of connection region 268 is greater than the maximum penetration depth Dₚ, so that secondary flow 282 will not extend into isolation region 270. Whether length L_{con} of connection region 268 is greater than the penetration depth Dₚ, or the sum of lengths L_{c1} and L_{c2} of connection region 268 is greater than the penetration depth Dₚ, a flow 278 of a first medium 254 in channel 264 that does not exceed a maximum velocity Vₘₐₓ will produce a secondary flow having a penetration depth Dₚ, and micro-objects (not shown but can be the same or generally similar to the micro-objects 246 shown in FIG. 2C) in the isolation region 270 of a sequestration pen 266 will not be drawn out of the isolation region 270 by a flow 278 of first medium 254 in channel 264. Nor will the flow 278 in channel 264 draw miscellaneous materials (not shown) from channel 264 into the isolation region 270 of a sequestration pen 266. As such, diffusion is the only mechanism by which components in a first medium 254 in the microfluidic channel 264 can move from the microfluidic channel 264 into a second medium 258 in an isolation region 270 of a sequestration pen 266. Likewise, diffusion is the only mechanism by which components in a second medium 258 in an isolation region 270 of a sequestration pen 266 can move from the isolation region 270 to a first medium 254 in the microfluidic channel 264. The first medium 254 can be the same medium as the second medium 258, or the first medium 254 can be a different medium than the second medium 258. Alternatively, the first medium 254 and the second medium 258 can start out being the same, then become different, e.g., through conditioning of the second medium by one or more cells in the isolation region 270, or by changing the medium flowing through the microfluidic channel 264.

As illustrated in FIG. 2E, the width W_{ch} of the microfluidic channels 264 (i.e., taken transverse to the direction of a fluid medium flow through the microfluidic channel indicated by arrows 278 in FIG. 2D) in the microfluidic channel 264 can be substantially perpendicular to a width W_{con1} of the proximal opening 274 and thus substantially parallel to a width W_{con2} of the distal opening 276. The width W_{con1} of the proximal opening 274 and the width W_{con2} of the distal opening 276, however, need not be substantially perpendicular to each other. For example, an angle between an axis (not shown) on which the width W_{con1} of the proximal opening 274 is oriented and another axis on which the width W_{con2} of the distal opening 276 is oriented can be other than perpendicular and thus other than 90°. Examples of alternatively oriented angles include angles of: about 30° to about 90°, about 45° to about 90°, about 60° to about 90°, or the like.

In various embodiments of sequestration pens (e.g. 124, 126, 128, 130, 224, 226, 228, or 266), the isolation region (e.g. 240 or 270) is configured to contain a plurality of micro-objects. In other embodiments, the isolation region can be configured to contain only one, two, three, four, five, or a similar relatively small number of micro-objects. Accordingly, the volume of an isolation region can be, for example, at least 1×10⁶, 2×10⁶, 4×10⁶, 6×10⁶ cubic microns, or more.

In various embodiments of sequestration pens, the width W_{ch} of the microfluidic channel (e.g., 122) at a proximal opening (e.g. 234) can be about 50-1000 microns, 50-500 microns, 50-400 microns, 50-300 microns, 50-250 microns, 50-200 microns, 50-150 microns, 50-100 microns, 70-500 microns, 70-400 microns, 70-300 microns, 70-250 microns, 70-200 microns, 70-150 microns, 90-400 microns, 90-300 microns, 90-250 microns, 90-200 microns, 90-150 microns, 100-300 microns, 100-250 microns, 100-200 microns, 100-150 microns, or 100-120 microns. In some other embodiments, the width W_{ch} of the microfluidic channel (e.g., 122) at a proximal opening (e.g. 234) can be about 200-800 microns, 200-700 microns, or 200-600 microns. The foregoing are examples only, and the width W_{ch} of the microfluidic channel 122 can be any width within any of the endpoints listed above. Moreover, the W_{ch} of the microfluidic channel 122 can be selected to be in any of these widths in regions of the microfluidic channel other than at a proximal opening of a sequestration pen. In some embodiments, a sequestration pen has a height of about 30 to about 200 microns, or about 50 to about 150 microns. In some embodiments, the sequestration pen has a cross-sectional area of about 1×10⁴-3×10⁶ square microns, 2×10⁴-2×10⁶ square microns, 4×10⁴-1×10⁶ square microns, 2×10⁴-5×10⁵ square microns, 2×10⁴-1×10⁵ square microns or about 2×10⁵-2×10⁶ square microns.

In various embodiments of sequestration pens, the height H_{ch} of the microfluidic channel (e.g., 122) at a proximal opening (e.g., 234) can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{ch} of the microfluidic channel (e.g., 122) can be a height within any of the endpoints listed above. The height H_{ch} of the microfluidic channel 122 can be selected to be in any of these heights in regions of the microfluidic channel other than at a proximal opening of a sequestration pen.

In various embodiments of sequestration pens a cross-sectional area of the microfluidic channel (e.g., 122) at a proximal opening (e.g., 234) can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel (e.g., 122) at a proximal opening (e.g., 234) can be any area within any of the endpoints listed above.

In various embodiments of sequestration pens, the length L_{con} of the connection region (e.g., 236) can be about 1-600 microns, 5-550 microns, 10-500 microns, 15-400 microns, 20-300 microns, 20-500 microns, 40-400 microns, 60-300 microns, 80-200 microns, or about 100-150 microns. The foregoing are examples only, and length L_{con} of a connection region (e.g., 236) can be in any length within any of the endpoints listed above.

In various embodiments of sequestration pens the width W_{con} of a connection region (e.g., 236) at a proximal opening (e.g., 234) can be about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-250 microns, 50-200 microns, 50-150 microns, 50-100 microns, 50-80 microns, 60-200 microns, 60-150 microns, 60-100 microns, 60-80 microns, 70-150 microns, 70-100 microns, or 80-100 microns. The foregoing are examples only, and the width W_{con} of a connection region (e.g., 236) at a proximal opening (e.g., 234) can be different than the foregoing examples (e.g., any value within any of the endpoints listed above).

In various embodiments of sequestration pens, the width W_{con} of a connection region (e.g., 236) at a proximal opening (e.g., 234) can be at least as large as the largest dimension of a micro-object (e.g., biological cell, which may be a B cell, a plasma cell, a hybridoma, a recombinant antibody secreting cell (ASC), such as a CHO cell or a yeast cell, or the like) that the sequestration pen is intended for. The foregoing are examples only, and the width W_{con} of a connection region (e.g., 236) at a proximal opening (e.g., 234) can be different than the foregoing examples (e.g., a width within any of the endpoints listed above).

In various embodiments of sequestration pens, the width Wₚᵣ of a proximal opening of a connection region may be at least as large as the largest dimension of a micro-object (e.g., a biological micro-object such as a cell) that the sequestration pen is intended for. For example, the width Wₚᵣ may be about 50 microns, about 60 microns, about 100 microns, about 200 microns, about 300 microns or may be about 50-300 microns, about 50-200 microns, about 50-100 microns, about 75-150 microns, about 75-100 microns, or about 200-300 microns.

In various embodiments of sequestration pens, a ratio of the length L_{con} of a connection region (e.g., 236) to a width W_{con} of the connection region (e.g., 236) at the proximal opening 234 can be greater than or equal to any of the following ratios: 0.5, 1.0, 1.5, 2.0, 2.5, 3.0, 3.5, 4.0, 4.5, 5.0, 6.0, 7.0, 8.0, 9.0, 10.0, or more. The foregoing are examples only, and the ratio of the length L_{con} of a connection region 236 to a width W_{con} of the connection region 236 at the proximal opening 234 can be different than the foregoing examples.

In various embodiments of microfluidic devices 100, 200, 23, 250, 280, 290, 300, Vₘₐₓ can be set around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, or 20 microliters/sec.

In various embodiments of microfluidic devices having sequestration pens, the volume of an isolation region (e.g., 240) of a sequestration pen can be, for example, at least 5×10⁵, 8×10⁵, 1×10⁶, 2×10⁶, 4×10⁶, 6×10⁶, 8×10⁶, 1×10⁷, 5×10⁷, 1×10⁸, 5×10⁸, or 8×10⁸ cubic microns, or more. In various embodiments of microfluidic devices having sequestration pens, the volume of a sequestration pen may be about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some other embodiments, the volume of a sequestration pen may be about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters.

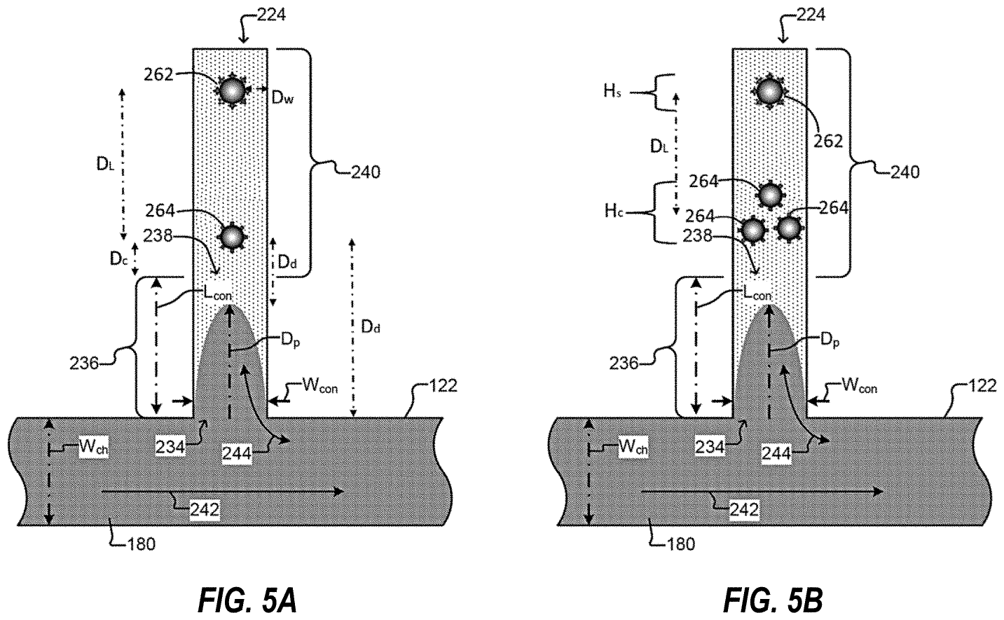

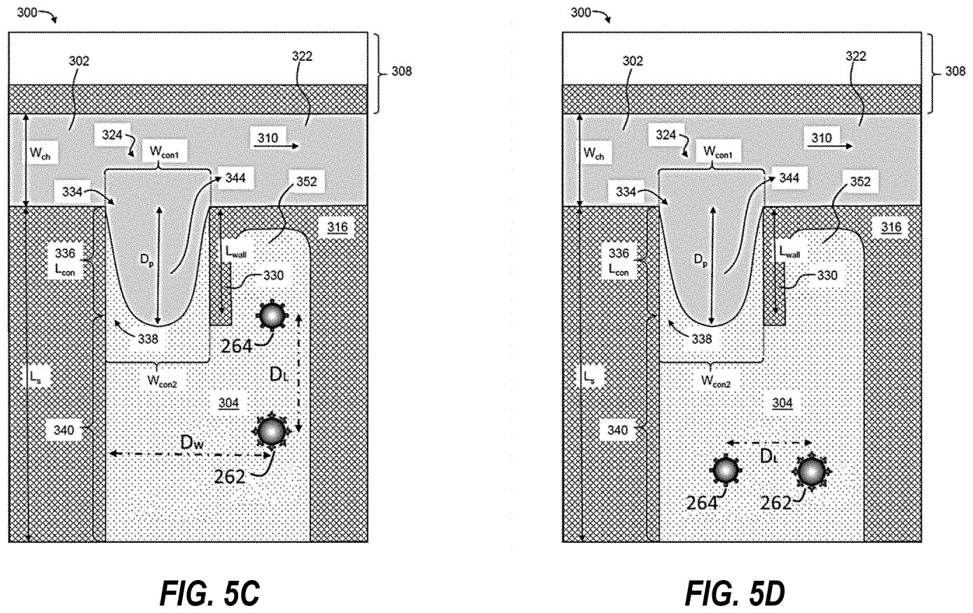

In some embodiments, an isolation region of a sequestration pen has a length (determined as Lₛ−L_{con}, referring to FIG. 5C) of about 40-600 microns, about 40-500 microns, about 40-400 microns, about 40-300 microns, about 40-200 microns, about 40-100 microns or about 40-80 microns. In some embodiments, an isolation region of a sequestration pen has a length of about 30-550 microns, about 30-450 microns, about 30-350 microns, about 30-250 microns, about 30-170 microns, about 30-80 microns or about 30-70 microns. The foregoing are examples only, and a sequestration pen may have a length Lₛ selected to be between any of the values listed above.

In various embodiment, the microfluidic device has sequestration pens configured as in any of the embodiments discussed herein where the microfluidic device has about 5 to about 10 sequestration pens, about 10 to about 50 sequestration pens, about 100 to about 500 sequestration pens; about 200 to about 1000 sequestration pens, about 500 to about 1500 sequestration pens, about 1000 to about 2000 sequestration pens, about 1000 to about 3500 sequestration pens, about 3000 to about 7000 sequestration pens, about 5000 to about 10,000 sequestration pens, about 9,000 to about 15,000 sequestration pens, or about 12,000 to about 20,000 sequestration pens. The sequestration pens need not all be the same size and may include a variety of configurations (e.g., different widths, different features within the sequestration pen).

FIG. 2G illustrates a microfluidic device 280 according to one embodiment. The microfluidic device 280 illustrated in FIG. 2G is a stylized diagram of a microfluidic device 100. In practice the microfluidic device 280 and its constituent circuit elements (e.g. channels 122 and sequestration pens 128) would have the dimensions discussed herein. The microfluidic circuit 120 illustrated in FIG. 2G has two ports 107, four distinct channels 122 and four distinct flow paths 106. The microfluidic device 280 further comprises a plurality of sequestration pens opening off of each channel 122. In the microfluidic device illustrated in FIG. 2G, the sequestration pens have a geometry similar to the pens illustrated in FIG. 2C and thus, have both connection regions and isolation regions. Accordingly, the microfluidic circuit 120 includes both swept regions (e.g. channels 122 and portions of the connection regions 236 within the maximum penetration depth Dₚ of the secondary flow 244) and non-swept regions (e.g. isolation regions 240 and portions of the connection regions 236 not within the maximum penetration depth Dₚ of the secondary flow 244).

FIG. 2G depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 2G includes a microfluidic channel 322, having a width W_{ch}, as described herein, and containing a flow 310 of first fluidic medium 302 and one or more sequestration pens 324 (only one illustrated in FIG. 2G). The sequestration pens 324 each have a length Lₛ, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{con} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352. In sequestration pens configured like those of FIGS. 2A-2G, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion. Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Sequestration pen dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure, as follows.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25. times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Lₛ of about 40-600 microns, about 40-500 microns, about 40-400 microns, about 40-300 microns, about 40-200 microns, about 40-100 microns or about 40-80 microns. The foregoing are examples only, and a sequestration pen may have a length Lₛ selected to be between any of the values listed above.

According to some embodiments, a sequestration pen may have a specified height (e.g., Hₛ). In some embodiments, a sequestration pen has a height Hₛ of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hₛ selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hₛ of the sequestration pen is typically selected to be the same as the height H_{con} of a connection region and/or the height H_{ch} of the microfluidic channel. In some embodiments, Hₛ, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, or 20 microliters/sec. The foregoing are examples only, and the flow of fluidic medium within the microfluidic channel can have a maximum velocity (e.g., Vₘₐₓ) selected to be a value between any of the values listed above.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

### IV. Methods for Assaying a Binding Affinity in a Microfluidic Device

Methods for assaying a binding affinity between a first molecule and a second molecule in a micro-fluidic device described above are provided. As described above, the micro-fluidic device comprises a flow region and a chamber that opens off of the flow region.

In some embodiments, the method comprises: providing the second molecule into the chamber, wherein the second molecule is labeled with a signal-emitting moiety and a first capture micro-object comprising the first molecule is present in the chamber, and allowing the second molecule to bind to the first molecule of the first capture micro-object, wherein the binding of the second molecule to the first molecule is allowed to proceed to saturation; removing unbound second molecule from the microfluidic device; providing a second capture micro-object into the chamber, wherein the second capture micro-object comprises a third molecule which specifically binds to the second molecule; detecting over a period of time a decrease in the amount of second molecule bound to the first capture micro-object; optionally detecting over the period of time an increase in amount of second molecule bound to the second capture micro-object; and determining the relative binding affinity between the first molecule and the second molecule.

In some embodiments, the binding affinity between the first molecule and the second molecule is determined based on the decrease in amount of second molecule bound to the first capture micro-object over the period of time. In some embodiments, the binding affinity between the first molecule and the second molecule is calculated based on a ratio of (i) the increase in the amount of second molecule bound to the second capture micro-object over the period of time to (ii) the decrease in amount of second molecule bound to the first capture micro-object over the period of time.

In some embodiments, the method comprises: providing a second molecule labeled with a signal-emitting moiety into the chamber, wherein a first capture micro-object comprising the first molecule is present in the chamber, and allowing the second molecule to bind to the first molecule of the first capture micro-object, wherein the binding of the second molecule to the first molecule is allowed to proceed to saturation; depleting unbound second molecule from the microfluidic device; detecting over a period of time a decrease in amount of the second molecule bound to the first capture micro-object; and determining the relative binding affinity between the first molecule and the second molecule based on the decrease in amount of the second molecule bound to the first capture micro-object over the period of time.

In some embodiments, determining the relative binding affinity between the first molecule and the second molecule comprises calculating a dissociation rate constant (k_{off}) for the first and second molecules. In some embodiments, the k_{off} is determined to be in the range of about 1.0×10⁻⁵ to about 1.0×10⁻³ s⁻¹. In some embodiments, the k_{off} is determined to be in the range of about 1×10⁻⁶ to about 1×10⁻³ s⁻¹, about 5×10⁻⁶ to about 1×10⁻³ s⁻¹, about 1×10⁻⁵ to about 1×10⁻³ s⁻¹, about 5×10⁻⁵ to about 1×10⁻³ s⁻¹, about 1×10⁻⁴ to about 1×10⁻³ s⁻¹, about 1×10⁻⁶ to about 5×10's⁻¹, about 1×10⁻⁶ to about 1×10's⁻¹, about 1×10⁻⁶ to about 5×10⁻⁵ s⁻¹, about 5×10⁻⁵ to about 1×10⁻³ s⁻¹.

In some embodiments, determining the relative binding affinity between the first molecule and the second molecule comprises dividing the dissociation rate constant (k_{off}) for the first and second molecules by an association rate constant (kₒₙ). In some embodiments, kₒₙ is an estimated value (e.g., estimated based on known association rate constants for molecules similar to the first and second molecules). In some embodiments, a kₒₙ value in the range of about 1×10⁶ to about 1×10⁷ M⁻¹s⁻¹ is used.

In some embodiments, providing the second molecule into the chamber comprises:
flowing a solution comprising the second molecule through the flow path in the microfluidic device; and allowing the second molecule to diffuse into the chamber.

In some embodiments, the method further comprises, prior to the step of providing the second molecule into the chamber, providing the first capture micro-object into the chamber.

In some embodiments, the chamber is a first chamber and the method comprises, prior to providing the first capture micro-object into the chamber, disposing a first capture micro-object into a second chamber in which the first molecule is present, and allowing the first molecule to bind to the first capture micro-object in the second chamber, optionally wherein the second chamber is adjacent to the first chamber. The method may then continue as discussed above, e.g., with providing the first capture micro-object into the first chamber, providing the second molecule, etc. In some embodiments, after removal of the unbound second molecule from the microfluidic device and before providing the second capture micro-object is then provided into the first chamber, the method comprises providing the second capture micro-object into the second chamber in which the first molecule is present, and allowing the first molecule to bind to the second capture micro-object in the second chamber.

In some embodiments, the method further comprises, prior to and/or simultaneously with allowing the first molecule to bind to the first capture micro-object in the second chamber and/or allowing the first molecule to bind to the second capture micro-object in the second chamber: culturing one or more biological cells in the second chamber, wherein the one or more biological cells secrete the first molecule.

Capture Micro-Objects.

In some embodiments, the first and/or second capture micro-object comprises a microparticle, a microbead, or a magnetic bead. In some embodiments, first and/or second micro-object comprises microbeads (e.g., polymer beads, glass beads, polystyrene beads, Luminex™ beads, or any other beads commercially available, or the like). The first and/or second capture micro-object may comprise a first molecule, covalently or non-covalently attached, such as fluorescent labels, nucleic acids (e.g., oligonucleotides), proteins, antibodies, carbohydrates, antigens, small molecule signaling moieties, or other chemical/biological species capable of use in an assay. In some embodiments, the first and/or second capture micro-object is a microbead comprising a first molecule. In some embodiments, the first and/or second capture micro-object has a largest dimension from 1 μm to 50 μm, from 5 μm to 40 μm, from 10 μm to 30 μm, or from 10 μm to 25 μm. In some embodiments, the first and/or second capture micro-object has a largest dimension from 10 μm to 25 μm. In some embodiments, the first and second capture micro-objects have the same largest dimension. In some embodiments, the first and second capture micro-objects have different largest dimensions.

First molecule and Second molecule. In some embodiments, the first molecule is an antibody or an antigen-binding fragment thereof. In some embodiments, the second molecule is an antigen. In some embodiments, the antigen is an antigen expressed by a pathogenic agent (e.g., a virus, a bacterium, a cancer cell, or the like). In some embodiments, the antigen is a peptide, an extracellular signaling molecule, or a cell-surface protein. In some embodiments, the signal-emitting moiety comprises a fluorophore.

Positions of Capture Micro-Objects

As described above, the microfluidic device provided for the methods described herein comprises a flow region and a chamber that opens off of the flow region. Positions of first and/or second capture micro-objects may be provided as a distance from another capture micro-object in the same chamber, a distance from a structural component of the chamber (or pen).

In some embodiments, the microfluidic device comprises a housing, and the housing comprises a base and a microfluidic structure disposed on the base. In some embodiments, the flow path comprises a microfluidic channel, and wherein the chamber opens off of the microfluidic channel. In some embodiments, the chamber is micro-well formed in the base of the housing. In some embodiments, the chamber is a sequestration pen. Examples of chambers used herein have been described, for example, in U.S. Pat. Application No. 2012/0009671, the contents of which are incorporated herein by reference.

In some embodiments, each sequestration pen comprises an isolation region having a single opening, and a connection region, the connection region having a proximal opening to the flow region (or channel) and a distal opening to the isolation region. The isolation region can be an unswept region of the microfluidic device.

In some embodiments, the connection region comprises a proximal opening into the flow region (or microfluidic channel) having a width W_{con} ranging from about 20 microns to about 100 microns and a distal opening into said isolation region, and wherein a length L_{con} of said connection region from the proximal opening to the distal opening is as least 1.0 times a width W_{con} of the proximal opening of the connection region. In some embodiments, the length L_{con} of the connection region from the proximal opening to the distal opening is at least 1.5 times the width W_{con} of the proximal opening of the connection region.

In some embodiments, the length L_{con} of the connection region from the proximal opening to the distal opening is at least 2.0 times the width con of W_{con} the proximal opening of the connection region. In some embodiments, the width W_{con} of the proximal opening of the connection region ranges from about 20 microns to about 60 microns. In some embodiments, the length L_{con} of the connection region from the proximal opening to the distal opening is between about 20 microns and about 500 microns. In some embodiments, a width of the microfluidic channel at the proximal opening of the connection region is between about 50 microns and about 500 microns. In some embodiments, a height of the microfluidic channel at the proximal opening of the connection region is between 20 microns and 100 microns. In some embodiments, the proximal opening of the connection region is parallel to a direction of the flow of a first medium in the flow region.

In some embodiments, the width of the isolation region at the distal opening is substantially the same as the width of the connection region at the proximal opening, and larger than the largest dimension of the first and second capture micro-objects. In some embodiments, during the detecting step, the first capture micro-object and the second capture micro-object are present in the isolation region of the chamber. In some embodiments, during the detecting step, the distance between the first capture micro-object and the second capture micro-object (D_{L}) is equal to or smaller than the entire length of the isolation region. In some embodiments, D_{L} is in a range from a first fraction to a second fraction of the length of the isolation region, wherein the first and second fraction are respectively 0.1 and 0.2; 0.2 and 0.3; 0.3 and 0.4; 0.4 and 0.5; 0.5 and 0.6; 0.6 and 0.7; 0.7 and 0.8; 0.8 and 0.9; or 0.9 and 1. In some embodiments, the distance of the second capture micro-object from the proximal opening of the connection region (D_{d}) is smaller than the distance of the first capture micro-object from the proximal opening of the connection region (D_{d}+D_{L}). In some embodiments, the D_{L} is about 20 microns to about 200 microns, 20 microns to 180 microns, 20 microns to 160 microns, 20 microns to 140 microns, 20 microns to 120 microns, 20 microns to 100 microns, 20 microns to 90 microns, 30 microns to 200 microns, 30 microns to 180 microns, 30 microns to 160 microns, 30 microns to 140 microns, 30 microns to 120 microns, 30 microns to 100 microns, 30 microns to 90 microns, 40 microns to 200 microns, 40 microns to 180 microns, 40 microns to 160 microns, 40 microns to 140 microns, 40 microns to 120 microns, 40 microns to 100 microns, 40 microns to 90 microns, 40 microns to 60 microns, 50 microns to 200 microns, 50 microns to 180 microns, 50 microns to 160 microns, 50 microns to 140 microns, 50 microns to 120 microns, 50 microns to 100 microns, 50 microns to 90 microns, 60 microns to 200 microns, 60 microns to 180 microns, 60 microns to 160 microns, 60 microns to 140 microns, 60 microns to 120 microns, 60 microns to 100 microns, 60 microns to 90 microns, 80 microns to 200 microns, 80 microns to 180 microns, 80 microns to 160 microns, 80 microns to 140 microns, 80 microns to 120 microns, 80 microns to 100 microns, 80 microns to 90 microns, or about 90 microns. In some embodiments, the second capture micro-object is positioned away from the connection region by a distance, D_{c}. In some embodiments, D_{c} is equal to or larger than equal to or larger than 10 microns (e.g., at least 15 microns, 20 microns, 25 microns, 30 microns, 35 microns, 40 microns, 45 microns, 50 microns, or more). In some embodiments, the distance of the second capture micro-object from the proximal opening of the connection region (D_{d}) is longer than the penetration depth (Dₚ) of the first fluidic medium flowing from the flowing region.

FIG. 5A illustrates a detailed view of an example of a sequestration pen 224 according to the present disclosure. The sequestration pen 224 is essentially the same as the sequestration pen 224 depicted in FIG. 2C and described above in Section III. Examples of first and second capture micro-objects 262, 264 are shown in FIG. 5A.

In FIG. 5A, the width of the isolation region at the distal opening is substantially the same as the width of the connection region W_{con} at the proximal opening, and larger than the largest dimension of the first and second capture micro-objects 262, 264. The first capture micro-object 262 and the second capture micro-object 264 are present in the isolation region 240 of the chamber 224. The distance between the first capture micro-object and the second capture micro-object (D_{L}) is equal to or smaller than the entire length of the isolation region 240. The distance of the second capture micro-object 264 from the proximal opening of the connection region (D_{d}) is smaller than the distance of the first capture micro-object from the proximal opening of the connection region (D_{d}+D_{L}). The second capture micro-object 264 is present away from the connection region 236 by a distance, D_{c}. The distance of the second capture micro-object 264 from the proximal opening of the connection region (D_{d}) is longer than the penetration depth (Dₚ) of the first fluidic medium flowing from the flowing region.

Multiple First and/or Second Capture Micro-Objects

In some embodiments, the first capture micro-object comprises a plurality of first capture micro-objects, each comprising the first molecule. In some embodiments, the method described herein further comprises allowing the second molecule to bind to the first molecule of each of the plurality of first capture micro-objects, wherein the binding of the second molecule to the first molecule is allowed to proceed to saturation. In some embodiments, the method described herein further comprises detecting over a period of time a decrease in amount of second molecule bound to the plurality of first capture micro-objects.

In some embodiments, the method described herein further comprises: determining the relative binding affinity between the first molecule and the second molecule based on a ratio of (i) the increase in the amount of second molecule bound to the second capture micro-object over the period of time to (ii) the decrease in amount of second molecule bound to each of the plurality of first capture micro-objects over the period of time. In some embodiments, the method described herein further comprises: determining the relative binding affinity between the first molecule and the second molecule based on a ratio of (i) the increase in the amount of second molecule bound to the second capture micro-object over the period of time to (ii) the total decrease in amount of second molecule bound to the plurality of first capture micro-objects over the period of time.

In some embodiments, the second capture micro-object comprises a plurality of second capture micro-objects, each comprising the first molecule. In some embodiments, the method described herein further comprises detecting over a period of time an increase in amount of second molecule bound to the plurality of second capture micro-objects. In some embodiments, the method described herein further comprises determining the relative binding affinity between the first molecule and the second molecule based on a ratio of (i) the total increase in the amount of second molecule bound to the plurality of second capture micro-objects over the period of time to (ii) the decrease in amount of second molecule bound to the first capture micro-object over the period of time.

In some embodiments, during the detecting step, the first capture micro-object and the plurality of second capture micro-objects are present in the isolation region of the chamber. In some embodiments, the plurality of second capture micro-objects are proximal to the proximal opening of the connection region and the first capture micro-object is distal from the proximal opening of the connection region. In some embodiments, the plurality of second capture micro-objects include a most proximal second capture micro-object and a most distal second capture micro-object, defining a distance therebetween, H_{c}. In some embodiments, the sum of the distance H_{c} and the distance between the most proximal first capture micro-object and the first capture micro-object (D_{L}) is smaller than the entire length of the isolation region.

In some embodiments, during the detecting step, the plurality of first capture micro-objects and the second capture micro-object are present in the isolation region of the chamber. In some embodiments, the second capture micro-object from the proximal opening of the connection region is proximal to the proximal opening of the connection region and the plurality of first capture micro-objects are distal from the proximal opening of the connection region. In some embodiments, the plurality of first capture micro-objects include a most proximal first capture micro-object and a most distal first capture micro-object, defining a distance therebetween, H_{c}. In some embodiments, the sum of the distance H_{c} and the distance between the most proximal capture micro-object and the second capture micro-object (D_{L}) is smaller than the entire length of the isolation region.

In some embodiments, the H_{c} is about 5 microns to about 50 microns, about 10 microns to about 45 microns, about 10 microns to about 40 microns, about 15 microns to about 35 microns, about 20 microns to about 30 microns, about 10 microns, about 15 microns, about 20 microns, about 30 microns, about 35 microns, about 40 microns, about 45 microns, or about 50 microns.

In some embodiments, the proximal opening of the connection region is parallel to the direction of the flow of the first medium, and the distal opening of the isolation region is not parallel to the direction of the flow of the first medium. In some embodiments, the width W_{con2} of the distal opening of the connection region is substantially the same as the width W_{con1} of the proximal opening of the connection region, and is larger than the largest dimension of the first and second capture micro-objects. In some embodiments, the width W_{con2} of the distal opening of the connection region is larger or smaller as the width W_{con1} of the proximal opening of the connection region, and is larger than the largest dimension of the first and second capture micro-objects.

In some embodiments, during the detecting step, the first capture micro-object and the second capture micro-object are present in the isolation region of the sequestration pen. In some embodiments, the distance between the first capture micro-object and the second capture micro-object in a direction parallel to the length of the connection region, D_{L}, is equal to or smaller than the entire length of the isolation region. In some embodiments, the distance between the first capture micro-object and the second capture micro-object in a direction parallel to the width of the proximal opening of the connection region, D_{L}, is equal to or smaller than the width between opposite walls of the isolation region.

In some embodiments, the sequestration pen comprises a connection region wall laterally positioned with respect to the proximal opening and at least partially extends into the enclosed portion of the sequestration pen with the length L_{wall}, defining a hook region in the isolation region. In some embodiments, the second capture micro-object is present in or proximal to the hook region, and the first capture micro-object is distal from the hook region.

FIG. 5B illustrates a detailed view of an example of a sequestration pen 224 according to the present disclosure. The sequestration pen 224 is essentially the same as the sequestration pen 224 depicted in FIG. 2C and described above in Section III. Examples of a first capture micro-object 262 and a plurality of second capture micro-objects 264 are shown in FIG. 5B.

In FIG. 5B, the first capture micro-object 262 and the plurality of second capture micro-objects 264 are present in the isolation region 240 of the chamber 224. The second capture micro-objects 264 is proximal to the proximal opening of the connection region 236 and the first capture micro-object 262 is distal from the proximal opening of the connection region 236. The plurality of second capture micro-objects include a most proximal second capture micro-object and a most distal second capture micro-object, defining a distance therebetween, Hₛ. The sum of the distance Hₛ and the distance between the most proximal capture micro-object and the second capture micro-object is smaller than the entire length of the isolation region 240.

FIGS. 5C-5D illustrates a detailed view of examples of a sequestration pen 304 according to the present disclosure. The sequestration pen 304 is essentially the same as the sequestration pen 304 depicted in FIG. 2G and described above in Section III. Examples of a first capture micro-object 262 and a second capture micro-object 264 are shown in FIGS. 5C-5D.

In FIGS. 5C-5D, the proximal opening of the connection region 336 is parallel to the direction of the flow of the first medium, and the distal opening of the isolation region 336 is not parallel to the direction of the flow of the first medium. In the connection region 336, the width W_{con2} of the distal opening is substantially the same as the width W_{con1} of the proximal opening, and is larger than the largest dimension of the first and second capture micro-objects 262, 264. In the connection region 336, the width W_{con2} of the distal opening is larger or smaller as the width W_{con1} of the proximal opening, and is larger than the largest dimension of the first and second capture micro-objects 262, 264. The first capture micro-object 262 and the second capture micro-object 264 are present in the isolation region 340 of the sequestration pen 304. The sequestration pen 304 comprises a connection region wall 330 laterally positioned with respect to the proximal opening and at least partially extends into the enclosed portion of the sequestration pen with the length L_{wall}, defining a hook region 352 in the isolation region 340.

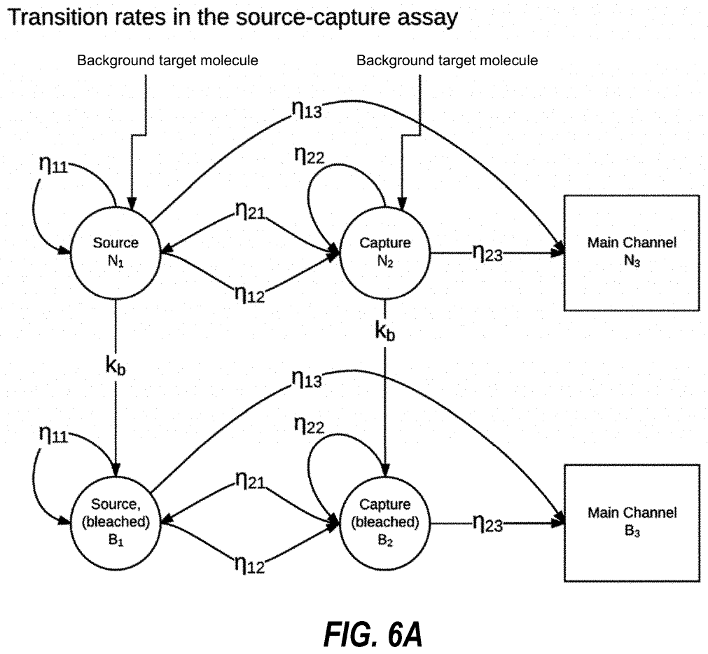

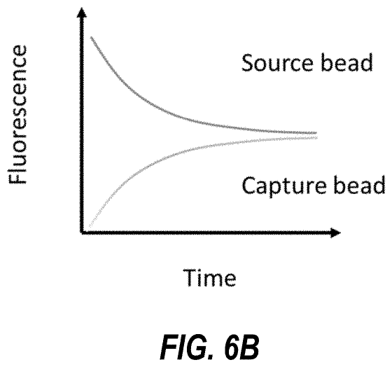

In FIG. 5C, the distance between the first capture micro-object 262 and the second capture micro-object 264 in a direction parallel to the length of the connection region, D_{L}, is equal to or smaller than the entire length of the isolation region 340. In FIG. 6C, the second capture micro-object 264 is present in or proximal to the hook region 352, and the first capture micro-object is distal from the hook region 352.

In FIG. 5D, the distance between the first capture micro-object 262 and the second capture micro-object 264 in a direction parallel to the width W_{con1} of the proximal opening of the connection region 261, D_{L}, is equal to or smaller than the width between opposite walls of the isolation region 261.

1) Determining Relative Binding Affinity

Theoretical modeling in the source-capture system described detailed below can be used to determine the binding affinity between a first molecule and a second molecule with the characteristic dissociation rate constant (k_{off}). The first capture micro-object and the second capture micro-object in the methods described herein correspond to a source bead and a capture bead in the source-capture system, respectively.

The source-capture system can be fully described by a set of probabilities describing the likelihood that a given dissociated antigen molecule will transition to a given location. FIG. 6A shows all the possible transitions a target molecule (e.g., antigen) can undergo.

As shown in FIG. 6A, six total states exist in this model:

N₁: number of unbleached molecules on the source bead

N₂: number of unbleached molecules on the capture bead

B₁: number of bleached molecules on the source bead

B₂: number of bleached molecules on the capture bead

N₃: number of unbleached molecules leaked to the main channel (not tracked)

B₃: number of bleached molecules leaked to the main channel (not tracked)

However, the last two states N₃, B₃ that account for leakage to the main channel can be inferred, and it is only necessary to account for the first four of them N₁, N₂, B₁, B₂. If a molecule sticks to the surface, it can be considered in state N₃ or B₃.

The total disassociation (off) rate from a given bead is equal to k_{off}×Nᵢ (the number of molecules currently present on the bead). However, the Lᵢ determines in part the partitioning ratio of where the dissociated molecules will land. The other factor contributing to this partition ratio is the current number of molecules present on the bead: if the capture bead becomes more and more saturated then it will be less accepting of dissociated molecules, and vice versa.

Thus, the total rate of molecules leaving a bead may be described as:

$$\frac{d {N}_{i}}{d t} = - {k}_{off} {N}_{i}$$

The probability that a molecule will land on a given bead is proportional to the spatial coupling factor as well as the available concentration of binding sites, where Nₘₐₓ is the total number of available sites on a bead, and for it to be a probability, the sum of all probabilities has to equal 1:

$${\sum }_{j} {η}_{i j} = 1 ,$$

which dictates that:

$${η}_{i j} = \frac{{t}_{ij} ( 1 - \frac{{N}_{j} + {B}_{j}}{{N}_{max}} )}{{\sum }_{i}} ,$$

where ηᵢⱼ is the probability that, given a molecule came off of location i, that it lands (and binds) at location j.

The coefficients tᵢⱼ are intended to capture the combined effect of spatial coupling (diffusion efficiency), site occupancy, as well as photobleaching to understand the transfer dynamics between a source bead, a capture bead, and loss to the main channel. It is assumed that there is no return from the main channel, and there is no return from photobleaching. Coefficients tᵢⱼ describe the spatial component only (i.e., if a molecule comes off bead i, ηᵢᵢ is the probability it diffuses back to the same bead, and ηᵢⱼ is the probability that it diffuses to state j the other bead or the main channel). In the diagram of FIG. 6A, tᵢⱼ are included in “ηᵢⱼ”. The available site concentration also factors in.

Photobleaching is also accounted for in this model with the photobleaching rate constant (k_{b}). Note that the bleached and unbleached population add up to occupy the total available sites on the bead. When accounted for this way, the binding rate to a bead will become reduced as it bleaches.

A normalization factor (Σ) is introduced to ensure that each row sums to 1:

$${\sum }_{1} = {t}_{11} ( 1 - \frac{{N}_{1} + {B}_{1}}{{N}_{max}} ) + {t}_{12} ( 1 - \frac{{N}_{2} + {B}_{2}}{{N}_{max}} ) + {t}_{13}$$

$${\sum }_{2} = {t}_{21} ( 1 - \frac{{N}_{1} + {B}_{1}}{{N}_{max}} ) + {t}_{22} ( 1 - \frac{{N}_{2} + {B}_{2}}{{N}_{max}} ) + {t}_{23}$$

$${\sum }_{3} = 1$$

Σ₃=1 as there is only one non-zero term in the set of transitions from the main channel: main channel only dissociates to itself. It is possible to simplify these expressions and eliminate two of the tᵢⱼ parameters (the unity sum criteria allows simplification to two parameter ratios for each row). Bᵢ is the number of photo-bleached molecules on bead i. A photo-bleached molecule can be present on a bead but not detectable.

Then, a matrix of all the relevant transition rates can be provided:

$$η = {k}_{off} [ \begin{matrix}{η}_{11} & {η}_{12} & {η}_{13} \\ {η}_{21} & {η}_{22} & {η}_{23} \\ 0 & 0 & 1\end{matrix} ] = {k}_{off} [ \begin{matrix}\frac{{t}_{11} ( 1 - \frac{{N}_{1} + {B}_{1}}{{N}_{max}} )}{{\sum }_{1}} & \frac{{t}_{12} ( 1 - \frac{{N}_{2} + {B}_{2}}{{N}_{max}} )}{{\sum }_{1}} & \frac{{t}_{13}}{{\sum }_{1}} \\ \frac{{t}_{21} ( 1 - \frac{{N}_{1} + {B}_{1}}{{N}_{max}} )}{{\sum }_{2}} & \frac{{t}_{22} ( 1 - \frac{{N}_{2} + {B}_{2}}{{N}_{max}} )}{{\sum }_{2}} & \frac{{t}_{23}}{{\sum }_{2}} \\ 0 & 0 & 1\end{matrix} ]$$

This matrix is then incorporated into a set of four coupled Ordinary Differential Equations:

$$\frac{d {N}_{1}}{d t} = {k}_{off} ( {N}_{2} {η}_{21} - {N}_{1} ( 1 - {η}_{11} ) ) - {k}_{b} {N}_{1}$$

$$\frac{d {N}_{2}}{d t} = {k}_{off} ( {N}_{1} {η}_{12} - {N}_{2} ( 1 - {η}_{22} ) ) - {k}_{b} {N}_{2}$$

$$\frac{d {B}_{1}}{d t} = {k}_{off} ( {B}_{2} {η}_{21} - {B}_{1} ( 1 - {η}_{11} ) ) + {k}_{b} {N}_{1}$$

$$\frac{d {B}_{2}}{d t} = {k}_{off} ( {B}_{1} {η}_{12} - {B}_{2} ( 1 - {η}_{22} ) ) + {k}_{b} {N}_{2}$$

The above equations can be solved using a simple ODE solver for the given set of parameters: tᵢⱼ (fit to the model); k_{off} (fit to the model); k_{b} (photobleaching rate comes from experimental measurement); Nₘₐₓ (fit or preferably assumed that the source bead initial brightness is Nₘₐₓ). Assuming N₁=Nₘₐₓ at time=0, and all others=0.

Then a numerical solution of the ODEs is implemented to fit to experimental data to obtain k_{off}. As result, concentrations (i.e., occupation number) for each bead is plotted over time. The number of occupations on the capture bead increases over time while the number of occupations on the source bead decreases over time. The ratio of the capture bead to the source bead in the number of occupations increases with a rising exponential whose time constant reflects k_{off} (FIG. 6B). The effect of photobleaching can be substantially cancelled out by the ratiometric measurement.

To fit the calculation to experimental data more accurately, the source and capture beads can be positioned in the chamber or sequestration pen within a predetermined range, such as an exemplary range described elsewhere herein.

In embodiments where a plurality of capture micro-objects (source beads or capture beads) are present adjacent to each other in the chamber but separate from the other type of capture micro-object (source or capture bead), the above ηᵢⱼ may account for the probabilities from all of the plurality of beads (for example, as shown in FIG. 6B). In the model described above, the tᵢⱼ can be modified by increasing the tᵢᵢ (that accounts for self-binding factors), which can in turn decrease the cross-terms, slowing down the overall rate of transfer from the source bead to the capture bead. Then, a numerical solution of the ODEs above is implemented to fit to experimental data to obtain an overall k_{off} representative of binding affinities.

Examples of obtaining characteristic k_{off} fit to the experimental data are provided in Examples 1 and 2 below.

Using the array of chambers/pens of microfluidic devices described herein (for example, those used in Example 2), multiple different assays can be performed in parallel. Examples of parameters that can be varied across the array of chambers/pens (e.g., adjacent chambers/pens or groups/increments/sequences of chambers/pens in the array of chambers/pens) could, for example, include capture beads coated with a corresponding distinct binding partners that differ across the array, source beads incubated with different cells of the array, signal-emitting moieties that differ across the array of pens, and bound molecules that differ across the array of pens.

2) Assays for Detecting Binding Kinetics for Different Binding Partners

In some embodiments, a method for assaying binding affinities of a target molecule and each of a plurality of distinct binding partners in a micro-fluidic device is provided. The micro-fluidic device comprises a flow region and a plurality of chambers that open off of the flow region. In some embodiments, the method comprises: providing the target molecule into the plurality of chambers, wherein the target molecule is labeled with a signal-emitting moiety and wherein a first plurality of capture micro-objects, each comprising a distinct binding partner, are present in the plurality of chambers; and allowing the target molecule to bind to the binding partners of the capture micro-objects of the first plurality, wherein the binding of the target molecule to the binding partners is allowed to proceed to saturation; removing unbound target molecule from the microfluidic device; providing a second plurality of capture micro-objects into the plurality of chambers, wherein each of the capture micro-objects of the second plurality comprises a corresponding distinct binding partner; detecting over a period of time a decrease in amount of target molecule bound to the capture micro-objects of the first plurality; optionally detecting over a period of time an increase in the amount of target molecule bound to the capture micro-objects of the second plurality; determining the relative binding affinities of the target molecule and each of the plurality of distinct binding partners.

In some embodiments, the binding affinities of the target molecule and each of the plurality of distinct binding partners are determined based on decreases in the amount of target molecule bound to the capture micro-objects of the first plurality over the period of time. In some embodiments, the binding affinities of the target molecule and each of the plurality of distinct binding partners are calculated ratios of (i) increases in the amount of target molecule bound to the capture micro-objects of the second plurality over the period of time to (ii) decreases in the amount of target molecule bound to the capture micro-objects of the first plurality over the period of time.

In some embodiments, first capture micro-objects comprising distinct binding partners are distinctly labeled. First capture micro-objects may be labeled with fluorescent tags or any other indicators that help visually identify the type of capture micro-object such that the specific micro-object may be moved into the chamber or sequestration pen using DEP according to the indicator or tag.

In some embodiments, the method further comprises providing the capture micro-objects of the first plurality into the plurality of chambers prior to providing the target molecule into the plurality of chambers.

In some embodiments, the plurality of chambers is a first plurality of chambers and prior to providing the first plurality of capture micro-objects into the first plurality of chambers, the method comprises disposing the first plurality of capture micro-objects into a second plurality of chambers in which the distinct binding partners are present, and allowing the binding partners to bind to the capture micro-objects of the first plurality in the second plurality of chambers.

In some embodiments, the method further comprises, prior to and/or simultaneously with allowing the binding partners to bind to the capture micro-objects of the first plurality in the second plurality of chambers: culturing a plurality of biological cells in the second plurality of chambers, wherein the plurality of biological cells secrete the binding partners.

In some embodiments, each chamber of the first plurality is adjacent to a chamber of the second plurality, and providing the first plurality of capture micro-objects into the first plurality of chambers comprises moving the capture micro-objects of the first plurality from a chamber of the second plurality into the adjacent chamber of the first plurality.

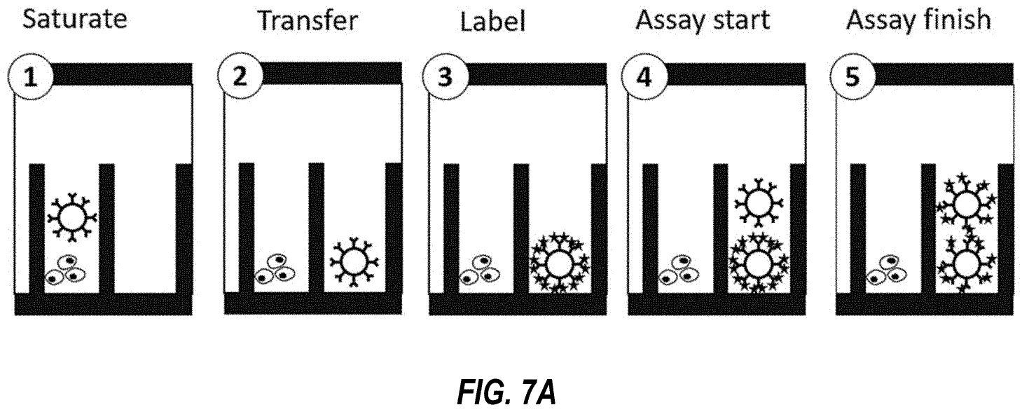

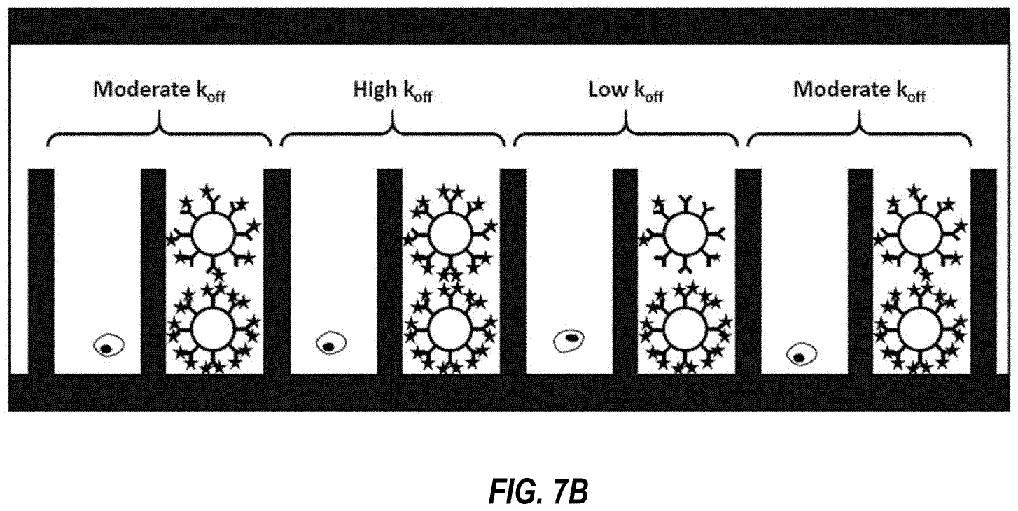

FIG. 7A shows an embodiment of the method described herein comprising: 1) culturing a plurality of biological cells in a chamber, wherein the plurality of biological cells secrete the binding partners; 2) moving the first capture micro-object (source bead) from the chamber into the adjacent chamber; 3) providing the target molecule labeled with a signal-emitting moiety into the chamber and allowing the target molecule to bind to the binding partners of the first capture micro-object, wherein the binding of the target molecule to the binding partners is allowed to proceed to saturation; 4) providing a second capture micro-object (capture bead) into the chamber; and 5) allowing the target molecule to bind to the binding partners of the second capture micro-object, and optionally the binding of the target molecule to the binding partners of the second capture micro-object is allowed to proceed to saturation.

In some embodiments, the methods described herein may be suitable for assays of binding kinetics for different antibodies specific to the same substrate. In some embodiments, the methods described herein may be suitable for assays of binding kinetics for different antibodies specific to the same substrate for cells expressing on the same device (FIG. 7B). In some embodiments, the methods described herein may be suitable for assays of binding kinetics for bispecific antibodies (e.g., antibodies with variable chain regions that differ between the two variable regions of a given antibody).

In some embodiments, the methods described herein may be provided for assays of binding kinetics for different antibodies specific to the same antigen in which the antibody on the capture bead (second capture micro-object) differs from the antibody on the source bead (first capture micro-object) for the same antigen. For example, where the antibody on the capture bead has a higher binding affinity for the antigen than the antibody on the source bead, the rate of transfer from the source bead to the capture bead would be faster than the case where the same antibody is used for both the capture bead and the source bead. In further embodiments, the antibody on the capture bead has a known binding affinity (a known k_{off} value) for the antigen, the k_{off} value for the antibody on the source bead may be obtained.

In some embodiments, a method for assaying binding affinities of a target molecule and one or more distinct binding partners for the target molecule in a micro-fluidic device is provided, wherein the micro-fluidic device comprises a flow region and a chamber that open off of the flow region, the method comprising:
providing the target molecule into the chamber, wherein the target molecule is labeled with a signal-emitting moiety and wherein a first capture micro-object comprising a first binding partner are present in the chamber; and allowing the target molecule to bind to the first binding partner of the first capture micro-object, wherein the binding of the target molecule to the first binding partner is allowed to proceed to saturation;
removing unbound target molecule from the microfluidic device;
providing a second capture micro-object into the chamber, wherein the second capture micro-object comprises a second binding partner different from the first binding partner;
detecting over a period of time a decrease in amount of target molecule bound to the first capture micro-object;
optionally detecting over the period of time an increase in the amount of target molecule bound to the second capture micro-object;
determining the relative binding affinity of the target molecule and the first binding partner based on (1) the decrease in the amount of target molecule bound to the first capture micro-object over the period of time or (2) a ratio of (i) the increase in the amount of target molecule bound to the second capture micro-object over the period of time to (ii) the decrease in the amount of target molecule bound to the first capture micro-object over the period of time.

In some embodiments, a binding partner with a known dissociation rate constant (k_{off}) is used for the second binding partner of the second capture micro-object. In some embodiments, an estimated k_{off} is supplied for the second binding partner of the second capture micro-object. In some embodiments, k_{off} is calculated for the second binding partner of the second capture micro-object based on the ratio of (i) the increase in the amount of target molecule bound to the second capture micro-object over the period of time to (ii) the decrease in the amount of target molecule bound to the first capture micro-object over the period of time. In some embodiments, the method is performed in parallel by providing a plurality of first capture micro-objects in different chambers and providing a second capture micro-object into each of the chambers, followed by performing steps of detecting and determining binding affinities for each pairing of the second capture micro-object with the first capture micro-objects comprising different first binding partners. See, e.g., embodiment 33 described elsewhere herein.

3) Coating Solutions and Coating Agents.

In some embodiments, the inner surface of the chamber or sequestration pen is treated with a coating material for linking the first and/or second capture micro-object to the inner surface prior to introducing the first and/or second capture micro-object into the chamber. In some embodiments, the first and/or second capture micro-object is covalently linked to the inner surface treated with the coating material. In some embodiments, the first and/or second capture micro-object is non-covalently linked to the inner surface treated with the coating material.

Without intending to be limited by theory, maintenance of a biological micro-object (e.g., a biological cell) within a microfluidic device (e.g., a DEP-configured and/or EW-configured microfluidic device) may be facilitated (i.e., the biological micro-object exhibits increased viability, greater expansion and/or greater portability within the microfluidic device) when at least one or more inner surfaces of the microfluidic device have been conditioned or coated so as to present a layer of organic and/or hydrophilic molecules that provides the primary interface between the microfluidic device and biological micro-object(s) maintained therein. In some embodiments, one or more of the inner surfaces of the microfluidic device (e.g. the inner surface of the electrode activation substrate of a DEP-configured microfluidic device, the cover of the microfluidic device, and/or the surfaces of the circuit material) may be treated with or modified by a coating solution and/or coating agent to generate the desired layer of organic and/or hydrophilic molecules.

The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a DEP-configured microfluidic device) are treated or “primed” with a coating solution comprising a coating agent prior to introduction of the biological micro-object(s) into the microfluidic device.

In some embodiments, at least one surface of the microfluidic device includes a coating material that provides a layer of organic and/or hydrophilic molecules suitable for maintenance and/or expansion of biological micro-object(s) (e.g. provides a conditioned surface as described below). In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials.

Coating agent/Solution. Any convenient coating agent/coating solution can be used, including but not limited to: serum or serum factors, bovine serum albumin (BSA), polymers, detergents, enzymes, and any combination thereof.

Polymer-based coating materials. The at least one inner surface may include a coating material that comprises a polymer. The polymer may be covalently or non-covalently bound (or may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein.

The polymer may include a polymer including alkylene ether moieties. A wide variety of alkylene ether containing polymers may be suitable for use in the microfluidic devices described herein. One non-limiting exemplary class of alkylene ether containing polymers are amphiphilic nonionic block copolymers which include blocks of polyethylene oxide (PEO) and polypropylene oxide (PPO) subunits in differing ratios and locations within the polymer chain. Pluronic® polymers (BASF) are block copolymers of this type and are known in the art to be suitable for use when in contact with living cells. The polymers may range in average molecular mass M_{w} from about 2000 Da to about 20 KDa. In some embodiments, the PEO-PPO block copolymer can have a hydrophilic-lipophilic balance (HLB) greater than about 10 (e.g. 12-18). Specific Pluronic® polymers useful for yielding a coated surface include Pluronic® L44, L64, P85, and F127 (including F127NF). Another class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da.

In other embodiments, the coating material may include a polymer containing carboxylic acid moieties. The carboxylic acid subunit may be an alkyl, alkenyl or aromatic moiety containing subunit. One non-limiting example is polylactic acid (PLA). In other embodiments, the coating material may include a polymer containing phosphate moieties, either at a terminus of the polymer backbone or pendant from the backbone of the polymer. In yet other embodiments, the coating material may include a polymer containing sulfonic acid moieties. The sulfonic acid subunit may be an alkyl, alkenyl or aromatic moiety containing subunit. One non-limiting example is polystyrene sulfonic acid (PSSA) or polyanethole sulfonic acid. In further embodiments, the coating material may include a polymer including amine moieties. The polyamino polymer may include a natural polyamine polymer or a synthetic polyamine polymer. Examples of natural polyamines include spermine, spermidine, and putrescine.

In other embodiments, the coating material may include a polymer containing saccharide moieties. In a non-limiting example, polysaccharides such as xanthan gum or dextran may be suitable to form a material which may reduce or prevent cell sticking in the microfluidic device. For example, a dextran polymer having a size about 3 kDa may be used to provide a coating material for a surface within a microfluidic device.

In other embodiments, the coating material may include a polymer containing nucleotide moieties, i.e. a nucleic acid, which may have ribonucleotide moieties or deoxyribonucleotide moieties, providing a polyelectrolyte surface. The nucleic acid may contain only natural nucleotide moieties or may contain unnatural nucleotide moieties which comprise nucleobase, ribose or phosphate moiety analogs such as 7-deazaadenine, pentose, methyl phosphonate or phosphorothioate moieties without limitation.

In yet other embodiments, the coating material may include a polymer containing amino acid moieties. The polymer containing amino acid moieties may include a natural amino acid containing polymer or an unnatural amino acid containing polymer, either of which may include a peptide, a polypeptide or a protein. In one non-limiting example, the protein may be bovine serum albumin (BSA) and/or serum (or a combination of multiple different sera) comprising albumin and/or one or more other similar proteins as coating agents. The serum can be from any convenient source, including but not limited to fetal calf serum, sheep serum, goat serum, horse serum, and the like. In certain embodiments, BSA in a coating solution is present in a concentration from about 1 mg/mL to about 100 mg/mL, including 5 mg/mL, 10 mg/mL, 20 mg/mL, 30 mg/mL, 40 mg/mL, 50 mg/mL, 60 mg/mL, 70 mg/mL, 80 mg/mL, 90 mg/mL, or more or anywhere in between. In certain embodiments, serum in a coating solution may be present in a concentration of about 20% (v/v) to about 50% v/v, including 25%, 30%, 35%, 40%, 45%, or more or anywhere in between. In some embodiments, BSA may be present as a coating agent in a coating solution at 5 mg/mL, whereas in other embodiments, BSA may be present as a coating agent in a coating solution at 70 mg/mL. In certain embodiments, serum is present as a coating agent in a coating solution at 30%. In some embodiments, an extracellular matrix (ECM) protein may be provided within the coating material for optimized cell adhesion to foster cell growth. A cell matrix protein, which may be included in a coating material, can include, but is not limited to, a collagen, an elastin, an RGD-containing peptide (e.g. a fibronectin), or a laminin. In yet other embodiments, growth factors, cytokines, hormones or other cell signaling species may be provided within the coating material of the microfluidic device.

In some embodiments, the coating material may include a polymer containing more than one of alkylene oxide moieties, carboxylic acid moieties, sulfonic acid moieties, phosphate moieties, saccharide moieties, nucleotide moieties, or amino acid moieties. In other embodiments, the polymer conditioned surface may include a mixture of more than one polymer each having alkylene oxide moieties, carboxylic acid moieties, sulfonic acid moieties, phosphate moieties, saccharide moieties, nucleotide moieties, and/or amino acid moieties, which may be independently or simultaneously incorporated into the coating material.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells.

The covalently linked molecules include a linking group, wherein the linking group is covalently linked to one or more surfaces of the microfluidic device, as described below. The linking group is also covalently linked to a moiety configured to provide a layer of organic and/or hydrophilic molecules suitable for maintenance/expansion of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); azides; amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, a substituted alkyl moiety, such as a fluoroalkyl moiety (including but not limited to a perfluoroalkyl moiety), azide moiety; amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may be any of the moieties described above.

In some embodiments, the covalently linked alkyl moiety may comprises carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may include at least one alkylene oxide moiety, and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. Exemplary reactive pairing moieties may include aldehyde, alkyne or halo moieties. A polysaccharide may be modified in a random fashion, wherein each of the saccharide monomers may be modified or only a portion of the saccharide monomers within the polysaccharide are modified to provide a reactive pairing moiety that may be coupled directly or indirectly to a surface. One exemplar may include a dextran polysaccharide, which may be coupled indirectly to a surface via an unbranched linker.

The covalently linked moiety may include one or more amino groups. The amino group may be a substituted amine moiety, guanidine moiety, nitrogen-containing heterocyclic moiety or heteroaryl moiety. The amino containing moieties may have structures permitting pH modification of the environment within the microfluidic device, and optionally, within the sequestration pens and/or flow regions (e.g., channels).

The coating material providing a conditioned surface may comprise only one kind of covalently linked moiety or may include more than one different kind of covalently linked moiety. For example, the fluoroalkyl conditioned surfaces (including perfluoroalkyl) may have a plurality of covalently linked moieties which are all the same, e.g., having the same linking group and covalent attachment to the surface, the same overall length, and the same number of fluoromethylene units comprising the fluoroalkyl moiety. Alternatively, the coating material may have more than one kind of covalently linked moiety attached to the surface. For example, the coating material may include molecules having covalently linked alkyl or fluoroalkyl moieties having a specified number of methylene or fluoromethylene units and may further include a further set of molecules having charged moieties covalently attached to an alkyl or fluoroalkyl chain having a greater number of methylene or fluoromethylene units, which may provide capacity to present bulkier moieties at the coated surface. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. In another example, the covalently linked moieties may provide a zwitterionic surface presenting alternating charges in a random fashion on the surface.

Conditioned surface properties. Aside from the composition of the conditioned surface, other factors such as physical thickness of the hydrophobic material can impact DEP force. Various factors can alter the physical thickness of the conditioned surface, such as the manner in which the conditioned surface is formed on the substrate (e.g. vapor deposition, liquid phase deposition, spin coating, flooding, and electrostatic coating). In some embodiments, the conditioned surface has a thickness of about 1 nm to about 10 nm; about 1 nm to about 7 nm; about 1 nm to about 5 nm; or any individual value therebetween. In other embodiments, the conditioned surface formed by the covalently linked moieties may have a thickness of about 10 nm to about 50 nm. In various embodiments, the conditioned surface prepared as described herein has a thickness of less than 10 nm. In some embodiments, the covalently linked moieties of the conditioned surface may form a monolayer when covalently linked to the surface of the microfluidic device (e.g., a DEP configured substrate surface) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device.

In various embodiments, the coating material providing a conditioned surface of the microfluidic device may provide desirable electrical properties. Without intending to be limited by theory, one factor that impacts robustness of a surface coated with a particular coating material is intrinsic charge trapping. Different coating materials may trap electrons, which can lead to breakdown of the coating material. Defects in the coating material may increase charge trapping and lead to further breakdown of the coating material. Similarly, different coating materials have different dielectric strengths (i.e. the minimum applied electric field that results in dielectric breakdown), which may impact charge trapping. In certain embodiments, the coating material can have an overall structure (e.g., a densely-packed monolayer structure) that reduces or limits that amount of charge trapping.

In addition to its electrical properties, the conditioned surface may also have properties that are beneficial in use with biological molecules. For example, a conditioned surface that contains fluorinated (or perfluorinated) carbon chains may provide a benefit relative to alkyl-terminated chains in reducing the amount of surface fouling. Surface fouling, as used herein, refers to the amount of indiscriminate material deposition on the surface of the microfluidic device, which may include permanent or semi-permanent deposition of biomaterials such as protein and its degradation products, nucleic acids and respective degradation products and the like.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, as is described below. Alternatively, the covalently linked coating material may be formed in a two-part sequence by coupling the moiety configured to provide a layer of organic and/or hydrophilic molecules suitable for maintenance/expansion of biological micro-object(s) to a surface modifying ligand that itself has been covalently linked to the surface.

Methods of preparing a covalently linked coating material. In some embodiments, a coating material that is covalently linked to the surface of a microfluidic device (e.g., including at least one surface of the sequestration pens and/or flow regions) has a structure of Formula 1 or Formula 2. When the coating material is introduced to the surface in one step, it has a structure of Formula 1, while when the coating material is introduced in a multiple step process, it has a structure of Formula 2.

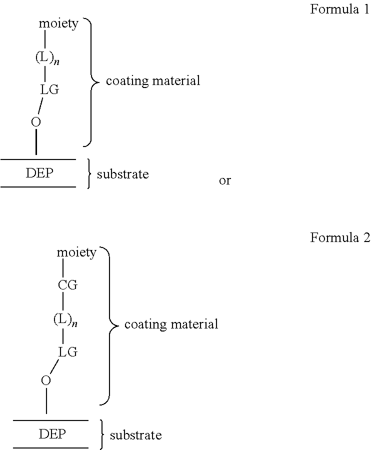

The coating material may be linked covalently to oxides of the surface of a DEP-configured or EW-configured substrate. The DEP- or EW-configured substrate may comprise silicon, silicon oxide, alumina, or hafnium oxide. Oxides may be present as part of the native chemical structure of the substrate or may be introduced as discussed below.

The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the backbone of the linker L may include 10 to 20 atoms. In other embodiments, the backbone of the linker L may include about 5 atoms to about 200 atoms; about 10 atoms to about 80 atoms; about 10 atoms to about 50 atoms; or about 10 atoms to about 40 atoms. In some embodiments, the backbone atoms are all carbon atoms.

In some embodiments, the moiety configured to provide a layer of organic and/or hydrophilic molecules suitable for maintenance/expansion of biological micro-object(s) may be added to the surface of the substrate in a multi-step process, and has a structure of Formula 2, as shown above. The moiety may be any of the moieties described above.

In some embodiments, the coupling group CG represents the resultant group from reaction of a reactive moiety Rₓ and a reactive pairing moiety Rₚₓ (i.e., a moiety configured to react with the reactive moiety Rₓ). For example, one typical coupling group CG may include a carboxamidyl group, which is the result of the reaction of an amino group with a derivative of a carboxylic acid, such as an activated ester, an acid chloride or the like. Other CG may include a triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. The coupling group CG may be located at the second end (i.e., the end proximal to the moiety configured to provide a layer of organic and/or hydrophilic molecules suitable for maintenance/expansion of biological micro-object(s) in the microfluidic device) of linker L, which may include any combination of elements as described above. In some other embodiments, the coupling group CG may interrupt the backbone of the linker L. When the coupling group CG is triazolylene, it may be the product resulting from a Click coupling reaction and may be further substituted (e.g., a dibenzocylcooctenyl fused triazolylene group).

In some embodiments, the coating material (or surface modifying ligand) is deposited on the inner surfaces of the microfluidic device using chemical vapor deposition. The vapor deposition process can be optionally improved, for example, by pre-cleaning the cover 110, the microfluidic circuit material 116, and/or the substrate (e.g., the inner surface 208 of the electrode activation substrate 206 of a DEP-configured substrate, or a dielectric layer of the support structure 104 of an EW-configured substrate), by exposure to a solvent bath, sonication or a combination thereof. Alternatively, or in addition, such pre-cleaning can include treating the cover 110, the microfluidic circuit material 116, and/or the substrate in an oxygen plasma cleaner, which can remove various impurities, while at the same time introducing an oxidized surface (e.g. oxides at the surface, which may be covalently modified as described herein). Alternatively, liquid-phase treatments, such as a mixture of hydrochloric acid and hydrogen peroxide or a mixture of sulfuric acid and hydrogen peroxide (e.g., piranha solution, which may have a ratio of sulfuric acid to hydrogen peroxide from about 3:1 to about 7:1) may be used in place of an oxygen plasma cleaner.

In some embodiments, vapor deposition is used to coat the inner surfaces of the microfluidic device 200 after the microfluidic device 200 has been assembled to form an enclosure 102 defining a microfluidic circuit 120. Without intending to be limited by theory, depositing such a coating material on a fully-assembled microfluidic circuit 120 may be beneficial in preventing delamination caused by a weakened bond between the microfluidic circuit material 116 and the electrode activation substrate 206 dielectric layer and/or the cover 110. In embodiments where a two-step process is employed the surface modifying ligand may be introduced via vapor deposition as described above, with subsequent introduction of the moiety configured provide a layer of organic and/or hydrophilic molecules suitable for maintenance/expansion of biological micro-object(s). The subsequent reaction may be performed by exposing the surface modified microfluidic device to a suitable coupling reagent in solution.

FIG. 3 depicts a cross-sectional views of a microfluidic device 290 having an exemplary covalently linked coating material providing a conditioned surface. As illustrated, the coating materials 298 (shown schematically) can comprise a monolayer of densely-packed molecules covalently bound to both the inner surface 294 of a base 286, which may be a DEP substrate, and the inner surface 292 of a cover 288 of the microfluidic device 290. The coating material 298 can be disposed on substantially all inner surfaces 294, 292 proximal to, and facing inwards towards, the enclosure 284 of the microfluidic device 290, including, in some embodiments and as discussed above, the surfaces of microfluidic circuit material (not shown) used to define circuit elements and/or structures within the microfluidic device 290. In alternate embodiments, the coating material 298 can be disposed on only one or some of the inner surfaces of the microfluidic device 290.

In the embodiment shown in FIG. 3, the coating material 298 can include a monolayer of organosiloxane molecules, each molecule covalently bonded to the inner surfaces 292, 294 of the microfluidic device 290 via a siloxy linker 296. Any of the above-discussed coating materials 298 can be used (e.g. an alkyl-terminated, a fluoroalkyl terminated moiety, a PEG-terminated moiety, a dextran terminated moiety, or a terminal moiety containing positive or negative charges for the organosiloxy moieties), where the terminal moiety is disposed at its enclosure-facing terminus (i.e. the portion of the monolayer of the coating material 298 that is not bound to the inner surfaces 292, 294 and is proximal to the enclosure 284).

In other embodiments, the coating material 298 used to coat the inner surface(s) 292, 294 of the microfluidic device 290 can include anionic, cationic, or zwitterionic moieties, or any combination thereof. Without intending to be limited by theory, by presenting cationic moieties, anionic moieties, and/or zwitterionic moieties at the inner surfaces of the enclosure 284 of the microfluidic circuit 120, the coating material 298 can form strong hydrogen bonds with water molecules such that the resulting water of hydration acts as a layer (or “shield”) that separates the biological micro-objects from interactions with non-biological molecules (e.g., the silicon and/or silicon oxide of the substrate). In addition, in embodiments in which the coating material 298 is used in conjunction with coating agents, the anions, cations, and/or zwitterions of the coating material 298 can form ionic bonds with the charged portions of non-covalent coating agents (e.g. proteins in solution) that are present in a medium 180 (e.g. a coating solution) in the enclosure 284.

In still other embodiments, the coating material may comprise or be chemically modified to present a hydrophilic coating agent at its enclosure-facing terminus. In some embodiments, the coating material may include an alkylene ether containing polymer, such as PEG. In some embodiments, the coating material may include a polysaccharide, such as dextran. Like the charged moieties discussed above (e.g., anionic, cationic, and zwitterionic moieties), the hydrophilic coating agent can form strong hydrogen bonds with water molecules such that the resulting water of hydration acts as a layer (or “shield”) that separates the biological micro-objects from interactions with non-biological molecules (e.g., the silicon and/or silicon oxide of the substrate).

Further details of appropriate coating treatments and modifications may be found at US Application Publication No. 2016/0312165, the content of which is incorporated by reference in its entirety.

Additional System Components for Maintenance of Viability of Cells within the Sequestration Pens of the Microfluidic Device.

In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

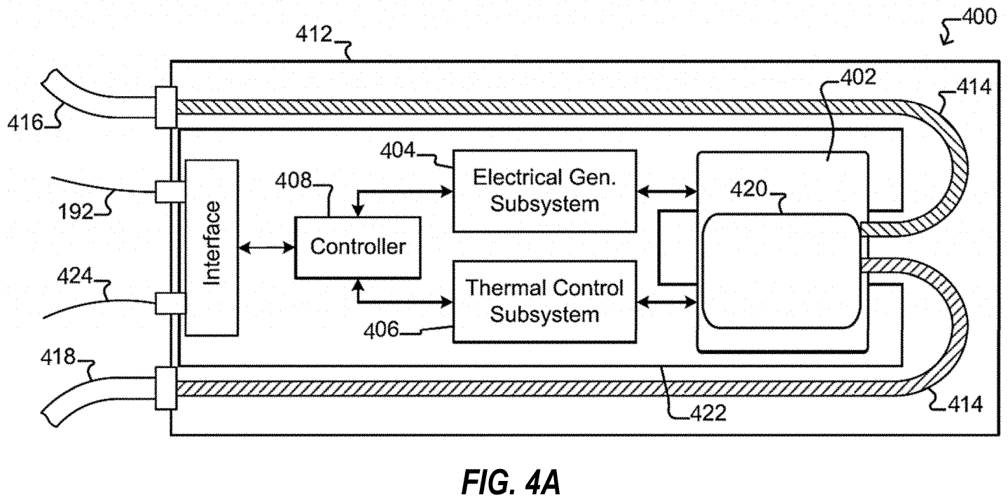

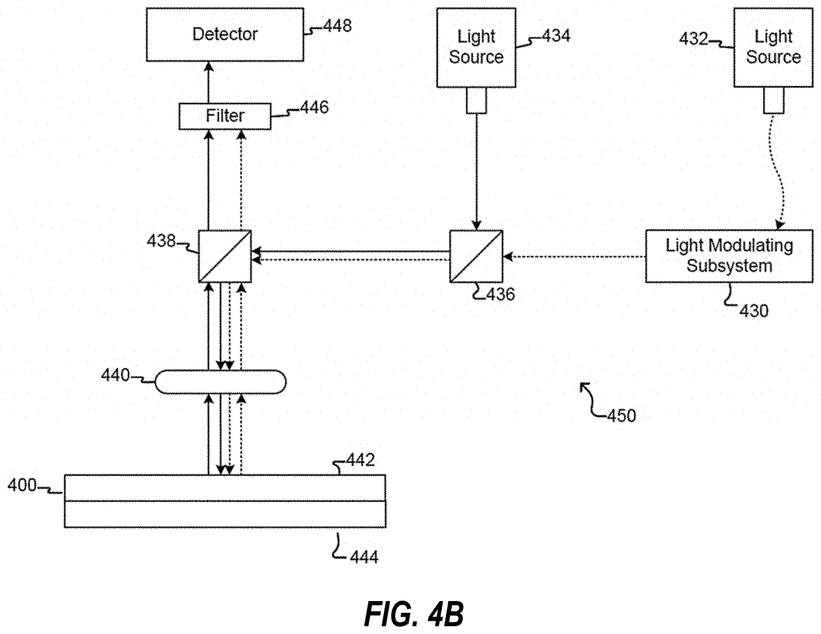

FIGS. 4A through 4B shows various embodiments of system 150 which can be used to operate and observe microfluidic devices (e.g. 100, 200, 230, 250, 280, 290, 300) according to the present disclosure. As illustrated in FIG. 4A, the system 150 can include a structure (“nest”) 400 configured to hold a microfluidic device 100 (not shown), or any other microfluidic device described herein. The nest 400 can include a socket 402 capable of interfacing with the microfluidic device 420 (e.g., an optically-actuated electrokinetic device 100) and providing electrical connections from power source 192 to microfluidic device 420. The nest 400 can further include an integrated electrical signal generation subsystem 404. The electrical signal generation subsystem 404 can be configured to supply a biasing voltage to socket 402 such that the biasing voltage is applied across a pair of electrodes in the microfluidic device 420 when it is being held by socket 402. Thus, the electrical signal generation subsystem 404 can be part of power source 192. The ability to apply a biasing voltage to microfluidic device 420 does not mean that a biasing voltage will be applied at all times when the microfluidic device 420 is held by the socket 402. Rather, in most cases, the biasing voltage will be applied intermittently, e.g., only as needed to facilitate the generation of electrokinetic forces, such as dielectrophoresis or electro-wetting, in the microfluidic device 420.

As illustrated in FIG. 4A, the nest 400 can include a printed circuit board assembly (PCBA) 422. The electrical signal generation subsystem 404 can be mounted on and electrically integrated into the PCBA 422. The exemplary support includes socket 402 mounted on PCBA 422, as well.

Typically, the electrical signal generation subsystem 404 will include a waveform generator (not shown). The electrical signal generation subsystem 404 can further include an oscilloscope (not shown) and/or a waveform amplification circuit (not shown) configured to amplify a waveform received from the waveform generator. The oscilloscope, if present, can be configured to measure the waveform supplied to the microfluidic device 420 held by the socket 402. In certain embodiments, the oscilloscope measures the waveform at a location proximal to the microfluidic device 420 (and distal to the waveform generator), thus ensuring greater accuracy in measuring the waveform actually applied to the device. Data obtained from the oscilloscope measurement can be, for example, provided as feedback to the waveform generator, and the waveform generator can be configured to adjust its output based on such feedback. An example of a suitable combined waveform generator and oscilloscope is the Red Pitaya™.

In certain embodiments, the nest 400 further comprises a controller 408, such as a microprocessor used to sense and/or control the electrical signal generation subsystem 404. Examples of suitable microprocessors include the Arduino™ microprocessors, such as the Arduino Nano™. The controller 408 may be used to perform functions and analysis or may communicate with an external master controller 154 (shown in FIG. 1A) to perform functions and analysis. In the embodiment illustrated in FIG. 3A the controller 408 communicates with a master controller 154 through an interface 410 (e.g., a plug or connector).

In some embodiments, the nest 400 can comprise an electrical signal generation subsystem 404 comprising a Red Pitaya™ waveform generator/oscilloscope unit (“Red Pitaya unit”) and a waveform amplification circuit that amplifies the waveform generated by the Red Pitaya unit and passes the amplified voltage to the microfluidic device 100. In some embodiments, the Red Pitaya unit is configured to measure the amplified voltage at the microfluidic device 420 and then adjust its own output voltage as needed such that the measured voltage at the microfluidic device 420 is the desired value. In some embodiments, the waveform amplification circuit can have a +6.5V to −6.5V power supply generated by a pair of DC-DC converters mounted on the PCBA 422, resulting in a signal of up to 13 Vpp at the microfluidic device 100.

As illustrated in FIG. 4A, the support structure 400 (e.g., nest) can further include a thermal control subsystem 406. The thermal control subsystem 406 can be configured to regulate the temperature of microfluidic device 420 held by the support structure 400. For example, the thermal control subsystem 406 can include a Peltier thermoelectric device (not shown) and a cooling unit (not shown). The Peltier thermoelectric device can have a first surface configured to interface with at least one surface of the microfluidic device 420. The cooling unit can be, for example, a cooling block (not shown), such as a liquid-cooled aluminum block. A second surface of the Peltier thermoelectric device (e.g., a surface opposite the first surface) can be configured to interface with a surface of such a cooling block. The cooling block can be connected to a fluidic path 414 configured to circulate cooled fluid through the cooling block. In the embodiment illustrated in FIG. 4A, the support structure 400 comprises an inlet 416 and an outlet 418 to receive cooled fluid from an external reservoir (not shown), introduce the cooled fluid into the fluidic path 414 and through the cooling block, and then return the cooled fluid to the external reservoir. In some embodiments, the Peltier thermoelectric device, the cooling unit, and/or the fluidic path 414 can be mounted on a casing 412 of the support structure 400. In some embodiments, the thermal control subsystem 406 is configured to regulate the temperature of the Peltier thermoelectric device so as to achieve a target temperature for the microfluidic device 420. Temperature regulation of the Peltier thermoelectric device can be achieved, for example, by a thermoelectric power supply, such as a Pololu™ thermoelectric power supply (Pololu Robotics and Electronics Corp.). The thermal control subsystem 406 can include a feedback circuit, such as a temperature value provided by an analog circuit. Alternatively, the feedback circuit can be provided by a digital circuit.

In some embodiments, the nest 400 can include a thermal control subsystem 406 with a feedback circuit that is an analog voltage divider circuit (not shown) which includes a resistor (e.g., with resistance 1 kOhm+/−0.1%, temperature coefficient+/−0.02 ppm/CO) and a NTC thermistor (e.g., with nominal resistance 1 kOhm+/−0.01%). In some instances, the thermal control subsystem 406 measures the voltage from the feedback circuit and then uses the calculated temperature value as input to an on-board PID control loop algorithm. Output from the PID control loop algorithm can drive, for example, both a directional and a pulse-width-modulated signal pin on a Pololu™ motor drive (not shown) to actuate the thermoelectric power supply, thereby controlling the Peltier thermoelectric device.

The nest 400 can include a serial port 424 which allows the microprocessor of the controller 408 to communicate with an external master controller 154 via the interface 410 (not shown). In addition, the microprocessor of the controller 408 can communicate (e.g., via a Plink tool (not shown)) with the electrical signal generation subsystem 404 and thermal control subsystem 406. Thus, via the combination of the controller 408, the interface 410, and the serial port 424, the electrical signal generation subsystem 404 and the thermal control subsystem 406 can communicate with the external master controller 154. In this manner, the master controller 154 can, among other things, assist the electrical signal generation subsystem 404 by performing scaling calculations for output voltage adjustments. A Graphical User Interface (GUI) (not shown) provided via a display device 170 coupled to the external master controller 154, can be configured to plot temperature and waveform data obtained from the thermal control subsystem 406 and the electrical signal generation subsystem 404, respectively. Alternatively, or in addition, the GUI can allow for updates to the controller 408, the thermal control subsystem 406, and the electrical signal generation subsystem 404.

As discussed above, system 150 can include an imaging device 194. In some embodiments, the imaging device 194 comprises a light modulating subsystem 430 (See FIG. 4B). The light modulating subsystem 430 can include a digital mirror device (DMD) or a microshutter array system (MSA), either of which can be configured to receive light from a light source 432 and transmits a subset of the received light into an optical train of microscope 450. Alternatively, the light modulating subsystem 430 can include a device that produces its own light (and thus dispenses with the need for a light source 432), such as an organic light emitting diode display (OLED), a liquid crystal on silicon (LCOS) device, a ferroelectric liquid crystal on silicon device (FLCOS), or a transmissive liquid crystal display (LCD). The light modulating subsystem 430 can be, for example, a projector. Thus, the light modulating subsystem 430 can be capable of emitting both structured and unstructured light. In certain embodiments, imaging module 164 and/or motive module 162 of system 150 can control the light modulating subsystem 430.

In certain embodiments, the imaging device 194 further comprises a microscope 450. In such embodiments, the nest 400 and light modulating subsystem 430 can be individually configured to be mounted on the microscope 450. The microscope 450 can be, for example, a standard research-grade light microscope or fluorescence microscope. Thus, the nest 400 can be configured to be mounted on the stage 444 of the microscope 450 and/or the light modulating subsystem 430 can be configured to mount on a port of microscope 450. In other embodiments, the nest 400 and the light modulating subsystem 430 described herein can be integral components of microscope 450.

In certain embodiments, the microscope 450 can further include one or more detectors 448. In some embodiments, the detector 448 is controlled by the imaging module 164. The detector 448 can include an eye piece, a charge-coupled device (CCD), a camera (e.g., a digital camera), or any combination thereof. If at least two detectors 448 are present, one detector can be, for example, a fast-frame-rate camera while the other detector can be a high sensitivity camera. Furthermore, the microscope 450 can include an optical train configured to receive reflected and/or emitted light from the microfluidic device 420 and focus at least a portion of the reflected and/or emitted light on the one or more detectors 448. The optical train of the microscope can also include different tube lenses (not shown) for the different detectors, such that the final magnification on each detector can be different.

In certain embodiments, imaging device 194 is configured to use at least two light sources. For example, a first light source 432 can be used to produce structured light (e.g., via the light modulating subsystem 430) and a second light source 434 can be used to provide unstructured light. The first light source 432 can produce structured light for optically-actuated electrokinesis and/or fluorescent excitation, and the second light source 434 can be used to provide bright field illumination. In these embodiments, the motive module 164 can be used to control the first light source 432 and the imaging module 164 can be used to control the second light source 434. The optical train of the microscope 450 can be configured to (1) receive structured light from the light modulating subsystem 430 and focus the structured light on at least a first region in a microfluidic device, such as an optically-actuated electrokinetic device, when the device is being held by the nest 400, and (2) receive reflected and/or emitted light from the microfluidic device and focus at least a portion of such reflected and/or emitted light onto detector 448. The optical train can be further configured to receive unstructured light from a second light source and focus the unstructured light on at least a second region of the microfluidic device, when the device is held by the nest 400. In certain embodiments, the first and second regions of the microfluidic device can be overlapping regions. For example, the first region can be a subset of the second region. In other embodiments, the second light source 434 may additionally or alternatively include a laser, which may have any suitable wavelength of light. The representation of the optical system shown in FIG. 4B is a schematic representation only, and the optical system may include additional filters, notch filters, lenses and the like. When the second light source 434 includes one or more light source(s) for brightfield and/or fluorescent excitation, as well as laser illumination the physical arrangement of the light source(s) may vary from that shown in FIG. 4B, and the laser illumination may be introduced at any suitable physical location within the optical system. The schematic locations of light source 434 and light source 432/light modulating subsystem 430 may be interchanged as well.

In FIG. 4B, the first light source 432 is shown supplying light to a light modulating subsystem 430, which provides structured light to the optical train of the microscope 450 of system 455 (not shown). The second light source 434 is shown providing unstructured light to the optical train via a beam splitter 436. Structured light from the light modulating subsystem 430 and unstructured light from the second light source 434 travel from the beam splitter 436 through the optical train together to reach a second beam splitter (or dichroic filter 438, depending on the light provided by the light modulating subsystem 430), where the light gets reflected down through the objective 436 to the sample plane 442. Reflected and/or emitted light from the sample plane 442 then travels back up through the objective 440, through the beam splitter and/or dichroic filter 438, and to a dichroic filter 446. Only a fraction of the light reaching dichroic filter 446 passes through and reaches the detector 448.

In some embodiments, the second light source 434 emits blue light. With an appropriate dichroic filter 446, blue light reflected from the sample plane 442 is able to pass through dichroic filter 446 and reach the detector 448. In contrast, structured light coming from the light modulating subsystem 430 gets reflected from the sample plane 442, but does not pass through the dichroic filter 446. In this example, the dichroic filter 446 is filtering out visible light having a wavelength longer than 495 nm. Such filtering out of the light from the light modulating subsystem 430 would only be complete (as shown) if the light emitted from the light modulating subsystem did not include any wavelengths shorter than 495 nm. In practice, if the light coming from the light modulating subsystem 430 includes wavelengths shorter than 495 nm (e.g., blue wavelengths), then some of the light from the light modulating subsystem would pass through filter 446 to reach the detector 448. In such an embodiment, the filter 446 acts to change the balance between the amount of light that reaches the detector 448 from the first light source 432 and the second light source 434. This can be beneficial if the first light source 432 is significantly stronger than the second light source 434. In other embodiments, the second light source 434 can emit red light, and the dichroic filter 446 can filter out visible light other than red light (e.g., visible light having a wavelength shorter than 650 nm).

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments, in all respects, are meant to be illustrative only and should not be construed to be limiting in any manner. Furthermore, where reference is made herein to a list of elements (e.g., elements a, b, c), such reference is intended to include any one of the listed elements by itself, any combination of less than all of the listed elements, and/or a combination of all of the listed elements. Also, as used herein, the terms a, an, and one may each be interchangeable with the terms at least one and one or more. It should also be noted, that while the term step is used herein, that term may be used to simply draw attention to different portions of the described methods and is not meant to delineate a starting point or a stopping point for any portion of the methods, or to be limiting in any other way.

### EXEMPLARY EMBODIMENTS

Exemplary embodiments provided in accordance with the presently disclosed subject matter include, but are not limited to, the embodiments and the following embodiments:

Embodiment 1. A method for assaying a binding affinity between a first molecule and a second molecule in a micro-fluidic device, wherein the micro-fluidic device comprises a flow region and a chamber that opens off of the flow region, the method comprising:
providing the second molecule into the chamber, wherein the second molecule is labeled with a signal-emitting moiety and a first capture micro-object comprising the first molecule is present in the chamber, and allowing the second molecule to bind to the first molecule of the first capture micro-object, wherein the binding of the second molecule to the first molecule is allowed to proceed to saturation;
removing unbound second molecule from the microfluidic device;
providing a second capture micro-object into the chamber, wherein the second capture micro-object comprises a third molecule which specifically binds to the second molecule; detecting over a period of time a decrease in an amount of second molecule bound to the first capture micro-object;
optionally detecting over the period of time an increase in the amount of second molecule bound to the second capture micro-object; and
determining a relative binding affinity between the first molecule and the second molecule based on one of the following:
i. the decrease in the amount of second molecule bound to the first capture micro-object over the period of time; or
ii. a ratio of (i) the increase in the amount of second molecule bound to the second capture micro-object over the period of time to (ii) the decrease in the amount of second molecule bound to the first capture micro-object over the period of time.

Embodiment 2. A method for assaying a binding affinity between a first molecule and a second molecule in a micro-fluidic device, wherein the micro-fluidic device comprises a flow region, a chamber that opens off of the flow region, the method comprising:
providing a second molecule labeled with a signal-emitting moiety into the chamber, wherein a first capture micro-object comprising the first molecule is present in the chamber, and allowing the second molecule to bind to the first molecule of the first capture micro-object, wherein the binding of the second molecule to the first molecule is allowed to proceed to saturation;
removing unbound second molecule from the microfluidic device;
detecting over a period of time a decrease in the amount of the second molecule bound to the first capture micro-object; and
determining a relative binding affinity between the first molecule and the second molecule based on the decrease in the amount of the second molecule bound to the first capture micro-object over the period of time.

Embodiment 3. The method of any one of the preceding embodiments, wherein determining the relative binding affinity between the first molecule and the second molecule comprises calculating a dissociation rate constant (k_{off}) for the first and second molecules.

Embodiment 4. The method of any one of the preceding embodiments, wherein determining the relative binding affinity between the first molecule and the second molecule comprises dividing the dissociation rate constant (k_{off}) for the first and second molecules by an association rate constant (kₒₙ).

Embodiment 5. The method of embodiment 4, wherein kₒₙ is an estimated value (e.g., is estimated based on known association rate constants for molecules similar to the first and second molecules).

Embodiment 6. The method of embodiment 4 or embodiment 5, wherein a kₒₙ value in the range of about 1×10⁶ to about 1×10⁷ M⁻¹ s⁻¹ is used.

Embodiment 7. The method of any one of embodiments 3-6, wherein the k_{off} is determined to be in the range of about 1×10⁻⁵ to about 1×10⁻³ to s⁻¹.

Embodiment 8. The method of any one of embodiments 1 to 7, wherein providing the second molecule into the chamber comprises:
flowing a solution comprising the second molecule through the flow path in the microfluidic device; and
allowing the second molecule to diffuse into the chamber.

Embodiment 9. The method of any one of embodiments 1 to 8, further comprising, prior to providing the second molecule into the chamber, providing the first capture micro-object into the chamber.

Embodiment 10. The method of embodiment 9, wherein the chamber is a first chamber and wherein prior to providing the first capture micro-object into the chamber, the method comprises disposing a capture micro-object into a second chamber in which the first molecule is present, and allowing the first molecule to bind to the capture micro-object in the second chamber and thereby generate the first capture micro-object, optionally wherein the second chamber is adjacent to the first chamber.

Embodiment 11. The method of embodiment 10, further comprising, prior to and/or simultaneously with allowing the first molecule to bind to the capture micro-object in the second chamber:
culturing one or more biological cells in the second chamber, wherein the one or more biological cells secrete the first molecule.

Embodiment 12. The method of any one of embodiments 1 to 11, wherein the first molecule is an antibody or an antigen-binding fragment thereof.

Embodiment 13. The method of any one of embodiments 1 and 3-12, wherein the third molecule is an antibody or an antigen-binding fragment thereof.

Embodiment 14. The method of any one of embodiments 1 and 3-13, wherein the first molecule binds to a first epitope on the second molecule, wherein the third molecule binds to a second epitope on the second molecule, and wherein the first epitope and the second epitope are substantially the same.

Embodiment 15. The method of any one of embodiments 1 and 3 to 14, wherein the first molecule binds to a first epitope on the second molecule, wherein the third molecule binds to a second epitope on the second molecule, and wherein the first epitope and the second epitope are different.

Embodiment 16. The method of any one of embodiments 1 and 3 to 15, wherein the third molecule is substantially identical to the first molecule.

Embodiment 17. The method of any one of embodiments 1 and 3 to 15, wherein the third molecule is different than the first molecule.

Embodiment 18. The method of any one of embodiments 1, 3 to 15, or 17, wherein the first molecule binds the second molecule with a first dissociation rate constant k_{off}¹, wherein the third molecule binds the second molecule with a second dissociation rate constant k_{off}² equal to or up to one order of magnitude greater than the first dissociation rate constant k_{off}¹; or wherein the first molecule binds the second molecule with a first dissociation constant K_{d}¹, wherein the third molecule binds the second molecule with a second dissociation constant K_{d}² equal to or up to one order of magnitude greater than the first dissociation constant K_{d}¹.

Embodiment 19. The method of any one of embodiments 1 to 13 or 18, wherein the first molecule binds the second molecule with a first dissociation rate constant k_{off}¹, wherein the third molecule binds the second molecule with a second dissociation rate constant k_{off}² within a factor of 5, 4, 3, 2, or 1.5 of the first dissociation rate constant k_{off}¹; or wherein the first molecule binds the second molecule with a first dissociation constant K_{d}¹, wherein the third molecule binds the second molecule with a second dissociation constant K_{d}² within a factor of 5, 4, 3, 2, or 1.5 of the first dissociation constant K_{d}¹.

Embodiment 20. The method of any one of embodiments 1-19, wherein the second molecule is an antigen.

Embodiment 21. The method of any one of embodiments 20, wherein the antigen is an antigen expressed by a pathogenic agent (e.g., a virus, a bacterium, a cancer cell, or the like).

Embodiment 22. The method of any one of embodiments 21, wherein the antigen is a peptide, an extracellular signaling molecule, or a cell-surface protein.

Embodiment 23. The method of any one of embodiments 1-22, wherein the signal-emitting moiety comprises a fluorophore.

Embodiment 24. The method of any one of embodiments 1-23, wherein the first and/or second capture micro-object has a largest dimension from 1 μm to 50 μm, from 5 μm to 40 μm, from 10 μm to 30 μm, or from 10 μm to 25 μm.

Embodiment 25. The method of any one of embodiments 1-24, wherein the first and/or second capture micro-object comprises a microparticle, a microbead, or a magnetic bead.

Embodiment 26. The method of any one of embodiments 1-25, wherein the first capture micro-object comprises a plurality of first capture micro-objects, each comprising the first molecule.

Embodiment 27. The method of embodiment 26, further comprising allowing the second molecule to bind to the first molecule of each of the plurality of first capture micro-objects, wherein the binding of the second molecule to the first molecule is allowed to proceed to saturation.

Embodiment 28. The method of embodiment 27, further comprising detecting over a period of time a decrease in the amount of second molecule bound to the plurality of first capture micro-objects.

Embodiment 29. The method of embodiment 28, further comprising
determining the relative binding affinity between the first molecule and the second molecule based on a ratio of (i) the increase in the amount of second molecule bound to the second capture micro-object over the period of time to (ii) the decrease in amount of second molecule bound to each of the plurality of first capture micro-objects over the period of time; or
determining the relative binding affinity between the first molecule and the second molecule based on a ratio of (i) the increase in the amount of second molecule bound to the second capture micro-object over the period of time to (ii) the total decrease in the amount of second molecule bound to the plurality of first capture micro-objects over the period of time.

Embodiment 30. The method of any one of the preceding embodiments, wherein the second capture micro-object comprises a plurality of second capture micro-objects, each comprising the first molecule.

Embodiment 31. The method of embodiment 30, further comprising detecting over a period of time an increase in the amount of second molecule bound to the plurality of second capture micro-objects.

Embodiment 32. The method of any one of embodiments 1-31, further comprising calculating the binding affinity between the first molecule and the second molecule based on a ratio of (i) the total increase in the amount of second molecule bound to the plurality of second capture micro-objects over the period of time to (ii) the decrease in the amount of second molecule bound to the first capture micro-object over the period of time.

Embodiment 33. A method for assaying binding affinities of a target molecule and each of a plurality of distinct binding partners in a micro-fluidic device, wherein the micro-fluidic device comprises a flow region and a plurality of chambers that open off of the flow region, the method comprising:
providing the target molecule into the plurality of chambers, wherein the target molecule is labeled with a signal-emitting moiety and wherein a first plurality of capture micro-objects, each comprising a distinct binding partner, are present in the plurality of chambers; and allowing the target molecule to bind to the binding partners of the capture micro-objects of the first plurality, wherein the binding of the target molecule to the binding partners is allowed to proceed to saturation;
removing unbound target molecule from the microfluidic device;
providing a second plurality of capture micro-objects into the plurality of chambers, wherein each of the capture micro-objects of the second plurality comprises a binding partner for the target molecule;
detecting over a period of time a decrease in the amount of target molecule bound to the capture micro-objects of the first plurality;
optionally detecting over the period of time an increase in the amount of target molecule bound to the capture micro-objects of the second plurality;
determining relative binding affinities of the target molecule and each of the plurality of distinct binding partners based on (1) decreases in the amount of target molecule bound to the capture micro-objects of the first plurality over the period of time, or (2) ratios of (i) increases in the amount of target molecule bound to the capture micro-objects of the second plurality over the period of time to (ii) decreases in the amount of target molecule bound to the capture micro-objects of the first plurality over the period of time.

Embodiment 34. The method of embodiment 33, wherein the capture micro-objects of the first plurality comprise distinct binding partners and, optionally, wherein the distinct binding partners are distinctly labeled.

Embodiment 35. The method of embodiment 34, wherein the capture micro-objects of the second plurality comprise distinct binding partners and, optionally, wherein the distinct binding partners are distinctly labeled.

Embodiment 36. The method of embodiment 34, wherein the binding partner is identical, or substantially the same for each capture micro-object of the second plurality.

Embodiment 37. The method of embodiment 34, wherein the binding partner for each corresponding capture micro-object of the second plurality binds to an epitope on the target molecule, wherein the epitope is substantially the same for each binding partner and its corresponding capture micro-object.

Embodiment 38. The method of any one of embodiments 33-37, comprising providing the capture micro-objects of the first plurality into the plurality of chambers prior to providing the target molecule into the plurality of chambers.

Embodiment 39. The method of any one of embodiments 33-38, wherein the plurality of chambers is a first plurality of chambers and wherein prior to providing the first plurality of capture micro-objects into the first plurality of chambers, the method comprises disposing the first plurality of capture micro-objects into a second plurality of chambers in which the distinct binding partners are present, and allowing the binding partners to bind to the capture micro-objects of the first plurality in the second plurality of chambers.

Embodiment 40. The method of any one of embodiments 33-39, further comprising, prior to and/or simultaneously with allowing the binding partners to bind to the capture micro-objects of the first plurality in the second plurality of chambers:
culturing a plurality of biological cells in the second plurality of chambers, wherein the plurality of biological cells secrete the binding partners.

Embodiment 41. The method of any one of embodiments 39 or 40, wherein each chamber of the first plurality is adjacent to a chamber of the second plurality, and providing the first plurality of capture micro-objects into the first plurality of chambers comprises moving the capture micro-objects of the first plurality from a chamber of the second plurality into the adjacent chamber of the first plurality.

Embodiment 42. A method for assaying binding affinities of a target molecule and one or more binding partners for the target molecule in a micro-fluidic device, wherein the micro-fluidic device comprises a flow region and a chamber that opens off of the flow region, the method comprising:
providing the target molecule into the chamber, wherein the target molecule is labeled with a signal-emitting moiety and wherein a first capture micro-object comprising a first binding partner is present in the chamber; and allowing the target molecule to bind to the first binding partner of the first capture micro-object, wherein the binding of the target molecule to the first binding partner is allowed to proceed to saturation;
removing unbound target molecule from the microfluidic device;
providing a second capture micro-object into the chamber, wherein the second capture micro-object comprises a second binding partner different from the first binding partner; detecting over a period of time a decrease in the amount of target molecule bound to the first capture micro-object;
optionally detecting over the period of time an increase in the amount of target molecule bound to the second capture micro-object;
determining a relative binding affinity of the target molecule and the first binding partner based on (1) the decrease in the amount of target molecule bound to the first capture micro-object over the period of time, or (2) a ratio of (i) the increase in the amount of target molecule bound to the second capture micro-object over the period of time to (ii) the decrease in the amount of target molecule bound to the first capture micro-object over the period of time.

Embodiment 43. The method of claim 42, wherein a binding partner with a known k-off is used for the second binding partner of the second capture micro-object.

Embodiment 44. The method of embodiment 42, further comprising calculating k_{off} for the second binding partner of the second capture micro-object based on the ratio of (i) the increase in the amount of target molecule bound to the second capture micro-object over the period of time to (ii) the decrease in the amount of target molecule bound to the first capture micro-object over the period of time.

Embodiment 45. The method of any one of embodiments 42-44, wherein the micro-fluidic device comprises a second chamber that opens off of the flow region, and the method further comprises
providing the target molecule into the second chamber, wherein a third capture micro-object comprising a third binding partner different from the first binding partner is present in the second chamber; and allowing the target molecule to bind to the third binding partner of the third capture micro-object, wherein the binding of the target molecule to the third binding partner is allowed to proceed to saturation;
removing unbound target molecule from the microfluidic device;
providing an additional second capture micro-object into the second chamber, wherein the additional second capture micro-object comprises the second binding partner;
detecting over a period of time a decrease in the amount of target molecule bound to the third capture micro-object;
optionally detecting over the period of time an increase in the amount of target molecule bound to the additional second capture micro-object;
determining a relative binding affinity of the target molecule and the third binding partner based on (1) the decrease in the amount of target molecule bound to the third capture micro-object over the period of time, or (2) a ratio of (i) the increase in the amount of target molecule bound to the additional second capture micro-object over the period of time to (ii) the decrease in the amount of target molecule bound to the third capture micro-object over the period of time.

Embodiment 46. The method of any one of embodiments 1-45, wherein the microfluidic device comprises a housing, wherein the housing comprises a base and a microfluidic structure disposed on the base.

Embodiment 47. The method of any one of embodiments 1-46 wherein the flow path comprises a microfluidic channel, and wherein the chamber opens off of the microfluidic channel.

Embodiment 48. The method of 1-47, wherein the chamber is micro-well formed in the base of the housing.

Embodiment 49. The method of any one of embodiments 1-48, wherein the chamber is a sequestration pen.

Embodiment 50. The method of embodiment 49, wherein each sequestration pen comprises an isolation region having a single opening, and a connection region, the connection region having a proximal opening to the flow region (or channel) and a distal opening to the isolation region, optionally wherein the isolation region is an unswept region of the microfluidic device.

Embodiment 51. The method of embodiment 50, wherein the connection region comprises a proximal opening into the flow region (or microfluidic channel) having a width W_{con} ranging from about 20 microns to about 100 microns and a distal opening into said isolation region, and wherein a length L_{con} of said connection region from the proximal opening to the distal opening is as least 1.0 times a width con of W_{con} the proximal opening of the connection region.

Embodiment 52. The method of embodiment 51, wherein the length L_{con} of the connection region from the proximal opening to the distal opening is at least 1.5 times the width W_{con} of the proximal opening of the connection region.

Embodiment 53. The method of embodiment 52, wherein the length L_{con} of the connection region from the proximal opening to the distal opening is at least 2.0 times the width W_{con} of the proximal opening of the connection region.

Embodiment 54. The method of any one of embodiments 51-53, wherein the width W_{con} of the proximal opening of the connection region ranges from about 20 microns to about 60 microns.

Embodiment 55. The method of any one of embodiments 51-54, wherein the length L_{con} of the connection region from the proximal opening to the distal opening is between about 20 microns and about 500 microns.

Embodiment 56. The method of any one of embodiments 50-55, wherein a width of the microfluidic channel at the proximal opening of the connection region is between about 50 microns and about 500 microns.

Embodiment 57. The method of any one of embodiments 50-56, wherein a height of the microfluidic channel at the proximal opening of the connection region is between 20 microns and 100 microns.

Embodiment 58. The method of any one of embodiments 50-57, wherein the proximal opening of the connection region is parallel to a direction of the flow of a first medium in the flow region.

Embodiment 59. The method of any one of embodiments 50-58, wherein the width of the isolation region at the distal opening is substantially the same as the width of the connection region at the proximal opening, and larger than the largest dimension of the first and second capture micro-objects.

Embodiment 60. The method of any one of embodiments 50-59, wherein during the detecting step, the first capture micro-object and the second capture micro-object are present in the isolation region of the chamber.

Embodiment 61. The method of any one of embodiments 50-60, wherein the distance between the first capture micro-object and the second capture micro-object (D_{L}) is equal to or smaller than the entire length of the isolation region.

Embodiment 62. The method of embodiment 61, wherein the distance of the second capture micro-object from the proximal opening of the connection region (D_{d}) is smaller than the distance of the first capture micro-object from the proximal opening of the connection region (D_{d}+D_{L}).

Embodiment 63. The method of any one of embodiments 61 or 62, wherein the D_{L} is about 20 microns to about 200 microns, 20 microns to 180 microns, 20 microns to 160 microns, 20 microns to 140 microns, 20 microns to 120 microns, 20 microns to 100 microns, 20 microns to 90 microns, 30 microns to 200 microns, 30 microns to 180 microns, 30 microns to 160 microns, 30 microns to 140 microns, 30 microns to 120 microns, 30 microns to 100 microns, 30 microns to 90 microns, 40 microns to 200 microns, 40 microns to 180 microns, 40 microns to 160 microns, 40 microns to 140 microns, 40 microns to 120 microns, 40 microns to 100 microns, 40 microns to 90 microns, 40 microns to 60 microns, 50 microns to 200 microns, 50 microns to 180 microns, 50 microns to 160 microns, 50 microns to 140 microns, 50 microns to 120 microns, 50 microns to 100 microns, 50 microns to 90 microns, 60 microns to 200 microns, 60 microns to 180 microns, 60 microns to 160 microns, 60 microns to 140 microns, 60 microns to 120 microns, 60 microns to 100 microns, 60 microns to 90 microns, 80 microns to 200 microns, 80 microns to 180 microns, 80 microns to 160 microns, 80 microns to 140 microns, 80 microns to 120 microns, 80 microns to 100 microns, 80 microns to 90 microns, or about 90 microns.

Embodiment 64. The method of any one of embodiments 50-63, wherein the second capture micro-object is separated from the connection region by a distance, D_{c}, whereas D_{c} is equal to or larger than 10 microns (e.g., at least 15 microns, 20 microns, 25 microns, 30 microns, 35 microns, 40 microns, 45 microns, 50 microns, or more).

Embodiment 65. The method of any one of embodiments 50-64, wherein the distance of the second capture micro-object from the proximal opening of the connection region (D_{d}) is longer than the penetration depth (Dₚ) of the first fluidic medium flowing from the flowing region.

Embodiment 66. The method of any one of embodiments 50-65, wherein the isolation region of the sequestration pen has a length of about 40-600 microns, about 40-500 microns, about 40-400 microns, about 40-300 microns, about 40-200 microns, about 40-100 microns, about 40-80 microns, about 30-550 microns, about 30-450 microns, about 30-350 microns, about 30-250 microns, about 30-170 microns, about 30-80 microns or about 30-70 microns.

Embodiment 67. The method of any one of embodiments 50-66, wherein the isolation region of the sequestration pen has a length of about 40-100 microns, about 40-80 microns, about 30-80 microns or about 30-70 microns.

Embodiment 68. The method of any one of embodiments 50-67, wherein D_{L} is in a range from a first fraction to a second fraction of the length of the isolation region, wherein the first and second fraction are respectively 0.1 and 0.2; 0.2 and 0.3; 0.3 and 0.4; 0.4 and 0.5; 0.5 and 0.6; 0.6 and 0.7; 0.7 and 0.8; 0.8 and 0.9; or 0.9 and 1.

Embodiment 69. The method of any one of embodiments 50-68, wherein during the detecting step, the first capture micro-object and the plurality of second capture micro-objects are present in the isolation region of the chamber.

Embodiment 70. The method of embodiment 69, wherein the plurality of second capture micro-objects are proximal to the proximal opening of the connection region and the first capture micro-object is distal from the proximal opening of the connection region.

Embodiment 71. The method of embodiment 70, wherein the plurality of second capture micro-objects include a most proximal second capture micro-object and a most distal second capture micro-object, defining a distance therebetween, H_{c}.

Embodiment 72. The method of embodiment 71, wherein the sum of the distance H_{c} and the distance between the most proximal first capture micro-object and the first capture micro-object is smaller than the entire length of the isolation region.

Embodiment 73. The method of any one of embodiments 50-68, wherein during the detecting step, the plurality of first capture micro-objects and the second capture micro-object are present in the isolation region of the chamber.

Embodiment 74. The method of embodiment 73, wherein the second capture micro-object from the proximal opening of the connection region is proximal to the proximal opening of the connection region and the plurality of first capture micro-objects are distal from the proximal opening of the connection region.

Embodiment 75. The method of embodiment 74, wherein the plurality of first capture micro-objects include a most proximal first capture micro-object and a most distal first capture micro-object, defining a distance therebetween, H_{c}.

Embodiment 76. The method of embodiment 75, wherein the sum of the distance H_{c} and the distance between the most proximal capture micro-object and the second capture micro-object is smaller than the entire length of the isolation region.

Embodiment 77. The method of any one of embodiments 50-76, wherein the proximal opening of the connection region is parallel to the direction of the flow of the first medium, and the distal opening of the isolation region is not parallel to the direction of the flow of the first medium.

Embodiment 78. The method of embodiment 77, wherein the width W_{con2} of the distal opening of the connection region is substantially the same as the width W_{con1} of the proximal opening of the connection region, and is larger than the largest dimension of the first and second capture micro-objects.

Embodiment 79. The method of embodiment 77, wherein the width W_{con2} of the distal opening of the connection region is larger or smaller as the width W_{con1} of the proximal opening of the connection region, and is larger than the largest dimension of the first and second capture micro-objects.

Embodiment 80. The method of any one of embodiments 50-68, wherein during the detecting step, the first capture micro-object and the second capture micro-object are present in the isolation region of the sequestration pen.

Embodiment 81. The method of embodiment 80, wherein the distance between the first capture micro-object and the second capture micro-object in a direction parallel to the length of the connection region, D_{L}, is equal to or smaller than the entire length of the isolation region.

Embodiment 82. The method of embodiment 81, wherein the distance between the first capture micro-object and the second capture micro-object in a direction parallel to the width of the proximal opening of the connection region, D_{L}, is equal to or smaller than the width between opposite walls of the isolation region.

Embodiment 83. The method of any one of embodiments 49-82, wherein the sequestration pen comprises a connection region wall laterally positioned with respect to the proximal opening and at least partially extends into the enclosed portion of the sequestration pen with the length L_{wall}, defining a hook region in the isolation region.

Embodiment 84. The method of embodiment 83, wherein the first capture micro-object is present in or proximal to the hook region, and the second capture micro-object is distal from the hook region.

Embodiment 85. The method of any one of embodiments 1-84, wherein the inner surface of the chamber or sequestration pen is treated with a coating material for linking the first and/or second capture micro-object to the inner surface prior to introducing the first and/or second capture micro-object into the chamber.

Embodiment 86. The method of embodiment 85, wherein the first and/or second capture micro-object is covalently linked to the inner surface treated with the coating material.

Embodiment 87. The method of embodiment 85, wherein the first and/or second capture micro-object is non-covalently linked to the inner surface treated with the coating material.

### EXAMPLES

The following examples are provided to illustrate certain embodiments of the disclosure and do not limit the disclosure or the scope of the claims.

### Example 1

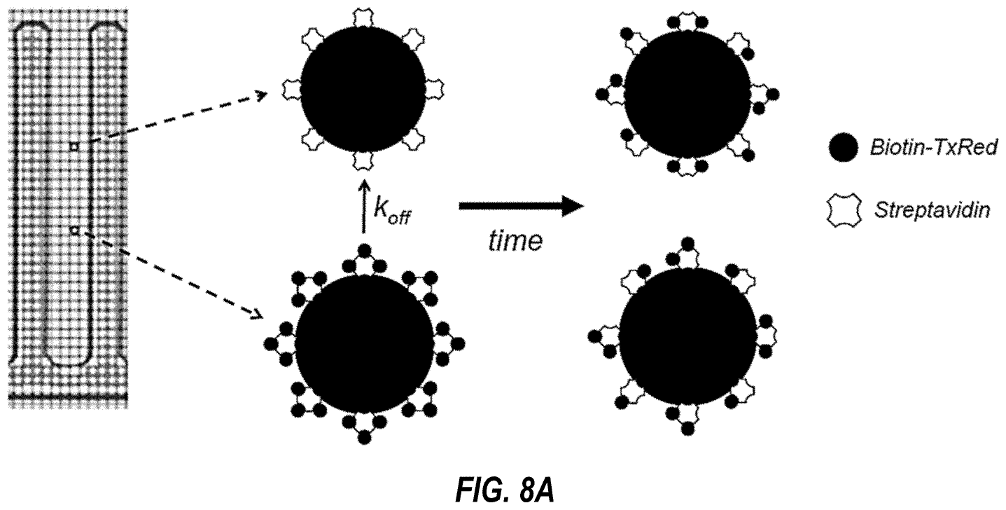

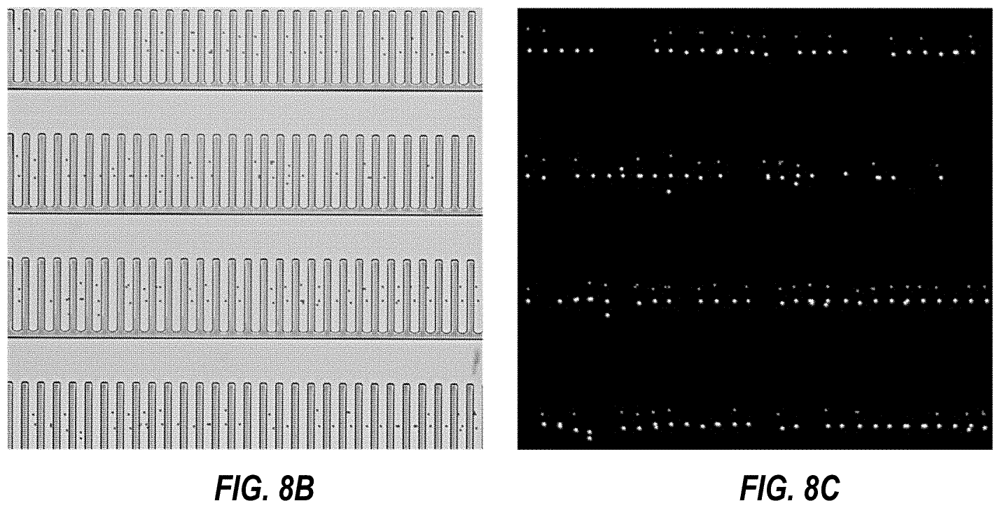

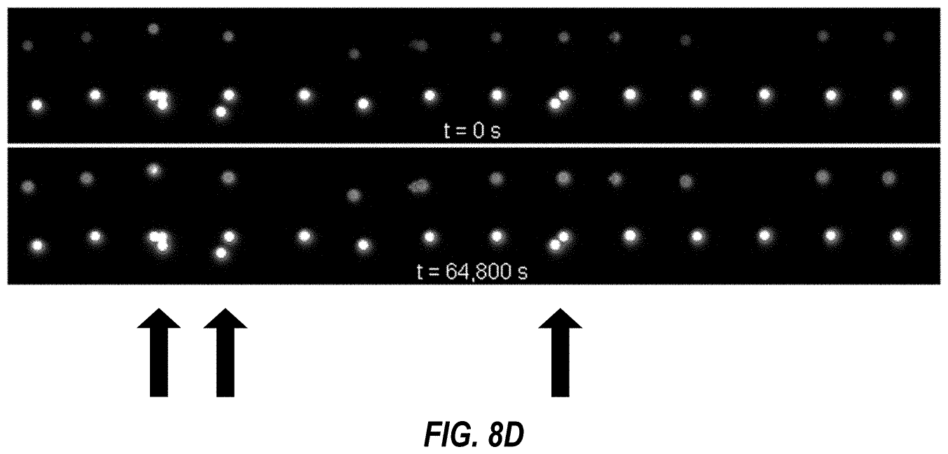

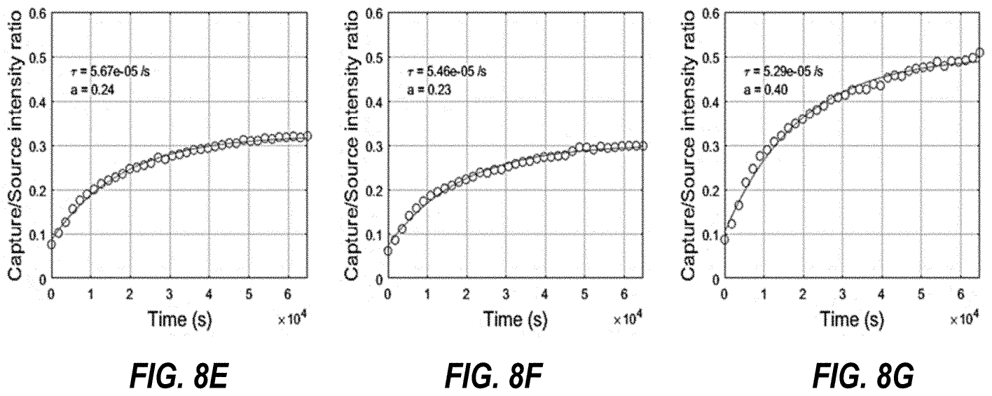

An example of the method described herein was performed in a micro-fluidic device (OptoSelect™ chip, Berkeley Lights, Inc.) for assaying a binding affinity between a first molecule and a second molecule. To verify the method, binding of biotin and streptavidin was employed, since their binding interactions are well characterized. As shown in FIG. 8A for an exemplary chamber (or sequestration pen) of the microfluidic device, source beads (first capture micro-objects) coated with streptavidin were contacted with biotin labeled with a fluorophore (Texas Red) in the chambers. The biotin-streptavidin binding proceeded to saturation. Unbound biotin was removed from the microfluidic device. Capture beads (second capture micro-objects) coated with streptavidin only (without biotin) were provided into the chambers where the source beads were present (FIG. 8B).

Fluorescent imaging was performed to detect fluorescent intensities from the source bead and the capture bead in each chamber (FIG. 8C). Fluorescent intensity was measured periodically over an 18-hour period of time to detect the changes in the source bead and in the capture bead, respectively in each sequestration pen (FIG. 8D). An exponential fit function with an offset (y=a(1−exp(−t/τ))+b), was used to fit the calculated ratios of fluorescent intensity of the capture bead to the source bead over time (t). The ratio of fluorescent intensity of the capture bead to the source bead increased with a rising exponential whose rate constant (τ) reflects k_{off} (FIG. 8E-8G). Based on these data, the k_{off} values were calculated as 5.29×10⁻⁵ s⁻¹-5.67 10⁻⁵ s⁻¹, as compared to a reported k_{off} value of 5.0×10⁻⁵ s⁻¹ (Deng et al., J. Am. Soc. Mass Spectrom. (2013) 24:49-56).

### Example 2

An example of the method descried herein was performed for assaying binding affinities of a target molecule and each of a plurality of distinct binding partners in a microfluidic device (an OptoSelect™ chip, Berkeley Lights, Inc.) having a flow region and a plurality of chambers (or sequestration pens) that open off of the flow region. A first plurality of capture micro-objects (source beads), each coated with the same binding partner, were present in the plurality of chambers. The target molecules were labeled with a fluorophore (Texas Red) and provided into the plurality of chambers, allowing the target molecules to bind to the binding partners of the source beads. The binding of the target molecules to the binding partners proceeded to saturation. Unbound target molecule was removed from the microfluidic device. Then, a second plurality of capture micro-objects (capture beads) were provided into the plurality of chambers. Each of the capture beads was coated with the same binding partner (which was also the same as the binding partner of the source beads). The changes in the fluorescent intensity in each of the source beads and capture beads were measured over time.

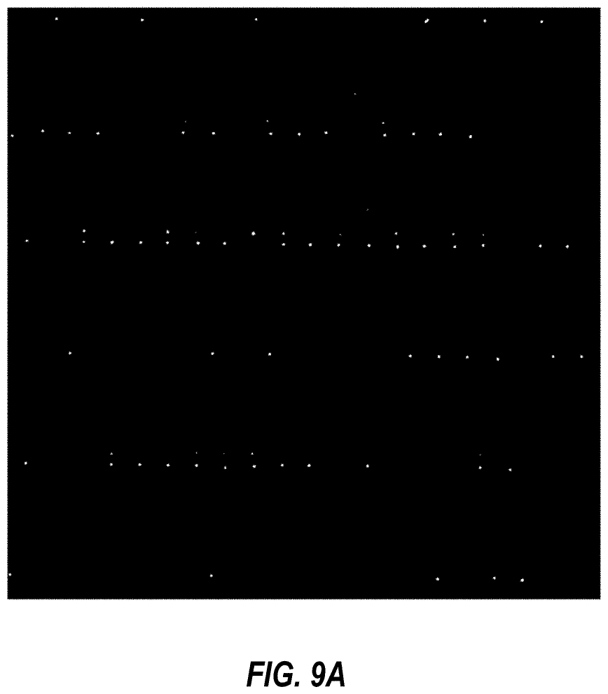

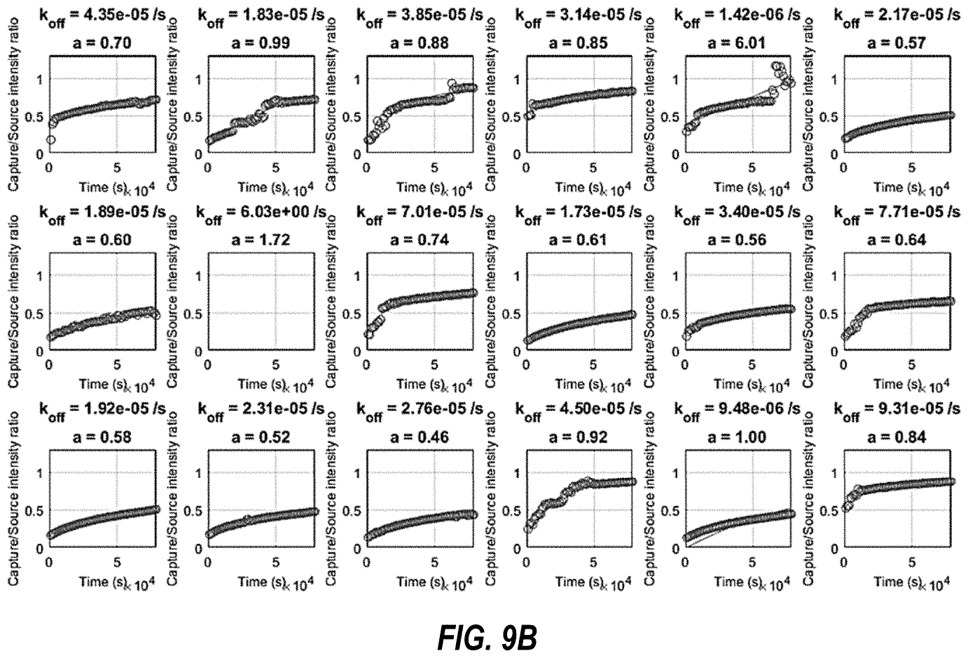

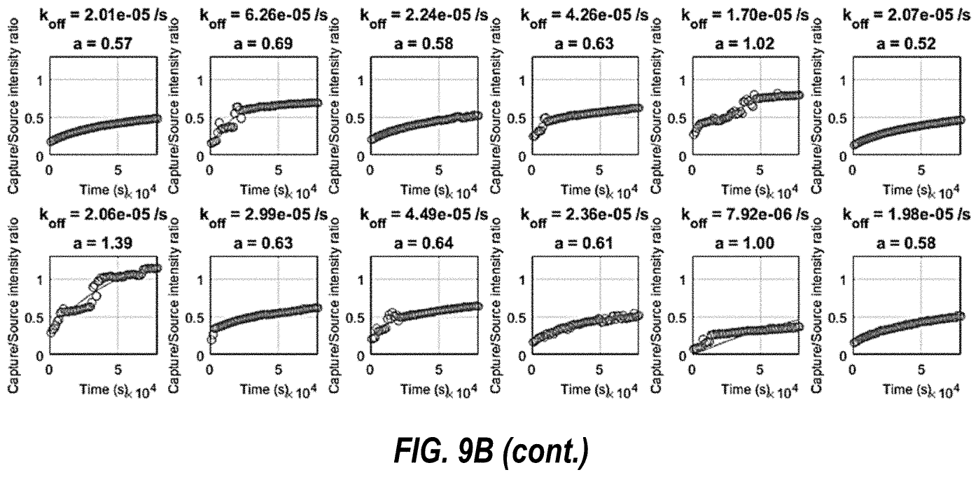

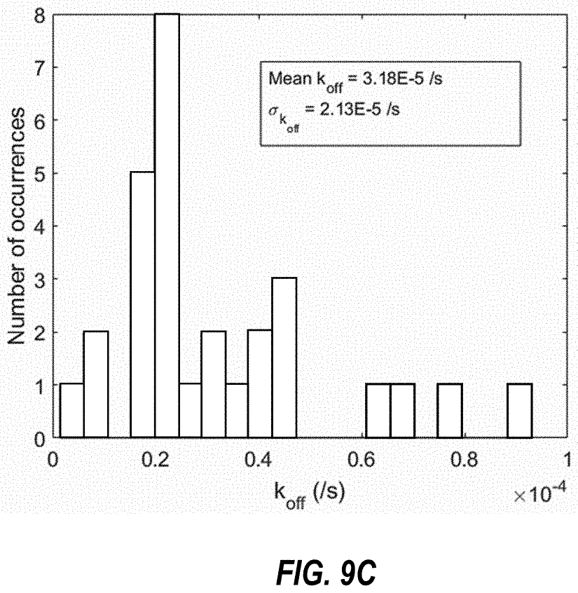

FIGS. 9A-9C show an array of capture/source beads coated with binding partners in which independent measurements of k_{off} for the interaction of the same target molecule to the binding partners were made in parallel. The ratio of the capture bead/source bead fluorescent intensity was plotted over time. As shown in FIG. 9B, in the same chamber, the ratio of the capture bead/source bead fluorescent intensity increased over time, confirming that the occupancy of the target molecule on the capture bead increased over time while the occupancy of the target molecule on the source bead decreased over time. An exponential fit function with an offset (y=a(1−exp(−t/τ))+b), was used to fit the calculated ratios of fluorescent intensity of the capture bead to the source bead over time (t). The capture/source intensity ratio increased with an exponential whose rate constant (τ) reflects k_{off} (FIG. 9B). In each of the plots shown in FIG. 9B, the x-axis indicates time (×10⁴ s) and the y-axis indicates the capture/source intensity ratio. FIG. 9C shows a histogram of measured k_{off} for the pairs of the source/capture beads, as well as a calculation of the standard deviation (sigma) for the set of k_{off} measurements.

Variations to the foregoing example are possible, and include, for example, using source beads coated with distinct binding partners. This allows for many different binding interactions to be assayed simultaneously. In particular, the source beads can be coated with different antibodies (e.g., each antibody differing with regard to 1, 2, 3, 4, 5, 6, 7, 8, 9, 10, or more amino acid residues in its variable region, as might occur during an antibody engineering campaign). Alternatively, or in addition, the capture beads can be coated with distinct binding partners.

1. A method for assaying a binding affinity between a first molecule and a second molecule in a micro-fluidic device, wherein the micro-fluidic device comprises a flow region and a chamber that opens off of the flow region, the method comprising:
providing the second molecule into the chamber, wherein the second molecule is labeled with a signal-emitting moiety and a first capture micro-object comprising the first molecule is present in the chamber, and allowing the second molecule to bind to the first molecule of the first capture micro-object, wherein the binding of the second molecule to the first molecule is allowed to proceed to saturation;
removing unbound second molecule from the microfluidic device;
providing a second capture micro-object into the chamber, wherein the second capture micro-object comprises a third molecule which specifically binds to the second molecule;
detecting over a period of time a decrease in an amount of second molecule bound to the first capture micro-object;
optionally detecting over the period of time an increase in the amount of second molecule bound to the second capture micro-object; and
determining a relative binding affinity between the first molecule and the second molecule based on one of the following:
i. the decrease in the amount of second molecule bound to the first capture micro-object over the period of time; or
ii. a ratio of (i) the increase in the amount of second molecule bound to the second capture micro-object over the period of time to (ii) the decrease in the amount of second molecule bound to the first capture micro-object over the period of time.

2. A method for assaying a binding affinity between a first molecule and a second molecule in a micro-fluidic device, wherein the micro-fluidic device comprises a flow region, a chamber that opens off of the flow region, the method comprising:
providing a second molecule labeled with a signal-emitting moiety into the chamber, wherein a first capture micro-object comprising the first molecule is present in the chamber, and allowing the second molecule to bind to the first molecule of the first capture micro-object, wherein the binding of the second molecule to the first molecule is allowed to proceed to saturation;
removing unbound second molecule from the microfluidic device;
detecting over a period of time a decrease in the amount of the second molecule bound to the first capture micro-object; and
determining a relative binding affinity between the first molecule and the second molecule based on the decrease in the amount of the second molecule bound to the first capture micro-object over the period of time.

3. The method of claim 1, wherein determining the relative binding affinity between the first molecule and the second molecule comprises calculating a dissociation rate constant (k_{off}) for the first and second molecules or dividing the dissociation rate constant (k_{off}) for the first and second molecules by an association rate constant (kₒₙ).

4. (canceled)

5. The method of claim 3, wherein kₒₙ is an estimated value based on known association rate constants for molecules similar to the first and second molecules or a kₒₙ value is in the range of about 1×10⁶ to about 1×10⁷ M⁻¹ s⁻¹.

6. (canceled)

7. (canceled)

8. The method of claim 1, further comprising, prior to providing the second molecule into the chamber, providing the first capture micro-object into the chamber.

9. The method of claim 8, wherein the chamber is a first chamber and wherein prior to providing the first capture micro-object into the chamber, the method comprises disposing a capture micro-object into a second chamber in which the first molecule is present, and allowing the first molecule to bind to the capture micro-object in the second chamber and thereby generate the first capture micro-object, optionally wherein the second chamber is adjacent to the first chamber.

10. The method of claim 9, further comprising, prior to and/or simultaneously with allowing the first molecule to bind to the capture micro-object in the second chamber:
culturing one or more biological cells in the second chamber, wherein the one or more biological cells secrete the first molecule.

11. The method of claim 1, wherein the first molecule is an antibody or an antigen-binding fragment thereof.

12. The method of claim 11, wherein the third molecule is substantially identical to the first molecule.

13. The method of claim 1, wherein the first capture micro-object comprises a plurality of first capture micro-objects, each comprising the first molecule.

14. The method of claim 13, further comprising allowing the second molecule to bind to the first molecule of each of the plurality of first capture micro-objects, wherein the binding of the second molecule to the first molecule is allowed to proceed to saturation; and
comprising detecting over a period of time a decrease in the amount of second molecule bound to the plurality of first capture micro-objects.

15. (canceled)

16. The method of claim 14, further comprising
determining the relative binding affinity between the first molecule and the second molecule based on a ratio of (i) the increase in the amount of second molecule bound to the second capture micro-object over the period of time to (ii) the decrease in amount of second molecule bound to each of the plurality of first capture micro-objects over the period of time; or
determining the relative binding affinity between the first molecule and the second molecule based on a ratio of (i) the increase in the amount of second molecule bound to the second capture micro-object over the period of time to (ii) the total decrease in the amount of second molecule bound to the plurality of first capture micro-objects over the period of time.

17. The method of claim 1, wherein the second capture micro-object comprises a plurality of second capture micro-objects, each comprising the first molecule.

18. The method of claim 1, further comprising calculating the binding affinity between the first molecule and the second molecule based on a ratio of (i) the total increase in the amount of second molecule bound to the plurality of second capture micro-objects over the period of time to (ii) the decrease in the amount of second molecule bound to the first capture micro-object over the period of time.

19. A method for assaying binding affinities of a target molecule and each of a plurality of distinct binding partners in a micro-fluidic device, wherein the micro-fluidic device comprises a flow region and a plurality of chambers that open off of the flow region, the method comprising:
providing the target molecule into the plurality of chambers, wherein the target molecule is labeled with a signal-emitting moiety and wherein a first plurality of capture micro-objects, each comprising a distinct binding partner, are present in the plurality of chambers; and allowing the target molecule to bind to the binding partners of the capture micro-objects of the first plurality, wherein the binding of the target molecule to the binding partners is allowed to proceed to saturation;
removing unbound target molecule from the microfluidic device;
providing a second plurality of capture micro-objects into the plurality of chambers, wherein each of the capture micro-objects of the second plurality comprises a binding partner for the target molecule;
detecting over a period of time a decrease in the amount of target molecule bound to the capture micro-objects of the first plurality;
optionally detecting over the period of time an increase in the amount of target molecule bound to the capture micro-objects of the second plurality;
determining relative binding affinities of the target molecule and each of the plurality of distinct binding partners based on (1) decreases in the amount of target molecule bound to the capture micro-objects of the first plurality over the period of time, or (2) ratios of (i) increases in the amount of target molecule bound to the capture micro-objects of the second plurality over the period of time to (ii) decreases in the amount of target molecule bound to the capture micro-objects of the first plurality over the period of time.

20.-24. (canceled)

25. A method for assaying binding affinities of a target molecule and one or more binding partners for the target molecule in a micro-fluidic device, wherein the micro-fluidic device comprises a flow region and a chamber that opens off of the flow region, the method comprising:
providing the target molecule into the chamber, wherein the target molecule is labeled with a signal-emitting moiety and wherein a first capture micro-object comprising a first binding partner is present in the chamber; and allowing the target molecule to bind to the first binding partner of the first capture micro-object, wherein the binding of the target molecule to the first binding partner is allowed to proceed to saturation;
removing unbound target molecule from the microfluidic device;
providing a second capture micro-object into the chamber, wherein the second capture micro-object comprises a second binding partner different from the first binding partner;
detecting over a period of time a decrease in the amount of target molecule bound to the first capture micro-object;
optionally detecting over the period of time an increase in the amount of target molecule bound to the second capture micro-object;
determining a relative binding affinity of the target molecule and the first binding partner based on (1) the decrease in the amount of target molecule bound to the first capture micro-object over the period of time, or (2) a ratio of (i) the increase in the amount of target molecule bound to the second capture micro-object over the period of time to (ii) the decrease in the amount of target molecule bound to the first capture micro-object over the period of time.

26. (canceled)

27. The method of claim 25, wherein the micro-fluidic device comprises a second chamber that opens off of the flow region, and the method further comprises
providing the target molecule into the second chamber, wherein a third capture micro-object comprising a third binding partner different from the first binding partner is present in the second chamber; and allowing the target molecule to bind to the third binding partner of the third capture micro-object, wherein the binding of the target molecule to the third binding partner is allowed to proceed to saturation;
removing unbound target molecule from the microfluidic device;
providing an additional second capture micro-object into the second chamber, wherein the additional second capture micro-object comprises the second binding partner;
detecting over a period of time a decrease in the amount of target molecule bound to the third capture micro-object;
optionally detecting over the period of time an increase in the amount of target molecule bound to the additional second capture micro-object;
determining a relative binding affinity of the target molecule and the third binding partner based on (1) the decrease in the amount of target molecule bound to the third capture micro-object over the period of time, or (2) a ratio of (i) the increase in the amount of target molecule bound to the additional second capture micro-object over the period of time to (ii) the decrease in the amount of target molecule bound to the third capture micro-object over the period of time.

28. The method of claim 1, wherein the chamber is a sequestration pen,
wherein each sequestration pen comprises an isolation region having a single opening, and a connection region, the connection region having a proximal opening to the flow region and a distal opening to the isolation region, optionally wherein the isolation region is an unswept region of the microfluidic device.

29. (canceled)

30. The method of claim 28, wherein the connection region comprises a proximal opening into the flow region (or microfluidic channel) having a width W_{con} ranging from about 20 microns to about 100 microns and a distal opening into said isolation region, and wherein a length L_{con} of said connection region from the proximal opening to the distal opening is as least 1.0 times a width W_{con} of the proximal opening of the connection region.

31. (canceled)

32. (canceled)

33. The method of claim 28, wherein the width of the isolation region at the distal opening is substantially the same as the width of the connection region at the proximal opening, and larger than the largest dimension of the first and second capture micro-objects.

34. The method of claim 28, wherein during the detecting step, the first capture micro-object and the second capture micro-object are present in the isolation region of the chamber.

35. The method of claim 28, wherein the distance between the first capture micro-object and the second capture micro-object (D_{L}) is equal to or smaller than the entire length of the isolation region, and the distance of the second capture micro-object from the proximal opening of the connection region (D_{d}) is smaller than the distance of the first capture micro-object from the proximal opening of the connection region (D_{d}+D_{L}).

36.-42. (canceled)
